(19) United States
(12) Patent Application Publication
(10) Pub. No.: US 2026/0198930 A1
(43) Pub. Date: Jul. 16, 2026

(54) Steerable and/or Radially Asymmetric Intrasaccular Aneurysm Occlusion Devices

(71) Applicant: Robert A. Connor, Wyoming, MN (US)

(72) Inventor: Robert A. Connor, Wyoming, MN (US)

(73) Assignee: Aneuclose LLC, Ham Lake, MN (US)

(21) Appl. No.: 19/551,499

(22) Filed: Feb. 26, 2026

Related U.S. Application Data
(63) Continuation-in-part of application No. 19/274,599, filed on Jul. 20, 2025.
(63) Continuation-in-part of application No. 19/023,514, filed on Jan. 16, 2025.
(63) Continuation-in-part of application No. 18/920,939, filed on Oct. 20, 2024.
Application No. 19/274,599 is a continuation-in-part of application No. 19/023,514, filed on Jan. 16, 2025.
Application No. 19/023,514 is a continuation-in-part of application No. 18/920,939, filed on Oct. 20, 2024.
Application No. 19/023,514 is a continuation-in-part of application No. 18/920,939, filed on Oct. 20, 2024.
Application No. 18/920,939 is a continuation-in-part of application No. 18/760,322, filed on Jul. 1, 2024, now abandoned.
Application No. 18/920,939 is a continuation-in-part of application No. 18/760,322, filed on Jul. 1, 2024, now abandoned.
Application No. 18/760,322 is a continuation-in-part of application No. 18/674,996, filed on May 27, 2024, now abandoned.
Application No. 18/760,322 is a continuation-in-part of application No. 18/674,996, filed on May 27, 2024, now abandoned.
Application No. 18/674,996 is a continuation-in-part of application No. 18/613,053, filed on Mar. 21, 2024, now abandoned.
Application No. 18/613,053 is a continuation-in-part of application No. 17/970,510, filed on Oct. 20, 2022, now abandoned.
Application No. 18/674,996 is a continuation-in-part of application No. 18/519,055, filed on Nov. 26, 2023, now Pat. No. 12,004,750.
Application No. 18/613,053 is a continuation-in-part of application No. 18/519,055, filed on Nov. 26, 2023, now Pat. No. 12,004,750.
Application No. 18/519,055 is a continuation-in-part of application No. 18/135,153, filed on Apr. 15, 2023, now abandoned.
Application No. 18/519,055 is a continuation-in-part of application No. 18/374,602, filed on Sep. 28, 2023, now Pat. No. 11,944,316.
Application No. 18/374,602 is a continuation-in-part of application No. 18/135,153, filed on Apr. 15, 2023, now abandoned.
Application No. 18/135,153 is a continuation-in-part of application No. 17/970,510, filed on Oct. 20, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 18/374,602 is a continuation-in-part of application No. 18/135,153, filed on Apr. 15, 2023, now abandoned.
Application No. 18/135,153 is a continuation-in-part of application No. 17/970,510, filed on Oct. 20, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 18/135,153 is a continuation-in-part of application No. 17/970,510, filed on Oct. 20, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 17/829,313 is a continuation-in-part of application No. 17/476,845, filed on Sep. 16, 2021, now Pat. No. 11,583,289.
Application No. 17/965,502 is a continuation-in-part of application No. 17/829,313, filed on May 31, 2022, now abandoned.
Application No. 17/829,313 is a continuation-in-part of application No. 17/476,845, filed on Sep. 16, 2021, now Pat. No. 11,583,289.
Application No. 17/829,313 is a continuation-in-part of application No. 17/485,390, filed on Sep. 25, 2021, now Pat. No. 11,471,164.
Application No. 17/485,390 is a continuation-in-part of application No. 17/476,845, filed on Sep. 16, 2021, now Pat. No. 11,583,289.
Application No. 17/476,845 is a continuation-in-part of application No. 17/472,674, filed on Sep. 12, 2021, now abandoned.
Application No. 17/472,674 is a continuation-in-part of application No. 17/467,680, filed on Sep. 7, 2021, now abandoned.
Application No. 17/467,680 is a continuation-in-part of application No. 17/466,497, filed on Sep. 3, 2021, now Pat. No. 11,357,511.
Application No. 17/466,497 is a continuation-in-part of application No. 17/353,652, filed on Jun. 21, 2021, now abandoned.
Application No. 17/353,652 is a continuation-in-part of application No. 17/220,002, filed on Apr. 1, 2021, now Pat. No. 11,464,518.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/211,446 is a continuation-in-part of application No. 16/693,267, filed on Nov. 23, 2019, now abandoned.
Application No. 16/693,267 is a continuation-in-part of application No. 16/660,929, filed on Oct. 23, 2019, now Pat. No. 11,471,163.
Application No. 16/693,267 is a continuation-in-part of application No. 16/660,929, filed on Oct. 23, 2019, now Pat. No. 11,471,163.
Application No. 16/660,929 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 15/865,822, filed on Jan. 9, 2018, now Pat. No. 10,716,573.
(60) Provisional application No. 62/444,860, filed on Jan. 11, 2017.
(60) Provisional application No. 62/472,519, filed on Mar. 16, 2017.
(60) Provisional application No. 62/472,519, filed on Mar. 16, 2017.
(60) Provisional application No. 62/589,754, filed on Nov. 22, 2017.
(60) Provisional application No. 62/720,173, filed on Aug. 21, 2018.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 63/119,774, filed on Dec. 1, 2020.

(51) Int. Cl. A61B 17/12

(52) U.S. Cl. CPC A61B 17/12113; A61B 17/1214; A61B 2017/1205

(57) ABSTRACT

This invention is an intrasaccular aneurysm occlusion device with an expandable mesh which is inserted and expanded within an aneurysm sac. The expandable mesh can be steered in a selected direction and/or expanded in a radially-asymmetric manner by moving a joint, sliding catheter sections, pulling or pushing wires, detaching constraints, or filling chambers in the mesh with embolic material. There can be a plurality of non-central openings with remotely-controllable valves in the expandable mesh through which embolic material is inserted into the aneurysm sac.

### CROSS-REFERENCE TO RELATED APPLICATIONS

This application is a continuation-in-part of U.S. patent application Ser. No. 19,274,599 filed on 2025 Jul. 20, a continuation-in-part of U.S. patent application Ser. No. 19,023,514 filed on 2025 Jan. 16, and a continuation-in-part of U.S. patent application Ser. No. 18,920,939 filed on 2024 Oct. 20. U.S. patent application Ser. No. 19,274,599 was a continuation-in-part of U.S. patent application Ser. No. 19,0235,14 filed on 2025 Jan. 16 and a continuation-in-part of U.S. patent application Ser. No. 18,920,939 filed on 2024 Oct. 20. U.S. patent application Ser. No. 19,023,514 was a continuation-in-part of U.S. patent application Ser. No. 18,920,939 filed on 2024 Oct. 20 and a continuation-in-part of U.S. patent application Ser. No. 18,760,322 filed on 2024 Jul. 1.

U.S. patent application Ser. No. 18,920,939 was a continuation-in-part of U.S. patent application Ser. No. 18,760,322 filed on 2024 Jul. 1 and a continuation-in-part of U.S. patent application Ser. No. 18,674,996 filed on 2024 May 27. U.S. patent application Ser. No. 18,760,322 was a continuation-in-part of U.S. patent application Ser. No. 18,674,996 filed on 2024 May 27, a continuation-in-part of U.S. patent application Ser. No. 18,613,053 filed on 2024 Mar. 21, and a continuation-in-part of U.S. patent application Ser. No. 17,970,510 filed on 2022 Oct. 20.

U.S. patent application Ser. No. 18,674,996 was a continuation-in-part of Ser. No. 18,613,053 filed on 2024 Mar. 21 and a continuation-in-part of U.S. patent application Ser. No. 18,519,055 filed on 2023 Nov. 26. U.S. patent application Ser. No. 18,613,053 was a continuation-in-part of U.S. patent application Ser. No. 18,519,055 filed on 2023 Nov. 26 and a continuation-in-part of U.S. patent application Ser. No. 18,135,153 filed on 2023 Apr. 15.

U.S. patent application Ser. No. 18,519,055 was a continuation-in-part of U.S. patent application Ser. No. 18,374,602 filed on 2023 Sep. 28, a continuation-in-part of U.S. patent application Ser. No. 18,135,153 filed on 2023 Apr. 15, a continuation-in-part of U.S. patent application Ser. No. 17,970,510 filed on 2022 Oct. 20, a continuation-in-part of U.S. patent application Ser. No. 17,965,502 filed on 2022 Oct. 13, and a continuation-in-part of U.S. patent application Ser. No. 17,829,313 filed on 2022 May 31.

U.S. patent application Ser. No. 18,374,602 was a continuation-in-part of U.S. patent application Ser. No. 18,135,153 filed on 2023 Apr. 15, a continuation-in-part of U.S. patent application Ser. No. 17,970,510 filed on 2022 Oct. 20, a continuation-in-part of U.S. patent application Ser. No. 17,965,502 filed on 2022 Oct. 13, and a continuation-in-part of U.S. patent application Ser. No. 17,829,313 filed on 2022 May 31.

U.S. patent application Ser. No. 18,135,153 was a continuation-in-part of U.S. patent application 17,970,510 filed on 2022 Oct. 20, a continuation-in-part of U.S. patent application Ser. No. 17,965,502 filed on 2022 Oct. 13, and a continuation-in-part of U.S. patent application Ser. No. 17,829,313 filed on 2022 May 31. U.S. patent application Ser. No. 17,970,510 was a continuation-in-part of U.S. patent application Ser. No. 17,965,502 filed on 2022 Oct. 13, a continuation-in-part of U.S. patent application Ser. No. 17,829,313 filed on 2022 May 31, and a continuation-in-part of U.S. patent application Ser. No. 17,476,845 filed on 2021 Sep. 16.

U.S. patent application Ser. No. 17,829,313 was a continuation-in-part of U.S. patent application Ser. No. 17,485,390 filed on 2021 Sep. 25, was a continuation-in-part of U.S. patent application Ser. No. 17,476,845 filed on 2021 Sep. 16, was a continuation-in-part of U.S. patent application Ser. No. 17,472,674 filed on 2021 Sep. 12, was a continuation-in-part of U.S. patent application Ser. No. 17,467,680 filed on 2021 Sep. 7, was a continuation-in-part of U.S. patent application Ser. No. 17,466,497 filed on 2021 Sep. 3, was a continuation-in-part of U.S. patent application Ser. No. 17,353,652 filed on 2021 Jun. 21, was a continuation-in-part of U.S. patent application Ser. No. 17,220,002 filed on 2021 Apr. 1, was a continuation-in-part of U.S. patent application Ser. No. 17,214,827 filed on 2021 Mar. 27, was a continuation-in-part of U.S. patent application Ser. No. 17,211,446 filed on 2021 Mar. 24, was a continuation-in-part of U.S. patent application Ser. No. 16,693,267 filed on 2019 Nov. 23, and was a continuation-in-part of U.S. patent application Ser. No. 16,660,929 filed on 2019 Oct. 23.

U.S. patent application Ser. No. 17,220,002 was a continuation-in-part of U.S. patent application Ser. No. 17,214,827 filed on 2021 Mar. 27. U.S. patent application Ser. No. 17,220,002 was a continuation-in-part of U.S. patent application Ser. No. 17,211,446 filed on 2021 Mar. 24. U.S. patent application Ser. No. 17,220,002 claimed the priority benefit of U.S. provisional patent application Ser. No. 63,119,774 filed on 2020 Dec. 1. U.S. patent application Ser. No. 17,220,002 was a continuation-in-part of U.S. patent application Ser. No. 16,693,267 filed on 2019 Nov. 23. U.S. patent application Ser. No. 17,220,002 was a continuation-in-part of U.S. patent application 16,660,929 filed on 2019 Oct. 23.

U.S. patent application Ser. No. 16,693,267 was a continuation-in-part of U.S. patent application Ser. No. 16,660,929 filed on 2019 Oct. 23. U.S. patent application Ser. No. 16,693,267 claimed the priority benefit of U.S. provisional patent application Ser. No. 62,794,609 filed on 2019 Jan. 19. U.S. patent application Ser. No. 16,693,267 claimed the priority benefit of U.S. provisional patent application Ser. No. 62,794,607 filed on 2019 Jan. 19. U.S. patent application Ser. No. 16,693,267 was a continuation-in-part of U.S. patent application Ser. No. 16,541,241 filed on 2019 Aug. 15. U.S. patent application Ser. No. 16,693,267 was a continuation-in-part of U.S. patent application Ser. No. 15,865,822 filed on 2018 Jan. 9 which issued as U.S. Pat. No. 10,716,573 on 2020 Jul. 21. U.S. patent application Ser. No. 16,693,267 was a continuation-in-part of U.S. patent application Ser. No. 15,861,482 filed on 2018 Jan. 3.

U.S. patent application Ser. No. 16,660,929 claimed the priority benefit of U.S. provisional patent application 62,794,609 filed on 2019 Jan. 19. U.S. patent application Ser. No. 16,660,929 claimed the priority benefit of U.S. provisional patent application Ser. No. 62,794,607 filed on 2019 Jan. 19. U.S. patent application 16,660,929 was a continuation-in-part of U.S. patent application Ser. No. 16,541,241 filed on 2019 Aug. 15. U.S. patent application Ser. No. 16,660,929 was a continuation-in-part of U.S. patent application Ser. No. 15,865,822 filed on 2018 Jan. 9 which issued as U.S. Pat. No. 10,716,573 on 2020 Jul. 21. U.S. patent application Ser. No. 16,660,929 was a continuation-in-part of U.S. patent application Ser. No. 15,861,482 filed on 2018 Jan. 3.

U.S. patent application Ser. No. 165,41,241 claimed the priority benefit of U.S. provisional patent application 62,794,609 filed on 2019 Jan. 19. U.S. patent application Ser. No. 16,541,241 claimed the priority benefit of U.S. provisional patent application Ser. No. 62,794,607 filed on 2019 Jan. 19. U.S. patent application Ser. No. 16,541,241 claimed the priority benefit of U.S. provisional patent application Ser. No. 62,720,173 filed on 2018 Aug. 21. U.S. patent application Ser. No. 16,541,241 was a continuation-in-part of U.S. patent application Ser. No. 15,865,822 filed on 2018 Jan. 9 which issued as U.S. Pat. No. 10,716,573 on 2020 Jul. 21

U.S. patent application Ser. No. 15,865,822 claimed the priority benefit of U.S. provisional patent application 62,589,754 filed on 2017 Nov. 22. U.S. patent application Ser. No. 15,865,822 claimed the priority benefit of U.S. provisional patent application Ser. No. 62,472,519 filed on 2017 Mar. 16. U.S. patent application Ser. No. 15,861,482 claimed the priority benefit of U.S. provisional patent application Ser. No. 62,589,754 filed on 2017 Nov. 22. U.S. patent application Ser. No. 15,861,482 claimed the priority benefit of U.S. provisional patent application Ser. No. 62,472,519 filed on 2017 Mar. 16. U.S. patent application Ser. No. 15,861,482 claimed the priority benefit of U.S. provisional patent application Ser. No. 62,444,860 filed on 2017 Jan. 11.

The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field Of Invention

This invention relates to aneurysm occlusion devices and methods.

### INTRODUCTION

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain. Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Even among those who survive, approximately one-half suffer significant and permanent deterioration of brain function. Better alternatives for cerebral aneurysm treatment are needed.

### REVIEW OF THE RELEVANT ART

U.S. patent application publication 20150272589 (Lorenzo, Oct. 1, 2015, “Aneurysm Occlusion Device”) discloses a tubular structure with a control ring. U.S. Pat. No. 10,327,781 (Divino et al., Jun. 25, 2019, “Occlusive Devices”), U.S. Pat. No. 11,690,628 (Bardsley et al., Jul. 4, 2023, “Occlusive Devices”), U.S. Pat. No. 11,786,253 (Divino et al., Oct. 17, 2023, “Occlusive Devices”), and U.S. Pat. No. 12,193,675 (Divino et al., Jan. 14, 2025, “Occlusive Devices”) disclose expandable embolic structures with specific shapes and/or porosities. U.S. patent application publication 20190192168 (Lorenzo et al., Jun. 27, 2019, “Aneurysm Device and Delivery Method”) discloses a self-expanding braid which slides in a catheter in a collapsed state.

U.S. patent application publication 20190307546 (Aguilar et al., Oct. 10, 2019, “Embolic Device with Improved Neck Coverage”) discloses an embolic structure with a spiral shape. U.S. Pat. No. 10,653,425 (Gorochow et al., May 19, 2020, “Layered Braided Aneurysm Treatment Device”) discloses a tubular braid with a first segment from an open end to a first inversion, a second segment from the first inversion to a second inversion, and a third segment from the second inversion to a pinched end. U.S. patent application publication 20210128160 (Li et al., May 6, 2021, “Systems and Methods for Treating Aneurysms”) discloses a device comprising an expandable braid and embolic elements.

U.S. patent application publication 20210128167 (Patel et al., May 6, 2021, “Systems and Methods for Treating Aneurysms”) discloses an elongate tubular member with an engagement member which is removably coupled to a proximal hub. U.S. patent application publication 20210128169 (Li et al., May 6, 2021, “Devices, Systems, and Methods for Treatment of Intracranial Aneurysms”) discloses a method for deforming a shape of an occlusive member by inserting an embolic element between the occlusive member and an aneurysm wall. U.S. Pat. No. 11,071,551 (Garza et al., Jul. 27, 2021, “Flow Attenuation Device”) discloses an embolic device whose porosity varies along the length of the device.

U.S. patent application publication 20220031334 (Aguilar, Feb. 3, 2022, “Expandable Devices for Treating Body Lumens”) and U.S. Pat. No. 12,161,344 (Aguilar, Dec. 10, 2024, “Expandable Devices for Treating Body Lumens”) disclose an expandable mesh including an outer mesh and an inner mesh within the outer mesh. U.S. patent application publication 20230016312 (Xu et al., Jan. 19, 2023, “Aneurysm Treatment with Pushable Implanted Braid”) discloses a braided implant with a retractable dual proximal layer. U.S. patent application publication 20230277184 (Rashidi et al., Sep. 7, 2023, “Occlusive Devices with Thrombogenic Inserts”) discloses an insert between the upper and lower walls within an expandable mesh.

U.S. patent application publication 20240032941 (Shimizu et al., Feb. 1, 2024, “Embolic Material Delivery Device and Related Technology”) discloses an elongate conduit body defining an axial lumen through which a liquid embolic material is conveyed to an aneurysm. U.S. patent application publication 20240050099 (Pecor et al., Feb. 15, 2024, “Occlusive Devices for Treating Vascular Defects and Associated Systems and Methods”) discloses a mesh comprising at least two mesh layers and a membrane between the layers. U.S. patent application publication 20240065702 (Ogawa et al., Feb. 29, 2024, “Embolization Device”) discloses an outer tube having a distal end and a proximal end and a basket in a lumen of the outer tube.

U.S. patent application publication 20240075565 (Li et al., Mar. 7, 2024, “Systems and Methods for Treating Aneurysms”) discloses an electrolytically-corrodible core wire having a proximal portion, a distal portion, and a detachment zone between the proximal portion and the distal portion. U.S. patent application publication 20240099723 (Schabert et al., Mar. 28, 2024, “Intrasaccular Neck Bridging Device”) discloses a braided mesh body and at least one pinch member. U.S. patent application publication 20240206879 (Kandala et al., Jun. 27, 2024, “Occlusive Devices with Spiral Struts for Treating Vascular Defects”) discloses a plurality of spiral struts which are coupled to an anchor structure.

U.S. Pat. No. 12,029,431 (Griffin, Jul. 9, 2024, “Occlusion Device”) discloses an occlusion device for intrasaccular implantation with a substantially solid marker and a low profile resilient mesh body attached to a distal end of the marker. U.S. Pat. No. 12,053,182 (Aboytes et al., Aug. 6, 2024, “Devices and Methods for the Treatment of Vascular Defects”) discloses an expandable implant which can be moved from a first configuration in which a first portion and a second portion are substantially linearly aligned to a second configuration in which the second portion overlaps the first portion. U.S. Pat. No. 12,059,156 (Mayer et al., Aug. 13, 2024, “Devices for Treating Vascular Malformations”) discloses an apparatus with an orifice-section curve that winds at least 2.5 turns around an orifice-section central axis at a changing distance from the orifice-section central axis.

U.S. Pat. No. 12,070,220 (Dholakia et al., Aug. 27, 2024, “Devices Having Multiple Permeable Shells for Treatment of Vascular Defects”) discloses a plurality of permeable shells which are connected by a plurality of coils, wherein each coil connects at least one pair of permeable shells. U.S. Pat. No. 12,076,022 (Griffin, Sep. 3, 2024, “Occlusion Device”) discloses a continuous compressible mesh structure comprising axial mesh carriages configured end to end, wherein each end of each carriage is a pinch point in the continuous mesh structure. U.S. Pat. No. 12,082,821 (Marchand et al., Sep. 10, 2024, “Filamentary Devices for Treatment of Vascular Defects”) discloses a permeable shell having a radially-constrained elongated state within a catheter, an expanded state with a longitudinally-shortened configuration, and a plurality of elongate filaments that are woven together to form a mesh.

U.S. Pat. No. 12,096,940 (Hewitt et al., Sep. 24, 2024, “Filamentary Devices for Treatment of Vascular Defects”) discloses a self-expanding resilient permeable shell having a radially constrained state and an expanded state with a globular, axially shortened configuration. U.S. Pat. No. 12,102,327 (Pereira et al., Oct. 1, 2024, “Systems and Methods for Treating Aneurysms”) discloses an occlusion element comprising an inverted mesh tube with an outer layer and an inner layer, wherein the outer layer transitions to the inner layer at an inversion fold located at or adjacent the distal end of the occlusion element and the inversion fold defines an inner diameter. U.S. patent application publication 20240341769 (Bai et al., Oct. 17, 2024, “Intrasaccular Occlusive Devices Comprising Copper Materials”) discloses a mesh having a low-profile configuration for delivery through a catheter to an aneurysm sac and an expanded configuration for implantation in the aneurysm sac.

U.S. patent application publication 20240358376 (Khenansho, Oct. 31, 2024, “Systems and Methods for Occluding Vascular Defects”) discloses an occlusive device whose height is less than the height of an aneurysm such that a space exists between a distal surface of the occlusive member and a dome of the aneurysm. U.S. patent application publication 20240366226 (Gorochow et al., Nov. 7, 2024, “Braided Aneurysm Treatment Device with Flexible Inversion Region”) discloses a tubular braid with an open end, a pinched end, and a predetermined shape. U.S. patent application publication 20240366227 (Tran et al., Nov. 7, 2024, “Devices for Treatment of Vascular Defects”) discloses a permeable shell with an unrestrained preset configuration comprising a dome portion and a brim portion.

U.S. patent application publication 20240382208 (Tafti, Nov. 21, 2024, “Device for Vascular Occlusion and Methods of Use Thereof”) discloses arcuately-curved coils. U.S. Pat. No. 12,150,871 (Ruvalcaba et al., Nov. 26, 2024, “Occlusive Device”) discloses an aneurysm embolization device with an atraumatic tip portion extending from it. U.S. patent application publication 20240398412 (Zaidat et al., Dec. 5, 2024, “Systems and Methods for Treating Aneurysms”) discloses an inverted mesh tube having an outer layer and an inner layer, wherein the outer layer transitions to the inner layer at an inversion fold located at or adjacent the distal end of the occlusion element, and the inversion fold defines an inner diameter.

U.S. patent application publication 20250032121 (Rashidi et al., Jan. 30, 2025, “Systems and Methods for Treating Aneurysms”) discloses an occlusive implant, wherein expanding an expandable member is a balloon. U.S. patent application publication 20250041084 (Monstadt et al., Feb. 6, 2025, “Implant for Treating Aneurysms in the Area of Bifurcations”) discloses an implant with at least two branching tubular sections. U.S. patent application publication 20250049592 (Ruvalcaba et al., Feb. 13, 2025, “Occlusive Device”) discloses an expandable component and an atraumatic tip portion extending from it.

U.S. Pat. No. 12,256,936 (Li et al., Mar. 25, 2025, “Devices, Systems, and Methods for Treatment of Intracranial Aneurysms”) discloses a method for deforming the shape of an occlusive member by introducing an embolic element to a space between the occlusive member and an aneurysm wall. U.S. patent application publication 20250120654 (Salant et al., Apr. 17, 2025, “Implantable Medical Device with Sensing and Communication Functionality”) discloses an implantable system for monitoring a vascular structure with a sensor which transmits blood flow data. U.S. Pat. No. 12,303,136 (Mauger et al., May 20, 2025, “System and Methods for Embolized Occlusion of Neurovascular Aneurysms”) discloses an occlusion device which transitions between a two-dimensional configuration and a three-dimensional configuration.

U.S. patent application publication 20250169824 (Li et al., May 29, 2025, “Devices, Systems, and Methods for Treatment of Intracranial Aneurysms”) discloses a method for deforming the shape of the occlusive member by introduction of an embolic element to a space between the occlusive member and an aneurysm wall. U.S. patent application publication 20250176967 (Carrillo, Jun. 5, 2025, “Aneurysm Treatment Device and Associated Systems and Methods of Use”) discloses an aneurysm treatment device with a tip portion, a body portion, and a base portion.

### SUMMARY OF THE INVENTION

This invention is an intrasaccular aneurysm occlusion device with an expandable mesh which is inserted and expanded within an aneurysm sac. The expandable mesh can be steered in a selected direction and/or expanded in a radially-asymmetric manner by: moving a joint on a catheter, sliding sections longitudinally on the catheter, selectively pulling or pushing wires or cords attached to different locations on the mesh, selectively detaching constraints on the mesh, or selectively filling chambers in the mesh with embolic material. In an example, there can a plurality of non-central openings with remotely-controllable valves in the expandable mesh through which embolic material can be inserted into the aneurysm sac. In an example, the expandable mesh can be expanded into a single-layer convex shape within the aneurysm sac and then collapsed into a two-layer concave shape.

### BRIEF INTRODUCTION TO THE FIGURES

FIG. 1 shows an expandable mesh which is steered by a radially-asymmetric catheter.

FIG. 2 shows an expandable mesh which is steered by a coaxial catheter with movable inner and outer layers.

FIG. 3 shows an expandable mesh which is steered by a catheter with a rotational joint.

FIG. 4 shows an expandable mesh which is steered by a catheter with a longitudinally-sliding sections.

FIG. 5 shows an expandable mesh which is steered by a catheter with a pleated joint.

FIG. 6 shows an expandable mesh which is steered by a catheter with an annular array of pistons.

FIG. 7 shows a steerable catheter with a balloon on one side of a distal end portion.

FIG. 8 shows a steerable catheter with balloons on two sides of a distal end portion.

FIG. 9 shows an expandable mesh which is steered by a pivoting or rotating joint.

FIG. 10 shows a coil loop which is inserted into an aneurysm sac from a dual catheter.

FIG. 11 shows an expandable mesh on a coil loop.

FIG. 12 shows a longitudinal series of expandable meshes with connected wires or cords.

FIG. 13 shows a longitudinally multi-lobed mesh with connected wires or cords.

FIG. 14 shows an occlusion device with a toroidal mesh and a dual catheter.

FIG. 15 shows an expandable mesh with outer-connected wires or cords.

FIG. 16 shows an expandable mesh with an outer-connected wire loop.

FIG. 17 shows an expandable mesh with inner-connected wires or cords.

FIG. 18 shows an expandable mesh with an array of undulating radial wires around a hub.

FIG. 19 shows an expandable mesh with an array of undulating and straight radial wires.

FIG. 20 shows an expandable mesh with a circumferential constraint with detachable connections.

FIG. 21 shows an expandable mesh with proximal-to-distal constraints with detachable connections.

FIG. 22 shows an expandable mesh with radial constraints with detachable connections.

FIG. 23 shows an expandable mesh with radial constraints and a hub with detachable connections.

FIG. 24 shows an expandable mesh with an off-center opening.

FIG. 25 shows an expandable mesh with a rotatable bowl with an off-center opening.

FIG. 26 shows an expandable mesh with a plurality of controllable off-center openings.

FIG. 27 shows a radially-asymmetric expandable mesh.

FIG. 28 shows a device comprising two expandable meshes and a dual catheter.

FIG. 29 shows a multi-layer expandable mesh with fluid-filled chambers between layers.

FIG. 30 shows a multi-layer expandable mesh wherein embolic material is inserted between layers.

FIG. 31 shows a coil loop, wherein one loop branch is rotated relative to the other loop branch.

FIG. 32 shows a string-of-pearls loop, wherein one loop branch is rotated relative to the other loop branch.

FIGS. 33 through 35 show three successive views of a toroidal mesh and embolic coils inserted through the center of the mesh.

FIGS. 36 through 38 show three successive views of a longitudinal series of coil loops.

FIGS. 39 through 41 show three successive views of a longitudinal series of convex meshes.

FIGS. 42 through 44 show three successive views of a flexible net or mesh with a circumferential band or ring which is filled with embolic material.

FIG. 45 shows a mesh which is expanded in a radially-asymmetric manner by filling a mesh chamber with embolic material.

FIG. 46 shows a mesh which is expanded in a radially-asymmetric manner by an expandable chamber within the mesh.

FIGS. 47 through 50 shows four successive views of an expandable chamber which is radially-expanded into a convex shape within an aneurysm sac and then collapsed into a concave shape by the accumulation of embolic material.

FIGS. 51 through 54 show four views of an expandable mesh which is shaped like a revolution of the perimeter of a yin (or yang) symbol.

FIG. 55 shows an expandable mesh which is steered and/or expanded in a radially-asymmetric manner by activation of piezoelectric members.

FIG. 56 shows an expandable mesh which is steered and/or expanded in a radially-asymmetric manner by pulling or pushing wires.

FIG. 57 shows an expandable mesh, wherein there are fluid-filled chambers in the catheter.

FIG. 58 shows an expandable mesh, wherein an end portion of a catheter protrudes radially inward.

### DETAILED DESCRIPTION OF THE FIGURES

In an example, an intrasaccular aneurysm occlusion device can comprise: an expandable mesh that is configured to be inserted into an aneurysm sac; embolic members or material that are inserted into the aneurysm sac; and a catheter through which the expandable mesh and/or the embolic members or material are delivered to the aneurysm sac. In an example, the device can further comprise a distal end portion of the catheter and a pivoting and/or rotating joint between the distal end portion and the rest of the catheter, wherein the expandable mesh is steered in a selected direction and/or expanded in a radially-asymmetric manner by pivoting and/or rotating the joint. In an example, the device can further comprise a first partially-circumferential section of the catheter and a second partially-circumferential section of the catheter, wherein the expandable mesh is steered in a selected direction and/or expanded in a radially-asymmetric manner by sliding the first partially-circumferential section relative to the second partially-circumferential section.

In an example, the device can further comprise a plurality of wires or cords which are attached to different locations on the expandable mesh, wherein the expandable mesh is steered in a selected direction and/or expanded in a radially-asymmetric manner by selectively pulling, pushing, or rotating a subset of one or more wires or cords in the plurality of wires or cords. In an example, the device can further comprise a plurality of detachable constraints on the expandable mesh which constrain radial expansion of the expandable mesh, wherein the expandable mesh is steered in a selected direction and/or expanded in a radially-asymmetric manner by selective detachment of a subset of one or more of the detachable constraints.

In an example, an expandable mesh can have two or more layers, wherein the expandable has gaps or chambers between mesh layers, and wherein the expandable mesh is steered in a selected direction and/or expanded in a radially-asymmetric manner by selectively filling a subset of one or more of the gaps or chambers with embolic members or material. In an example, there can be a plurality of openings with valves in the expandable mesh, wherein selected valves can be opened or closed by a device operator, and wherein embolic members or material are inserted into the aneurysm sac through a subset of one or more selected openings whose valves have been opened by the device operator. In an example, the plurality of openings can include a plurality of non-central openings. In an example, an expandable mesh can be a composite mesh which further comprises a proximal-to-distal series of connected expandable meshes. In an example, an expandable mesh can comprise a proximal-to-distal series of lobes or undulations created by radially-constraining a continuous tubular mesh at multiple longitudinal locations.

In an example, an intrasaccular aneurysm occlusion device can comprise: a flexible net or mesh that is inserted into an aneurysm sac; an expandable band or ring that is inserted into the aneurysm sac, wherein the expandable band or ring spans a circumference of the flexible net or mesh; embolic members or material which are inserted into the flexible net or mesh; and a catheter through which the flexible net or mesh, the expandable band or ring, and/or the embolic members or material are delivered to the aneurysm sac. In an example, an expandable band or ring can be outside the flexible net or mesh. In an example, an expandable band or ring can be inside the flexible net or mesh. In an example, an expandable band or ring can have a proximal-to-distal width which is between 5% and 20% of a proximal-to-distal distance from a neck of the aneurysm to the peak of the dome of the aneurysm. In an example, there is an opening in the flexible net or mesh through which embolic members or material are inserted into the flexible net or mesh.

In an example, an intrasaccular aneurysm occlusion device can comprise: an expandable mesh that is inserted into an aneurysm sac; wherein the expandable mesh is radially expanded within the aneurysm sac at a first time into a first configuration having a convex shape; wherein the expandable mesh is collapsed, compressed, and/or inverted within the aneurysm sac at a second time into a second configuration having a concave shape; wherein the expandable mesh further comprises a flexible annular section or band which spans a circumference of the expandable mesh; wherein the flexible annular section of band is more flexible and/or elastic than the rest of the expandable mesh; wherein a first portion of the expandable mesh is distal relative to the flexible annular section or band in the first configuration; wherein a second portion of the expandable mesh is proximal relative to the flexible annular section or band in the first configuration; wherein the first portion of the expandable mesh is collapsed, compressed, and/or inverted into the second portion of the expandable mesh when the expandable mesh is changed from the first configuration to the second configuration; wherein both the first portion of the expandable mesh and the second portion of the expandable mesh are proximal relative to the flexible annular section or band in the second configuration; embolic members or material that are inserted into portions of the aneurysm sac which are distal relative to the expandable mesh; wherein pressure from accumulation of the embolic members or material collapses, compresses, and/or inverts the first portion of the expandable mesh into the second portion of the expandable mesh; and one or more catheters which deliver the expandable mesh and/or the embolic members or material to the aneurysm sac.

In an example, a flexible annular section or band can comprise between 2% and 10% of the expandable mesh. In an example, a flexible annular section or band can comprise between 5% and 20% of the expandable mesh. In an example, mesh components in the flexible annular section can be more flexible, more elastic, thinner, smaller, and/or more-sparsely woven or braided than mesh components in the rest of the expandable mesh. In an example, mesh components can be woven or braided wires, tubes, or filaments.

In an example, an expandable mesh can be a neck bridge. In an example, an expandable mesh can be a wire mesh. In an example, an expandable mesh can be made by braiding or weaving metal wires or tubes. In an example, an expandable mesh can be made by radially constraining a mesh tube at different locations by crimping or pinching. In an example, a distal portion (e.g. the distal third or half) of a net or mesh can be more elastic and/or stretchable than a proximal portion (e.g. the proximal third or half) of the net or mesh. In an example, an expandable mesh can be a braid.

In an example, an expandable mesh can be a convex (e.g. globular, spherical, ellipsoidal, or oblate spherical) mesh. In an example, an expandable mesh can have a convex polyhedron shape. In an example, an expandable mesh can have a hemispherical or hemi-elliptical shape. In another example, an expandable mesh can have a Saturn shape (e.g. generally globular with a central circumferential ring or band). In an example, an expandable mesh can expand into a bowl or cup shape within an aneurysm sac. In another example, an expandable mesh can expand into a disk shape within an aneurysm sac. In an example, an expandable mesh can expand into a globular shape within an aneurysm sac. In an example, an expandable mesh can expand into a multi-lobed shape within an aneurysm sac. In an example, an expandable mesh can expand into a torus shape within an aneurysm sac. In an example, an expandable mesh can expand into an hourglass shape within an aneurysm sac.

In an example, a device operator can cause a multi-layer (concave) mesh to become radially-asymmetric by filling one chamber between the layers of the mesh more than another chamber between the layers of the mesh. In an example, a device operator can steer a multi-layer mesh in a selected direction, at a selected angle, or into a selected orientation by selectively filling a first gap between layers on a first (e.g. right or left) side of the mesh with embolic members and/or material more than a second gap between layers on a second (e.g. left or right) side of the mesh. In an example, an expandable mesh can be a dual-layer mesh. In another example, a device operator can steer a multi-layer mesh in a selected direction, at a selected angle, and/or with a selected orientation by selectively and differentially filling different radial portions of a gap between layers of the mesh.

In an example, a convex (e.g. globular) expandable mesh can have a flexible annular section around its (central) circumference, wherein a distal portion (e.g. distal half) of the mesh is collapsed, compressed, and/or inverted around the annular section into a proximal portion (e.g. proximal half) of the mesh by a wire attached to the distal portion which is pulled by a device operator. In another example, an expandable mesh can be changed from a convex (e.g. globular) shape to a concave (e.g. bowl) shape by collapsing, compressing, and/or inverting a distal portion of the mesh into a concavity of a proximal portion of the mesh by pulling a wire attached to the distal portion. In an example, an expandable mesh can expand into a globular (e.g. spherical, ellipsoidal, or oblate spheroidal) shape in an aneurysm sac and then be collapsed, compressed, and/or inverted into a concave (e.g. bowl, cup, hemispherical, hemi-ellipsoidal) shape by pressure from the accumulation of embolic members and/or material in the aneurysm sac.

In an example, a device can comprise a convex distal mesh and a proximal mesh, wherein at least 45% of the (proximal-to-distal axis of) the distal mesh is nested within a concavity of a concave proximal mesh. In an example, a device can comprise a convex mesh and a concave mesh, wherein the concave mesh is nested within the proximal half of the convex mesh. In an example, a device can comprise a distal mesh and a proximal mesh, wherein at least 80% of the (proximal-to-distal axis of) the distal mesh is nested within a concavity of a proximal mesh.

In an example, a device can comprise a distal mesh which is nested within a concavity of a proximal mesh, wherein the distal mesh is more porous than the proximal mesh. In an example, a device can comprise a distal mesh which is nested within a concavity of a proximal mesh, wherein the distal mesh has a lower durometer and/or Shore value than the proximal mesh. In another example, a device can comprise a distal mesh which is nested within a concavity of a proximal mesh, wherein the distal mesh is made with polymer material and the proximal mesh is made with metal.

In an example, a device can comprise a multi-layer mesh, wherein the multi-layer mesh comprises a convex inner mesh which is inside the proximal half of a convex outer mesh. In an example, a device can comprise a distal flexible net or mesh which is nested within the concavity of a concave mesh, wherein the distal flexible net or mesh is more flexible than the concave mesh. In an example, a device can comprise a distal flexible net or mesh which is nested within the concavity of a concave mesh, wherein embolic members and/or material is inserted into the distal flexible net or mesh. In an example, a flexible net or mesh can be made by 3D printing.

In an example, a device can comprise a proximal-to-distal plurality (e.g. series, sequence, or stack) of expandable meshes which is inserted into an aneurysm sac, wherein central proximal-to-distal axes of the meshes are colinear. In an example, a device can comprise a proximal-to-distal plurality (e.g. series, sequence, or stack) of expandable meshes which is inserted into an aneurysm sac, wherein the meshes are connected by a flexible longitudinal member (e.g. wire, cord, suture, string, or coil) which passes through their centers. In another example, a device can comprise a proximal-to-distal plurality (e.g. series, sequence, or stack) of expandable meshes which is inserted sequentially (e.g. one at a time) into an aneurysm sac.

In an example, a device can comprise a proximal-to-distal plurality (e.g. series, sequence, or stack) of expandable meshes which is inserted into an aneurysm sac, wherein the expandable meshes are connected to each other by a flexible longitudinal member (e.g. wire, cord, string, suture, or coil), wherein expandable meshes are inserted and distances between them are adjusted sequentially (e.g. one at a time) by pulling, pushing, and/or rotating the flexible longitudinal member. In an example, a device can comprise a proximal-to-distal plurality (e.g. series, sequence, or stack) of expandable meshes are made from the same continuous piece (e.g. from a tubular mesh). In an example, a device can comprise a proximal-to-distal plurality (e.g. series, sequence, or stack) of expandable meshes which is inserted into an aneurysm sac, wherein some of the meshes have convex shapes and some of the meshes have concave shapes.

In an example, a device can comprise a proximal-to-distal plurality (e.g. series, sequence, or stack) of expandable meshes which is inserted into an aneurysm sac, wherein a distal mesh in the series is larger than a proximal mesh in the series. In another example, a device can comprise a proximal-to-distal plurality (e.g. series, sequence, or stack) of expandable meshes which is inserted into an aneurysm sac, wherein the plurality includes a central or middle mesh between a proximal mesh and a distal mesh, and wherein the central or middle mesh is smaller than the proximal mesh or the distal mesh. In an example, a device can comprise a proximal-to-distal plurality (e.g. series, sequence, or stack) of expandable meshes which is inserted into an aneurysm sac, wherein the expandable meshes have torus shapes. In another example, a device can comprise a proximal-to-distal plurality (e.g. series, sequence, or stack) of expandable meshes which is inserted into an aneurysm sac, wherein the expandable meshes have bowl or cup shapes. In an example, central axes of expandable meshes in a series can be linearly aligned.

In an example, distal expandable meshes in a longitudinal series (e.g. series, sequence, or stack) of expandable meshes can be farther apart than proximal expandable meshes in the series. In an example, distal expandable meshes in a longitudinal series (e.g. series, sequence, or stack) of expandable meshes can be more elastic and/or flexible than proximal expandable meshes in the series. In an example, distal expandable meshes in a longitudinal series (e.g. series, sequence, or stack) of expandable meshes can be more convex than proximal expandable meshes in the series.

In an example, a device can comprise a longitudinal series (e.g. series, sequence, or stack) of expandable meshes which is inserted into an aneurysm sac, wherein there are one or more flexible longitudinal members (e.g. wires, sutures, cords, strings, or coils) connected to the expandable meshes, and wherein pulling, pushing, or rotating a subset of the longitudinal members causes the longitudinal series to expand in a radially-asymmetric manner.

In an example, a flexible annular section around a circumference of an expandable mesh can span the widest circumference of the mesh. In an example, an annular section around a central circumference of an expandable mesh can have a lower durometer and/or Shore value than the rest of the mesh. In another example, an expandable mesh can comprise an annular section around a (central) conference of the mesh, wherein the annular section is more flexible, more elastic, and/or thinner than the rest of the mesh] so that a distal portion (e.g. distal half) of the mesh can collapse and/or invert into a proximal portion (e.g. proximal half) of the mesh when pressure is applied to the distal portion.

In an example, a band or ring around an expandable mesh can be made by weaving and/or braiding wires, tubes, and/or filaments. In an example, a device can comprise a band or ring which spans between 2% and 10% of the surface area of a net or mesh. In an example, a device can further comprise a band or ring around a (central) circumference of a net or mesh, wherein the band or ring is less flexible, less elastic, less compressible, and/or more resilient than the net or mesh. In an example, a net or mesh can be made from one or more polymers and a band or ring around a circumference of the net or mesh can be made from one or more metals. In an example, an expandable mesh can include a band or ring with multiple sections, wherein selective detachment of one or more of the sections by the device operator causes the mesh to expand in a radially-asymmetric manner. In another example, an expandable mesh can include a multi-section band or ring which radially-constrains the mesh, wherein selective detachment of one or more sections of the band or ring causes the mesh to expand in a radially-asymmetric manner.

In an example, an expandable mesh can have multiple lobes and/or undulations with colinear central axes. In another example, an expandable mesh can have multiple lobes or undulations, wherein a distal lobe or undulation is at least 20% larger than a proximal lobe or undulation. In an example, an expandable mesh can have multiple longitudinal lobes and/or undulations. In an example, an expandable mesh can have sinusoidal longitudinal lobes or undulations.

In an example, a device can include a plurality of detachable connections on one or more radial constraints, wherein selective detachment of a subset of one or more of the detachable connections causes asymmetric radial expansion of an expandable mesh. In an example, a device can include a radial array of detachable constraints which span a circumference of an expandable mesh. In an example, a device can include at least three detachable radial constraints on an expandable mesh. In another example, a device can include one or more radial constraints on an expandable mesh, wherein selective detachment of one or more sections of the radial constraints steers the mesh in a selected direction, at a selected angle, or into a selected orientation.

In an example, a device can include one or more selectively-detachable constraints on an expandable mesh, wherein a device operator can selectively detach one or more of the constraints to cause the mesh to expand in an asymmetric manner. In another example, a device can include one or more selectively-detachable circumferential constraints on an expandable mesh, wherein a device operator can selectively detach one or more of the circumferential constraints to cause the mesh to expand in a radially-asymmetric manner. In an example, a device can include one or more selectively-detachable constraints on an expandable mesh, wherein a device operator can selectively detach one or more of the constraints to steer the mesh in a selected direction, at a selected angle, or into a selected orientation.

In an example, a device can include one or more selectively-detachable circumferential constraints on an expandable mesh, wherein a device operator can selectively detach one or more of the circumferential constraints to steer the mesh in a selected direction, at a selected angle, or into a selected orientation. In an example, a device operator can selectively detach one or more (sections of) radial or longitudinal constraints on an expandable mesh in order to cause the mesh to expand in an asymmetric manner. In an example, a device operator can selectively detach one or more (sections of) radial or longitudinal constraints on an expandable mesh in order to cause the mesh to expand in a radially-asymmetric manner.

In an example, a device can comprise a circumferential constraint (e.g. ring or band) around a circumference of an expandable mesh. In another example, a device can comprise a circumferential constraint (e.g. ring or band) with a plurality of detachable sections around a distal circumference of a concave expandable mesh, wherein a device operator can cause the expandable mesh to expand in an asymmetric manner by selectively detaching a subset of the detachable sections. In an example, a device can comprise a detachable circumferential constraint (e.g. ring or band) around a distal circumference of an expandable mesh. In another example, a device can comprise one or more circumferential constraints (e.g. rings or bands) around one or more circumferences of an expandable mesh.

In an example, a device can further comprise an electroconductive pathway (e.g. wire) which transmits and/or conducts electrical energy to a detachable connection. In an example, a device can include a wire which detaches a detachable connection on an expandable mesh when the wire is pushed or pulled. In an example, a device operator can control the radially-asymmetric expansion of an expandable mesh. In an example, a device operator can control the radially-asymmetric expansion of an expandable mesh in order to steer the mesh in a selected direction, at a selected angle, or into a selected orientation. In another example, a first side of an expandable mesh can expand more rapidly and/or to a greater extent than a second side of the mesh.

In an example, a device which enables control over asymmetric expansion of an expandable mesh in an aneurysm sac can enable a device operator to fill an aneurysm sac more thoroughly, completely, and/or quickly than a device which does not enable asymmetric expansion an expandable mesh, especially if the aneurysm is irregularly shaped. In another example, a device which enables steering an expandable mesh in a selected direction, at a selected angle, and/or into a selection orientation can enable a device operator to fill an irregularly-shaped aneurysm sac more thoroughly, completely, and/or quickly than a device which does not enable steering an expandable mesh.

In an example, a device can further comprise one or more actuators which push, pull, or rotate flexible longitudinal members to steer a mesh in a selected direction, at a selected angle, or with a selected orientation. In an example, a device can include a plurality of flexible longitudinal members (e.g. wires) which are connected at different locations to an expandable mesh, wherein the expandable mesh is steered in a selected direction, at a selected angle, or into a selected orientation when a subset of the flexible longitudinal members are selectively pulled, pushed, and/or rotated by the device operator. In an example, a device can include a plurality of flexible longitudinal members (e.g. wires) which are connected at different locations to an expandable mesh, wherein the expandable mesh expands in a selected radially-asymmetric manner when one or more of the flexible longitudinal members are pulled, pushed, and/or rotated.

In an example, a device can include a plurality of flexible longitudinal members (e.g. wires) which span the length of a catheter and are connected at different locations to an expandable mesh, wherein the expandable mesh expands in a selected radially-asymmetric manner when a subset of the flexible longitudinal members are selectively pulled, pushed, and/or rotated by the device operator. In another example, a device can include one or more flexible longitudinal members (e.g. wires) which span the length of a catheter and are connected to an expandable mesh, wherein the expandable mesh is steered in a selected direction, at a selected angle, or into a selected orientation when one or more of the flexible longitudinal members are pulled, pushed, and/or rotated.

In an example, a device can include one or more flexible longitudinal members (e.g. wires) which are connected to an expandable mesh, wherein the expandable mesh expands in a selected radially-asymmetric manner when one or more of the flexible longitudinal members are selectively pulled, pushed, and/or rotated by a device operator. In another example, a device operator can control asymmetric expansion of an expandable mesh by selectively pulling, pushing, and/or rotating one or more flexible longitudinal members (e.g. wires) which are connected to different locations on the mesh. In an example, a device operator can manually push, pull, or rotate flexible longitudinal members to control radially-asymmetric expansion of an expandable mesh.

In an example, a flexible longitudinal member can be a chain. In an example, a flexible longitudinal member can be an elastic cord. In an example, a flexible longitudinal member can travel through the inner lumen of a channel. In another example, flexible longitudinal piezoelectric members can be connected to different locations (e.g. to opposite sides) of an expandable mesh, wherein transmission of electricity to a selected subset of one or more of the piezoelectric members changes the lengths of the one or more piezoelectric members, causing the mesh to expand in an asymmetric manner and/or steering the mesh in a selected direction, at a selected angle, or with a selected orientation. In an example, a device can comprise an expandable mesh with multiple openings (e.g. including a central opening and multiple non-central openings), wherein a device operator can select which of the openings to use to insert expandable members and/or material through a selected opening into an aneurysm sac.

In an example, a device can comprise an expandable mesh with multiple potential opening locations (e.g. a central location and multiple non-central locations), wherein a device operator selects which of the potential opening locations to open to insert expandable members and/or material into an aneurysm sac, and wherein the device operator opens a selected opening location by applying light energy that location. In an example, an expandable mesh can have a plurality of adjustable openings, wherein a subset of one or more of these openings can be selectively opened by a device operator to insert embolic members and/or material through the expandable mesh. In an example, an opening in a multi-layer mesh through which embolic members and/or material are inserted can be formed by rotating a portion of the mesh to align an opening in a first layer of the mesh with an opening in a second layer of the mesh.

In an example, there can be a central opening and plurality of non-central openings in an expandable mesh through which embolic members and/or material are inserted into an aneurysm sac. In an example, there can be a plurality of openings on an expandable mesh, wherein there is a valve on each of the openings. In another example, there can be an off-center opening in an expandable mesh through which embolic members and/or material are inserted into an aneurysm sac. In an example, there can be an opening in an expandable mesh through which embolic members and/or material are inserted into an aneurysm sac. In another example, there can be one or more openings in an expandable mesh through which embolic members and/or material are inserted into an aneurysm sac, wherein the opening is larger than pores between mesh wires or strands elsewhere on the mesh.

In an example, a device operator can remotely control the operation of a valve in an opening through an expandable mesh using a butterfly valve. In an example, a device operator can remotely control the operation of a valve in an opening through an expandable mesh using a clamp. In an example, a device operator can remotely control the operation of a valve in an opening through an expandable mesh using a drawstring. In an example, a device operator can remotely control the operation of a valve in an opening through an expandable mesh using a globe valve. In an example, a device operator can remotely control the operation of a valve in an opening through an expandable mesh using a pinch valve.

In an example, a device operator can remotely control the operation of a valve in an opening through an expandable mesh using a pull cord. In another example, a device operator can remotely control the operation of a valve in an opening through an expandable mesh using a sliding valve. In an example, a device operator can remotely control the operation of a valve in an opening through an expandable mesh using a threaded valve. In another example, there can be a one-way valve in an opening through an expandable mesh through which embolic members and/or material can be inserted into an aneurysm sac. In an example, there can be a valve in an opening through an expandable mesh through which embolic members and/or material can be inserted into an aneurysm sac, wherein a device operator can remotely open or close the valve by pulling, pushing, or rotating a wire connected to the valve.

In an example, a proximal mesh can have a higher proportion of metal components than a distal mesh. In an example, an expandable mesh can be made from a thermoplastic polymer. In an example, an expandable mesh can be made with a polymer which has been doped or impregnated with metal particles. In another example, a flexible net or mesh can be made by laser-cutting holes in a globular metal structure. In an example, a flexible net or mesh can be made by 3D printing a polymer ink. In another example, a flexible net or mesh can be made from an elastomeric polymer. In an example, a flexible net or mesh can be formed from a combination of metal wires (or tubes) and polymer strands, filaments, and/or threads. In an example, one or more portions of a flexible net or mesh can be made from a radio-opaque material. In an example, one or more portions of an expandable mesh can be made from a radio-opaque material.

In an example, a device can include a catheter with a diagonal end which steers an expandable mesh in a selected direction, at a selected angle, or into a selected orientation. In an example, a device can include a catheter with a diagonally-cut end which causes an expandable mesh to expand in a radially-asymmetric manner. In an example, a device operator can use a catheter with a diagonal end to steer an expandable mesh in a selected direction, at a selected angle, or into a selected orientation. In an example, a device operator can use a catheter with a diagonally-cut end to control radially-asymmetric expansion of an expandable mesh.

In an example, a diagonally-cut end of a catheter can intersect the central longitudinal axis of the catheter at an acute angle (e.g. between 25 and 75 degrees). In an example, an end portion of a catheter can be curved like a piece of plumbing pipe. In an example, the angle of the end of a dual catheter can be changed by longitudinally sliding an inner catheter relative to an outer catheter, or vice versa, wherein one of the catheters has a radially-symmetric (e.g. straight cut) end and the other catheter has a radially-asymmetric (e.g. diagonally cut) end. In another example, a device can further comprise one or more actuators which move (e.g. rotated, pivot, and/or tilt) an end portion of a catheter to steer an expandable mesh in a selected direction, at a selected angle, and/or with a selected orientation.

In an example, a device can further comprise one or more solenoids which move (e.g. rotated, pivot, and/or tilt) an end portion of a catheter to steer an expandable mesh in a selected direction, at a selected angle, and/or with a selected orientation. In another example, an end portion of a catheter can be connected to the rest of the catheter by a joint, wherein the end portion is moved (e.g. rotated, pivoted, and/or tilted) by the joint in order to steer an expandable mesh in a selection direction as the mesh exits the catheter. In an example, an end portion of a catheter can be moved (e.g. rotated, pivoted, and/or tilted) in order to steer an expandable mesh in a selection direction as the mesh exits the catheter.

In an example, a device can include a movable (e.g. rotatable, pivoting, and/or tilting) joint between a radially-asymmetric (e.g. curved or diagonally-cut) end portion of catheter and the rest of the catheter, wherein the movable joint enables a device operator to steer the expandable mesh in a selected direction, at a selected angle, or into a selected orientation. In an example, a device operator can use one or more actuators to move a joint between an end portion of a catheter and the rest of the catheter. In an example, a device operator can use one or more hydraulic actuators (e.g. micropistons) to move a joint between a catheter and an expandable mesh. In an example, a joint between a catheter and an expandable mesh can be moved by one or more electromagnetic actuators (e.g. micromotors). In another example, a joint between an end portion of a catheter and the rest of the catheter can be moved by one or more actuators. In an example, a movable joint can be a pivoting joint. In another example, device can comprise an actuator which moves (e.g. pivots and/or rotates) the joint which, in turn, pivots and/or rotates an expandable mesh.

In an example, a device can a coaxial catheter, wherein there are one or more wires or cords between an inner catheter and outer catheter in the coaxial catheter, and wherein pulling or pushing the one or more wires or cords steers an expandable mesh in a selected direction, at a selected angle, and/or into a selected orientation. In an example, a device can include a coaxial catheter which further comprises an inner catheter and an outer catheter, wherein the inner and outer catheters are concentric and/or nested. In an example, a device can include a coaxial catheter which further comprises an inner catheter with a radially-asymmetric distal end and an outer catheter with a radially-symmetric distal end. In an example, a device can include a dual catheter, wherein the dual catheter further comprises first and second catheters which are parallel to each other, and wherein a first catheter has a smaller diameter than the second catheter.

In an example, a device can include a catheter that delivers embolic members and/or embolic material into an expandable mesh. In another example, a device can include a catheter that delivers embolic members and/or embolic material into an aneurysm sac through a opening in a proximal side (or surface) of expandable mesh. In an example, a device can include a catheter with a diagonally-cut end which a device operator can use to steer an expandable mesh in a selected direction, at a selected angle, or into a selected orientation.

In an example, a catheter can deliver an expandable mesh into an aneurysm sac via a pusher wire. In an example, a device can further comprise a conveyor belt and/or wire loop which is used to deliver an expandable mesh through a catheter to an aneurysm sac. In an example, a device can further comprise a liquid flow (e.g. saline flow) which is used to deliver embolic members and/or material through a catheter to an aneurysm sac. In an example, a device can further comprise a rotating helix (e.g. Archimedes screw) which is used to deliver an expandable mesh through a catheter to an aneurysm sac.

In an example, embolic members and/or material can comprise fiber strands. In another example, embolic members and/or material can comprise mesh ribbons. In an example, embolic members and/or material can comprise microsponges. In another example, embolic members and/or material can comprise string-of-pearls embolic strands. In an example, embolic components (e.g. beads, microspheres, microsponges, or hydrogels) in a string-of-pearls embolic strand can be connected by a single flexible longitudinal member (e.g. wire, suture, string, cord, filament, coil, or spring). In an example, distal embolic components (e.g. beads, microspheres, microsponges, or hydrogels) in a string-of-pearls embolic strand can be larger than proximal embolic components on the strand. In an example, distal embolic components (e.g. beads, microspheres, microsponges, or hydrogels) in a string-of-pearls embolic strand can be firmer and/or less elastic than proximal embolic components on the strand.

In an example, an embolic member can be a string-of-pearls embolic strand, wherein an string-of-pearls embolic strand comprises a longitudinal series (or sequence) of embolic components (e.g. beads, microspheres, microsponges, hydrogels, polyhedrons, or polymer masses) whose centers are connected (in a pairwise manner) by one or more flexible longitudinal members (e.g. wires, sutures, threads, strings, cords, or coils). In another example, distal embolic components (e.g. beads, microspheres, microsponges, hydrogels, polyhedrons, or polymer masses) in a string-of-pearls embolic strand can be closer together than proximal embolic components in the strand. In an example, the lengths of longitudinal strands connecting distal embolic components (e.g. beads, microspheres, microsponges, hydrogels, polyhedrons, or polymer masses) in a string-of-pearls embolic strand can be shorter than the lengths of longitudinal strands connecting proximal embolic components.

In an example, embolic components and/or material can be inserted into in aneurysm sac through a gap between an expandable mesh and an aneurysm wall. In an example, embolic components and/or material can be inserted into an aneurysm sac through one or more openings in a proximal side (or surface) of an expandable mesh. In an example, there can be a central opening in the proximal side (or surface) of an expandable mesh through which embolic members and/or material are inserted into an aneurysm sac, wherein the central opening is larger than openings or in the rest of the expandable mesh. In an example, there can be a plurality of openings in the proximal side (or surface) of an convex expandable mesh, wherein a device operator can select through which of these openings to insert embolic members and/or material into the convex expandable mesh.

In an example, a device can further comprise fluid-filled expandable components on an end portion of a catheter, wherein selectively filling one or more of the fluid-filled expandable components more than other expandable components steers an expandable mesh out of the catheter in a selected direction, at a selected angle, or into a selected orientation. In an example, a device can further comprise fluid-filled expandable components in or on an expandable mesh, wherein a device operator can steer the expandable mesh in a selected direction, at a selected angle, or into a selected orientation by selectively filling one or more of the fluid-filled expandable components more than other expandable components. In an example, a device can include a fluid conduit and/or channel on a catheter through which fluid is sent into a fluid-filled expandable component. In another example, a device operator can steer an expandable mesh by inserting different amounts of flowable substance into different expandable components in the mesh. In an example, an expandable component can be a balloon.

In an example, an expandable mesh can further comprise a plurality of inner chambers which can be selectively filled with embolic members or material, wherein selective filling of some of the chambers more than other chambers steers the expandable mesh in a selected direction, at a selected angle, and/or into a selected orientation. In an example, an expandable mesh can further comprise a plurality of inner chambers which can be selectively filled with embolic members or material, wherein different amounts of the embolic members and/or material are inserted into different chambers, thereby expanding the convex expandable mesh in a radially-asymmetric manner.

In an example, a device can comprise a plurality (e.g. longitudinal series) of wire or coil loops which are inserted into an aneurysm sac, wherein each loop is created separately and then attached to one or more other loops. In an example, a device can comprise a plurality (e.g. longitudinal series) of wire or coil loops which are inserted into an aneurysm sac, wherein the loops are created by looping and selectively binding branches of a single continuous wire or coil. In an example, a device can include a wire and/or coil loop which is inserted into an aneurysm sac, wherein the wire and/or coil loop has two branches (e.g. sides) which are parallel to each other as they are delivered through a catheter to the aneurysm sac.

In an example, a device can include a wire and/or coil loop which is inserted into an aneurysm sac, wherein the wire and/or coil loop has two branches (e.g. sides) which are parallel to each other as they are delivered through a catheter to the aneurysm sac, and wherein the end of the loop which is inserted into the aneurysm sac can be steered in a selected direction, at a selected angle, or into a selected orientation by selectively pushing one branch of the loop more than the other branch of the loop. In an example, a device can include a wire and/or coil loop which is inserted into an aneurysm sac, wherein the wire and/or coil loop has two branches (e.g. sides) which are parallel to each other as they are delivered through a catheter to the aneurysm sac, and wherein a device operator can steer the loop in a selected direction, at a selected angle, or into a selected orientation by selectively pushing one branch of the loop out farther from the end of a catheter than the other branch of the loop.

In an example, both branches (e.g. side) of a wire or coil loop can be delivered to an aneurysm sac through the same catheter. In another example, two branches (e.g. side) of a wire or coil loop can be delivered to an aneurysm sac through two different lumens of a multi-lumen catheter. In an example, adjacent wire or coil loops can be aligned along a central longitudinal axis of a catheter during delivery to an aneurysm sac, but intersect at acute angles after they exit the catheter. In an example, distal wire or coil loops in a longitudinal series of loops can be smaller than proximal wire or coil loops in the series. In an example, wire or coil loops can be inserted into an aneurysm sac. In an example, wire or coil loops which are inserted into an aneurysm sac can accumulate into a mass with a zigzag pattern, wherein angles between adjacent loops are less than 30 degrees.

In an example, a device can comprise a hydraulic actuator (e.g. hydraulic piston and/or microfluidic actuator). In an example, a device can comprise an annular array of actuators. In another example, a device can comprise electroconductive pathways (e.g. wires) along a catheter which provide power to one or more actuators which steer an expandable mesh. In an example, a device can comprise one or more actuators which change the shape of the end of a catheter. In an example, a device can comprise one or more actuators which steer an expandable mesh in a selected direction, at a selected angle relative to a catheter, and/or into a selected orientation relative to an aneurysm sac.

In an example, a device can comprise one or more actuators which a device operator uses to move (e.g. rotate, pivot, and/or tilt) a joint on a catheter which moves (e.g. pivots and/or tilts) an end portion of the catheter. In an example, a device can comprise one or more actuators which push or pull longitudinal members (e.g. wires or cords) which are attached to different branches of a coil loop. In an example, a device can comprise an actuator selected from the group consisting of: electric motor, electromagnetic actuator, hydraulic actuator, MEMS device, piezoelectric actuator, piston, pneumatic actuator, and solenoid.

In an example, a distal portion (e.g. the distal third or half) of an expandable mesh can be more porous than a proximal portion (e.g. the proximal third or half) of the expandable mesh. In an example, an expandable mesh can be a hexagonal (or honeycomb) mesh (e.g. with hexagonal pores or openings). In another example, an expandable mesh can be a polymer mesh. In an example, an expandable mesh can be made by 3D printing. In an example, an expandable mesh can be made by radially constraining a mesh tube at different locations. In an example, an expandable mesh can be made by radially constraining a mesh tube at different locations by welding, melting, and/or soldering In an example, an expandable mesh can be made by radially constraining a mesh tube at different locations by bands, rings, washers, or coils.

In an example, an expandable mesh can be a distally-opening concave (e.g. bowl, cup, or hemispherical, hemi-ellipsoidal, or funnel shaped) mesh. In an example, an expandable mesh can have a funnel or parabolic shape. In another example, an expandable mesh can have a hyperbolic or hourglass shape. In an example, an expandable mesh can have an oblate spheroidal shape. In an example, an expandable mesh can expand into a concave shape within an aneurysm sac. In an example, an expandable mesh can expand into a frustum shape within an aneurysm sac. In an example, an expandable mesh can expand into a half-torus shape within an aneurysm sac. In an example, an expandable mesh can expand into a pear shape within an aneurysm sac. In another example, an expandable mesh can expand into an apple shape within an aneurysm sac. In an example, an expandable mesh can expand into an hyperboloid shape within an aneurysm sac.

In an example, a device operator can cause and control radially-asymmetric expansion of a multi-layer expandable mesh by selectively filling a first gap between layers on a first (e.g. right or left) side of the mesh with embolic members and/or material more than a second gap between layers on a second (e.g. left or right) side of the mesh. In an example, a device operator can steer a multi-layer mesh in a selected direction, at a selected angle, or into a selected orientation by selectively filling a gap between layers on a first (e.g. right or left) side of the mesh with embolic members and/or material more than the gap between layers on a second (e.g. left or right) side of the mesh. In an example, an expandable mesh can comprise a proximal mesh and a distal mesh.

In an example, embolic members and/or material can be inserted between a proximal layer and a distal layer of an expandable mesh. In an example, accumulation of embolic members and/or material in a portion of an aneurysm sac which is distal to an expandable mesh can pressure, push, compel, and/or collapse a distal portion (e.g. distal half) of the expandable mesh into the concavity of a proximal portion (e.g. proximal half) of the expandable mesh. In an example, a convex (e.g. globular) expandable mesh can have a flexible annular section around its (central) circumference, wherein a distal portion (e.g. distal half) of the mesh is collapsed, compressed, and/or inverted around the annular section into a proximal portion (e.g. proximal half) of the mesh by pressure from accumulation of embolic members and/or material in a distal portion of the aneurysm sac.

In an example, an expandable mesh can be changed from a convex (e.g. globular) shape to a concave (e.g. bowl) shape by collapsing, compressing, and/or inverting a distal portion of the mesh into a concavity of a proximal portion of the mesh by pressure from accumulation of embolic members and/or material on the distal portion of the mesh. In another example, an expandable mesh can expand into a globular (e.g. spherical, ellipsoidal, or oblate spheroidal) shape in an aneurysm sac and then be collapsed, compressed, and/or inverted into a concave (e.g. bowl, cup, hemispherical, hemi-ellipsoidal) shape by pressure from the accumulation of embolic members and/or material in a distal portion of the aneurysm sac.

In an example, a device can comprise a convex distal mesh and a proximal mesh, wherein at least 80% of the (proximal-to-distal axis of) the distal mesh is nested within a concavity of a concave proximal mesh. In another example, a device can comprise a distal mesh and a proximal mesh, wherein at least 30% of the (proximal-to-distal axis of) the distal mesh is nested within a concavity of a proximal mesh. In another example, a device can comprise a distal mesh and a proximal mesh, wherein the proximal mesh is nested within the proximal half of the distal mesh. In an example, a device can comprise a distal mesh which is nested within a concavity of a proximal mesh, wherein the distal mesh has a lower mesh density than that of the proximal mesh. In an example, a device can comprise a distal mesh which is nested within a concavity of a proximal mesh, wherein the distal mesh is less rigid than the proximal mesh. In an example, a device can comprise a globular convex mesh which is nested within a concavity of a bowl-shaped concave mesh.

In an example, a device can comprise a distal flexible net or mesh which is nested within the concavity of a bowl-shaped concave mesh, wherein the distal flexible net or mesh is more flexible than the concave mesh. In another example, a device can comprise a distal flexible net or mesh which is nested within the concavity of a concave mesh, wherein embolic members and/or material is inserted through the concave mesh into the distal flexible net or mesh. In an example, a flexible net or mesh can be made by braiding or weaving metal wires or tubes.

In an example, a device can comprise a proximal-to-distal plurality (e.g. series, sequence, or stack) of expandable meshes which is inserted into an aneurysm sac, wherein central proximal-to-distal axes of the meshes are linearly aligned. In an example, a device can comprise a proximal-to-distal plurality (e.g. series, sequence, or stack) of convex expandable meshes which is inserted into an aneurysm sac. In an example, a device can comprise a proximal-to-distal plurality (e.g. series, sequence, or stack) of expandable meshes which is inserted sequentially (e.g. one at a time) into an aneurysm sac, wherein the expandable meshes are connected to each other by a flexible longitudinal member (e.g. wire, cord, string, suture, or coil).

In an example, a device can comprise a proximal-to-distal plurality (e.g. series, sequence, or stack) of expandable meshes which is inserted into an aneurysm sac, wherein the meshes are (centrally) connected to each other by a mechanism selected from the group consisting of: adhesive, braiding, central longitudinal member, clip, crimping, hook, melting, pinching, sewing, snap, soldering, tie wire, and weaving. In an example, a device can comprise a proximal-to-distal plurality (e.g. series, sequence, or stack) of expandable meshes are made by radially-constraining a continuous piece (e.g. from a tubular mesh) at multiple longitudinal locations. In an example, a device can comprise a proximal-to-distal plurality (e.g. series, sequence, or stack) of expandable meshes which is inserted into an aneurysm sac; wherein some of the meshes are nested in other meshes after deployment in an aneurysm sac.

In an example, a device can comprise a proximal-to-distal plurality (e.g. series, sequence, or stack) of expandable meshes which is inserted into an aneurysm sac, wherein a distal mesh in the series is more elastic than a proximal mesh in the series. In another example, a device can comprise a proximal-to-distal plurality (e.g. series, sequence, or stack) of expandable meshes which is inserted into an aneurysm sac, wherein the plurality includes a central or middle mesh between a proximal mesh and a distal mesh, and wherein the central or middle mesh is larger than the proximal mesh or the distal mesh.

In an example, a device can comprise a proximal-to-distal plurality (e.g. series, sequence, or stack) of expandable meshes which is inserted into an aneurysm sac, wherein the expandable meshes have oblate spheroidal shapes. In another example, a device can comprise a proximal-to-distal series or sequence of three or more expandable meshes which is inserted into an aneurysm sac. In an example, expandable meshes in a series can have: radially-compressed and longitudinally-lengthened first configurations when they are in a catheter being delivered to an aneurysm sac; and radially-expanded and longitudinally-shortened second configurations after they exit the catheter into the aneurysm sac.

In an example, distal expandable meshes in a longitudinal series (e.g. series, sequence, or stack) of expandable meshes can be larger (e.g. have larger diameters) than proximal expandable meshes in the series. In an example, distal expandable meshes in a longitudinal series (e.g. series, sequence, or stack) of expandable meshes can be more porous and/or have lower mesh densities than proximal expandable meshes in the series. In an example, distal expandable meshes in a longitudinal series (e.g. series, sequence, or stack) of expandable meshes can have different shapes than proximal expandable meshes in the series.

In an example, a device can comprise a longitudinal series (e.g. series, sequence, or stack) of expandable meshes which is inserted into an aneurysm sac, wherein there is a plurality of flexible longitudinal members (e.g. wires, sutures, cords, strings, or coils) connected to the expandable meshes at different locations, and wherein pulling, pushing, or rotating a subset of the longitudinal members steers the longitudinal series in a selected direction, at a selected angle, or into a selected orientation.

In an example, an annular section around a central circumference of an expandable mesh can be more elastic than the rest of the mesh. In another example, an annular section around the (central) circumference of an expandable mesh can comprise between 2% and 10% of the (surface area of the) expandable mesh. In an example, an expandable mesh can comprise an annular section around a (central) conference of the mesh, wherein the annular section is more flexible, more elastic, and/or thinner than the rest of the mesh] so that a distal portion (e.g. distal half) of the mesh can collapse and/or invert into a proximal portion (e.g. proximal half) of the mesh when pressure is applied to the distal portion by the accumulation of embolic members and/or embolic material in an aneurysm sac.

In an example, a device can comprise a band or ring around a (central) circumference of a net or mesh, wherein the band or ring is at least 25% greater than the diameter of an aneurysm neck after the band or ring has expanded within an aneurysm sac. In an example, a device can comprise a band or ring which spans between 5% and 20% of the surface area of a net or mesh. In an example, a device can further comprise a band or ring around the outside of a (central) circumference of a net or mesh, wherein the band or ring is less flexible, less elastic, less compressible, and/or more resilient than the net or mesh. In an example, an expandable mesh can include a band or ring which radially-constrains the mesh.

In an example, an expandable mesh can include a band or ring with multiple sections, wherein selective detachment of one or more of the sections enables a device operator to control radially-asymmetric expansion of the mesh. In an example, an expandable mesh can include a selectively-detachable band or ring which radially-constrains the mesh. In an example, an expandable mesh can have multiple coaxial lobes and/or undulations. In an example, an expandable mesh can have multiple lobes or undulations, wherein a distal lobe or undulation is larger than a proximal lobe or undulation. In another example, an expandable mesh can have multiple lobes or undulations, wherein a proximal lobe or undulation is at least 20% larger than a distal lobe or undulation. In an example, an expandable mesh can have multiple radial lobes and/or undulations. In an example, an expandable mesh can have sinusoidal radial lobes or undulations.

In an example, a device can include a plurality of detachable connections which a plurality of radial constraints to the circumference of an expandable mesh, wherein selective detachment of a subset of one or more of the detachable connections causes asymmetric radial expansion of the expandable mesh. In an example, a device can include a radial constraint on an expandable mesh wherein detachment a connection on the radial constraint releases a portion of the expandable mesh from radial constraint. In an example, a device can include one or more radial constraints on an expandable mesh, wherein selective detachment of one or more sections of the radial constraints causes the mesh to expand in a radially-asymmetric manner.

In an example, a device can include one or more radial constraints on an expandable mesh, a device operator can selectively detach one or more sections of the radial constraints steers the mesh in a selected direction, at a selected angle, or into a selected orientation. In an example, a device can include one or more selectively-detachable radial constraints on an expandable mesh, wherein a device operator can selectively detach one or more of the radial constraints to cause the mesh to expand in a radially-asymmetric manner. In another example, a device can include one or more selectively-detachable circumferential constraints on an expandable mesh, wherein a device operator can selectively detach one or more of the circumferential constraints to cause the mesh to expand in a longitudinally-asymmetric manner.

In an example, a device can include one or more selectively-detachable radial constraints on an expandable mesh, wherein a device operator can selectively detach one or more of the radial constraints to steer the mesh in a selected direction, at a selected angle, or into a selected orientation. In an example, a device can include one or more selectively-detachable circumferential constraints on an expandable mesh, wherein a device operator can selectively detach one or more of the circumferential constraints to steer the mesh in a selected direction, at a selected angle, or into a selected orientation. In an example, a device operator can selectively detach one or more (sections of) radial or longitudinal constraints on an expandable mesh in order to cause the mesh to expand in a radially-asymmetric manner.

In an example, a device can comprise a circumferential constraint (e.g. ring or band) with a plurality of detachable sections around a distal circumference of a concave expandable mesh. In another example, a device can comprise a circumferential constraint (e.g. ring or band) with a plurality of detachable sections around a distal circumference of a concave expandable mesh, wherein a device operator can cause the expandable mesh to expand in a radially-asymmetric manner by selectively detaching a subset of the detachable sections. In an example, a device can comprise a detachable circumferential constraint (e.g. ring or band) around a central circumference of an expandable mesh. In an example, a device can comprise one or more detachable circumferential constraints (e.g. rings or bands) around one or more circumferences of an expandable mesh. In an example, a detachable constraint can be detached by the transmission of electrical energy. In another example, a device can include a heating element which melts a detachable connection on an expandable mesh. In an example, a device can include an actuator which cuts (or otherwise detaches) a detachable connection on an expandable mesh.

In an example, a device operator can control the radially-asymmetric expansion of an expandable mesh in order to better occlude an irregularly-shape aneurysm sac. In another example, a device operator can control the radially-asymmetric expansion of an expandable mesh in order to steer the mesh in a selected direction, at a selected angle, or into a selected orientation within an aneurysm sac. In an example, an expandable mesh can be expanded in a radially-asymmetric manner.

In an example, a device which enables expansion of an expandable mesh into a radially-asymmetric shape can enable a device operator to fill an irregularly-shaped aneurysm sac more thoroughly and completely than a device with an expandable mesh with a preset (e.g. spherical or barrel shaped) mesh. In an example, a device which enables steering an expandable mesh in a selected direction, at a selected angle, and/or into a selection orientation can enable a device operator to fill an aneurysm sac more thoroughly, completely, and/or quickly than a device which does not enable steering an expandable mesh, especially if the aneurysm is irregularly shaped. In an example, a device can comprise two flexible longitudinal members (e.g. wires) which are attached to opposite sides of an expandable mesh.

In an example, a device can further comprise one or more actuators which push, pull, or rotate flexible longitudinal members to control radially-asymmetric expansion of an expandable mesh. In an example, a device can include a plurality of flexible longitudinal members (e.g. wires) which span the length of a catheter and are connected at different locations to an expandable mesh, wherein the expandable mesh is steered in a selected direction, at a selected angle, or into a selected orientation when one or more of the flexible longitudinal members are pulled, pushed, and/or rotated.

In an example, a device can include a plurality of flexible longitudinal members (e.g. wires) which are connected at different locations to an expandable mesh, wherein the expandable mesh expands in a selected radially-asymmetric manner when a subset of the flexible longitudinal members are selectively pulled, pushed, and/or rotated by the device operator. In an example, a device can include one or more flexible longitudinal members (e.g. wires) which are connected to an expandable mesh, wherein the expandable mesh is steered in a selected direction, at a selected angle, or into a selected orientation when one or more of the flexible longitudinal members are pulled, pushed, and/or rotated.

In an example, a device can include one or more flexible longitudinal members (e.g. wires) which span the length of a catheter and are connected to an expandable mesh, wherein the expandable mesh is steered in a selected direction, at a selected angle, or into a selected orientation when one or more of the flexible longitudinal members are selectively pulled, pushed, and/or rotated by a device operator. In another example, a device can include one or more flexible longitudinal members (e.g. wires) which span the length of a catheter and are connected to an expandable mesh, wherein the expandable mesh expands in a selected radially-asymmetric manner when one or more of the flexible longitudinal members are pulled, pushed, and/or rotated.

In an example, a device operator can control asymmetric expansion of an expandable mesh by selectively transmitting electricity to one or more piezoelectric members (e.g. piezoelectric cords or strands) which are connected to different locations on the mesh. In another example, a device operator can selectively steer an expandable mesh left or right, or up or down, by differentially pulling, pushing, or rotating flexible longitudinal members. In an example, a flexible longitudinal member can be a suture, thread, or string. In another example, a flexible longitudinal member can be piezoelectric. In an example, a piezoelectric flexible longitudinal member can be shrunk or elongated by transmission of electrical energy.

In an example, a device can comprise an expandable mesh with multiple openings (e.g. including a central opening and multiple non-central openings), wherein each opening has a valve, wherein a device operator can select which of the openings to use to insert expandable members and/or material through a selected opening into an aneurysm sac, and wherein the device operator selectively opens the valve for the selected opening. In an example, a device can comprise an expandable mesh with multiple potential opening locations (e.g. a central location and multiple non-central locations), wherein a device operator selects which of the potential opening locations to open to insert expandable members and/or material into an aneurysm sac, and wherein the device operator opens a selected opening location by cutting.

In an example, an opening in a multi-layer mesh can be through all of the layers of the mesh. In an example, embolic members and/or material can be inserted into an aneurysm sac through a central opening of a torus shaped expandable mesh. In another example, there can be a central opening in an expandable mesh through which embolic members and/or material are inserted into an aneurysm sac. In an example, there can be a plurality of openings on an expandable mesh, wherein there is a one-way valve on each of the openings. In another example, there can be an opening in a convex expandable mesh through which embolic members and/or material are inserted into the interior of the convex expandable mesh.

In an example, there can be an opening in an expandable mesh through which embolic members and/or material are inserted into the interior of the expandable mesh. In an example, there can be one or more openings in an expandable mesh through which embolic members and/or material are inserted into an aneurysm sac, wherein the opening is at least 50% larger than pores between mesh wires or strands elsewhere on the mesh. In an example, a device operator can remotely control the operation of a valve in an opening through an expandable mesh using an adhesive. In an example, a device operator can remotely control the operation of a valve in an opening through an expandable mesh using a cap. In another example, a device operator can remotely control the operation of a valve in an opening through an expandable mesh using a clip.

In an example, a device operator can remotely control the operation of a valve in an opening through an expandable mesh using a fusible member. In another example, a device operator can remotely control the operation of a valve in an opening through an expandable mesh using a loop. In an example, a device operator can remotely control the operation of a valve in an opening through an expandable mesh using a plug. In an example, a device operator can remotely control the operation of a valve in an opening through an expandable mesh using a pull wire.

In an example, a device operator can remotely control the operation of a valve in an opening through an expandable mesh using a snap. In an example, a subset of openings can be remotely opened by a device operator via targeted transmission of light energy. In an example, there can be a valve in an opening through an expandable mesh through which embolic members and/or material can be inserted into an aneurysm sac. In an example, there can be a valve in an opening through an expandable mesh through which embolic members and/or material can be inserted into an aneurysm sac, wherein a device operator can control the operation of the valve remotely.

In an example, an expandable mesh can be made by 3D printing a polymer ink. In an example, an expandable mesh can be made from an elastomeric polymer. In another example, an expandable mesh can be formed from a combination of metal wires (or tubes) and polymer strands, filaments, and/or threads. In an example, an expandable mesh can be made by laser-cutting holes in a globular polymer structure (e.g. a balloon). In an example, a flexible net or mesh can be made by braiding or weaving polymer strands, filaments, threads, or yarns. In an example, a flexible net or mesh can be made from one or more polymers. In an example, a flexible net or mesh can be made by braiding and/or weaving wires and polymer strands, filaments, and/or threads together. In an example, one or more portions of a flexible net or mesh can be made from a radiogenic material. In another example, one or more portions of an expandable mesh can be made from a radiogenic material. In an example, an expandable mesh can be made with a polymer which has been doped or impregnated with radio-opaque and/or radiopacifying material.

In an example, a device can include a catheter with a diagonal end which causes an expandable mesh to expand in a radially-asymmetric manner. In an example, a device can include a catheter with a radially-asymmetric end which steers an expandable mesh in a selected direction, at a selected angle, or into a selected orientation. In an example, a device operator can use a catheter with a diagonally-cut end to steer an expandable mesh in a selected direction, at a selected angle, or into a selected orientation. In an example, a device operator can use a catheter with a radially-asymmetric end to steer an expandable mesh in a selected direction, at a selected angle, or into a selected orientation. In another example, an end portion of a catheter can be curved at an acute angle like a piece of plumbing pipe. In another example, the angle of the end of a dual catheter can be changed by longitudinally sliding an inner catheter relative to an outer catheter, or vice versa.

In an example, a device can further comprise one or more electromagnetic actuators (e.g. electric motors) which move (e.g. rotated, pivot, and/or tilt) an end portion of a catheter to steer an expandable mesh in a selected direction, at a selected angle, and/or with a selected orientation. In an example, a movable end portion of a catheter can direct (e.g. steer, compel, or push) an expandable mesh out of the catheter in a selected direction, at a selected angle, and/or with a selected orientation. In an example, an end portion of a catheter can be connected to the rest of the catheter by a joint, wherein the end portion is moved (e.g. rotated, pivoted, and/or tilted) by the joint in order to steer an expandable mesh in a selection direction, at a selected angle, or with a selected orientation.

In an example, an end portion of a catheter can be moved (e.g. rotated, pivoted, and/or tilted) in order to steer an expandable mesh in a selection direction, at a selected angle, or with a selected orientation as the mesh exits the catheter. In another example, a device can include a movable (e.g. rotatable, pivoting, and/or tilting) joint between a catheter and an expandable mesh, wherein the movable joint enables a device operator to steer the expandable mesh in a selected direction, at a selected angle, or into a selected orientation. In another example, a device operator can rotate and/or pivot a joint by rotating a wire connect to the joint. In an example, a device operator can use one or more electromagnetic actuators (e.g. micromotors) to move a joint between a catheter and an expandable mesh.

In an example, a device operator can use one or more hydraulic actuators (e.g. micropistons) to move a joint between an end portion of a catheter and the rest of the catheter. In an example, a joint between a catheter and an expandable mesh can be moved by one or more hydraulic actuators (e.g. micropistons). In an example, a joint between an end portion of a catheter and the rest of the catheter can be moved by one or more electromagnetic actuators (e.g. micromotors). In another example, a movable joint can be a rotating joint. In an example, a device operator can change a direction, angle, and/or orientation of an expandable mesh by selectively moving (e.g. pivoting and/or rotating) a movable joint between the mesh and a catheter.

In an example, a device can include a coaxial catheter wherein an outer catheter can slide distally relative to an inner catheter and/or the inner catheter can slide proximally relative to the outer catheter In an example, a device can include a coaxial catheter wherein the dual catheter further comprises first and second catheters which are coaxial, concentric, and/or nested relative to each other. In an example, a device can include a coaxial catheter which further comprises an inner catheter and an outer catheter, wherein one or both of the inner and outer catheters slides relative to the other. In an example, a device can include a dual and/or coaxial catheter.

In an example, a device can include first and second catheters which are parallel to each other, wherein a first branch of a coil loop is inserted through the first catheter and a second branch of the coil loop is inserted through the second catheter. In an example, a device can include a catheter that delivers an expandable mesh into an aneurysm sac. In an example, a device can include a catheter that delivers embolic members and/or embolic material into an convex expandable mesh. In another example, a device can include a catheter that delivers embolic members and/or embolic material into an aneurysm sac through a gap between an expandable mesh and a wall of the aneurysm sac. In an example, a device can include a catheter with a movable end portion which a device operator can use to steer an expandable mesh in a selected direction, at a selected angle, or into a selected orientation.

In an example, a catheter can deliver embolic members and/or material into an aneurysm sac. In an example, a device can further comprise a conveyor belt and/or wire loop which is used to deliver embolic members and/or material through a catheter to an aneurysm sac. In an example, a device can further comprise a pusher wire or plunger which is to deliver an expandable mesh through a catheter to an aneurysm sac. In an example, a device can further comprise a rotating helix (e.g. Archimedes screw) which is used to deliver embolic members and/or material through a catheter to an aneurysm sac. In an example, embolic members and/or material can comprise beads. In an example, embolic members and/or material can comprise hydrogels. In an example, embolic members and/or material can comprise microbeads.

In an example, embolic members and/or material can comprise polymer coils. In an example, a string-of-pearls embolic strand can comprise a plurality (e.g. a longitudinal series) of embolic components (e.g. beads, microspheres, microsponges, or hydrogels) which are connected along a flexible longitudinal member (e.g. wire, suture, string, cord, filament, coil, or spring). In another example, embolic components (e.g. beads, microspheres, microsponges, or hydrogels) in a string-of-pearls embolic strand can be connected by a two or more flexible longitudinal members (e.g. wires, sutures, strings, cords, filaments, coils, or springs). In an example, distal embolic components (e.g. beads, microspheres, microsponges, or hydrogels) in a string-of-pearls embolic strand can be closer together than proximal embolic components on the strand.

In an example, distal embolic components (e.g. beads, microspheres, microsponges, or hydrogels) in a string-of-pearls embolic strand can be made from a different material than proximal embolic components on the strand. In an example, an embolic member can be a string-of-pearls embolic strand, wherein an string-of-pearls embolic strand comprises a longitudinal series (or sequence) of embolic components connected (in a pairwise manner) by a flexible longitudinal member. In an example, distal embolic components (e.g. beads, microspheres, microsponges, hydrogels, polyhedrons, or polymer masses) in a string-of-pearls embolic strand can be softer, more compressible, have lower durometer levels than proximal embolic components in the strand. In an example, distal embolic components (e.g. beads, microspheres, microsponges, hydrogels, polyhedrons, or polymer masses) in a string-of-pearls embolic strand can be made from different material than the material used to make proximal embolic components in the strand.

In an example, embolic components and/or material can be inserted into in aneurysm sac through a gap between the perimeter and/or circumference of an expandable mesh and an aneurysm wall. In another example, embolic components and/or material can be inserted into an aneurysm sac through a selected openings in a proximal side (or surface) of an expandable mesh. In another example, there can be a plurality of non-central openings in the proximal side (or surface) of an expandable mesh, wherein a device operator can select through which of these openings to insert embolic members and/or material into an aneurysm sac.

In an example, a device can further comprise fluid-filled expandable components on an end portion of a catheter, wherein a device operator can steer an expandable mesh out of the catheter in a selected direction, at a selected angle, or into a selected orientation by selectively filling one or more of the fluid-filled expandable components more than other expandable components. In an example, a device can further comprise fluid-filled expandable components in or on an expandable mesh, wherein selectively filling one or more of the fluid-filled expandable components more than other expandable components causes asymmetric expansion of the expandable mesh. In an example, a device can include a plurality of a fluid conduits and/or channels on a catheter through which fluid is sent into a plurality of fluid-filled expandable components.

In an example, a multi-layer expandable mesh can further comprise a plurality of fluid-filled components between layers of the mesh, wherein selectively filling a first component to a greater extent than filling a second component steers the expandable mesh in a selected direction, at a selected angle, and/or into a selected orientation. In an example, an expandable component can be filled with saline solution. In another example, a device operator can fill different chambers in an expandable mesh with different amounts of embolic members or material in order to steer the expandable mesh in a selected direction, at a selected angle, and/or into a selected orientation. In an example, an expandable mesh can further comprise a plurality of inner chambers which can be selectively filled with embolic members or material, wherein selective filling of some of the chambers more than other chambers causes the expandable mesh to expand in a radially-asymmetric manner.

In an example, an expandable mesh can further comprise a plurality of inner chambers which can be selectively filled with embolic members or material, wherein different amounts of the embolic members and/or material are inserted into different chambers, thereby steering the mesh in a selected direction, at a selected angle, or into a selected orientation. In an example, a device can comprise a plurality (e.g. longitudinal series) of wire or coil loops which are inserted into an aneurysm sac. In an example, a device can comprise a plurality (e.g. longitudinal series) of wire or coil loops which are inserted into an aneurysm sac, wherein loops are created separately and then attached to each other.

In an example, a device can comprise a plurality (e.g. longitudinal series) of wire or coil loops which are inserted into an aneurysm sac, wherein the loops are created by looping and binding branches of a single continuous wire or coil together at a series of longitudinal locations. In an example, a device can include a wire and/or coil loop which is inserted into an aneurysm sac, wherein the wire and/or coil loop has two branches (e.g. sides) which delivered through a dual catheter to the aneurysm sac, and wherein there is one branch in each of two catheters in the dual catheter.

In an example, a device can include a wire and/or coil loop which is inserted into an aneurysm sac, wherein the wire and/or coil loop has two branches (e.g. sides) which are parallel to each other as they are delivered through a catheter to the aneurysm sac, and wherein the end of the loop which is inserted into the aneurysm sac can be steered in a selected direction, at a selected angle, or into a selected orientation by selectively pushing more of one branch of the loop into the aneurysm sac than the other branch of the loop. In another example, a device operator can push or pull one or both branches of a wire or coil loop from a location outside a person's body. In an example, rotation of a first branch (e.g. side) of a wire or coil loop relative to a second branch (e.g. side) of the loop can cause the branches to intertwine (e.g. wind around) each other and form an occlusive mass within an aneurysm sac.

In an example, adjacent wire or coil loops can be colinear during delivery through a catheter, but their best-fitting virtual planes can intersect at acute angles after they exit the catheter. In an example, the orientations of sequential loops in a longitudinal series of wire or coil loops can alternate sequentially (e.g. from side to side) after they deployed in an aneurysm sac. In an example, wire or coil loops which are inserted into an aneurysm sac can accumulate into a beehive-shaped mass.

In an example, a device can comprise a piezoelectric actuator (e.g. piezoelectric pull cord). In an example, a device can comprise an electromagnetic actuator (e.g. electric motor, solenoid, or MEMS device). In an example, a device can comprise electroconductive pathways (e.g. wires) along a catheter which transmit power and/or commands to one or more actuators at the end of the catheter. In an example, a device can comprise one or more actuators which move (e.g. rotate, pivot, and/or tilt) the end of a catheter. In another example, a device can comprise one or more actuators which a device operator uses to steer an expandable mesh in a selected direction, at a selected angle, or into a selected orientation.

In an example, a device can comprise one or more actuators which a device operator uses to selectively open or close one or more openings on a proximal side of an expandable mesh, wherein embolic members and/or material are inserted through the one or more openings into the aneurysm sac. In an example, a device can comprise one or more actuators which are used by a device operator to steer an expandable mesh to the right, to the left, up, or down. In an example, a distal portion (e.g. the distal third or half) of an expandable mesh can be more elastic and/or stretchable than a proximal portion (e.g. the proximal third or half) of the expandable mesh. In an example, a distal portion (e.g. the distal third or half) of a net or mesh can be more porous than a proximal portion (e.g. the proximal third or half) of the net or mesh.

In an example, an expandable mesh can be a lattice. In an example, an expandable mesh can be a stent. In an example, an expandable mesh can be made by braiding and/or weaving wires. In an example, an expandable mesh can be made by radially constraining a mesh tube at different locations by wires, twist ties, strings, or sutures. In an example, an expandable mesh can self-expand within an aneurysm sac after it exits a catheter. In an example, a compound expandable mesh can comprise a concave (e.g. bowl, cup, or hemispherical, hemi-ellipsoidal, or funnel shaped) mesh within (a proximal half of) a distally-opening convex (e.g. globular, spherical, ellipsoidal, or oblate spherical) mesh.

In an example, an expandable mesh can have a bowl or cup shape. In an example, an expandable mesh can have a half-torus shape (e.g. like the lower half of a cut bagel). In an example, an expandable mesh can have a radially-asymmetric shape. In an example, there can be a low-porosity film or fabric spanning a central opening of a toroidal mesh. In an example, an expandable mesh can expand into a convex shape within an aneurysm sac. In an example, an expandable mesh can expand into a funnel shape within an aneurysm sac. In another example, an expandable mesh can expand into a hemispherical shape within an aneurysm sac. In an example, an expandable mesh can expand into a spherical shape within an aneurysm sac.

In an example, an expandable mesh can expand into an ellipsoidal shape within an aneurysm sac. In an example, an expandable mesh can expand into an oblate spheroidal shape within an aneurysm sac. In another example, a concave expandable mesh can be a dual-layer mesh. In an example, a device operator can cause and control radially-asymmetric expansion of an multi-layer expandable mesh by selectively filling a gap between layers on a first (e.g. right or left) side of the mesh with embolic members and/or material more than the gap between layers on a second (e.g. left or right) side of the mesh. In an example, a two-layer concave mesh can be formed by collapsing, compressing, and/or inverting a single-layer convex mesh. In an example, an expandable mesh can have two layers.

In an example, a convex (e.g. globular, expandable mesh can have a flexible annular section around its (central) circumference, wherein a distal portion (e.g. distal half) of the mesh is collapsed, compressed, and/or inverted around the annular section into a proximal portion (e.g. proximal half) of the mesh. In an example, an expandable mesh can be changed from a convex (e.g. globular) shape to a concave (e.g. bowl) shape by collapsing, compressing, and/or inverting a distal portion of the mesh into a concavity of a proximal portion of the mesh. In an example, an expandable mesh can expand into a globular (e.g. spherical, ellipsoidal, or oblate spheroidal) shape in an aneurysm sac and then be collapsed, compressed, and/or inverted into a concave (e.g. bowl, cup, hemispherical, hemi-ellipsoidal) shape.

In an example, an expandable mesh can expand into a single-layer globular (e.g. spherical, ellipsoidal, or oblate spheroidal) shape in an aneurysm sac and then be collapsed, compressed, and/or inverted into a two-layer concave (e.g. bowl, cup, hemispherical, hemi-ellipsoidal) shape. In another example, a device can comprise a convex distal mesh and a proximal mesh, wherein at least 30% of the (proximal-to-distal axis of) the distal mesh is nested within a concavity of a concave proximal mesh. In another example, a device can comprise a convex distal mesh which is nested within a concavity of a proximal mesh. In an example, a device can comprise a distal mesh and a proximal mesh, wherein at least 45% of the (proximal-to-distal axis of) the distal mesh is nested within a concavity of a proximal mesh.

In an example, a device can comprise a distal mesh which is nested within a concavity of a proximal mesh. In an example, a device can comprise a distal mesh which is nested within a concavity of a proximal mesh, wherein the distal mesh his more elastic and/or stretchable than the proximal mesh. In an example, a device can comprise a distal mesh which is nested within a concavity of a proximal mesh, wherein the distal mesh is made with a higher proportion of polymer material than the proximal mesh. In an example, a device can comprise a multi-layer mesh, wherein the multi-layer mesh comprises a convex inner mesh which is inside a convex outer mesh.

In an example, a device can comprise a distal flexible net or mesh which is nested within the concavity of a concave mesh, wherein the distal flexible net or mesh is more porous than the concave mesh. In another example, a flexible net or mesh can be a honeycomb mesh (e.g. with hexagonal pores or openings). In another example, a flexible net or mesh can be made by braiding or weaving polymer strands, fibers, or threads. In an example, a device can comprise a proximal-to-distal plurality (e.g. series, sequence, or stack) of expandable meshes which is inserted into an aneurysm sac. In an example, a device can comprise a proximal-to-distal plurality (e.g. series, sequence, or stack) of expandable meshes which is inserted into an aneurysm sac, wherein the meshes are centrally-connected by a flexible longitudinal member (e.g. wire, cord, suture, string, or coil).

In an example, a device can comprise a proximal-to-distal plurality (e.g. series, sequence, or stack) of concave expandable meshes which is inserted into an aneurysm sac. In an example, a device can comprise a proximal-to-distal plurality (e.g. series, sequence, or stack) of expandable meshes which is inserted sequentially (e.g. one at a time) into an aneurysm sac, wherein the expandable meshes are connected to each other by a flexible longitudinal member (e.g. wire, cord, string, suture, or coil), and wherein one or more distances between the expandable meshes are adjusted after their insertion into the aneurysm sac by pulling, pushing, or rotating the flexible longitudinal member. In an example, a device can comprise a proximal-to-distal plurality (e.g. series, sequence, or stack) of expandable meshes which is inserted into an aneurysm sac, wherein the meshes are made separately and then connected to each other.

In an example, a device can comprise a proximal-to-distal plurality (e.g. series, sequence, or stack) of expandable mesh lobes which are made by radially-constraining a continuous piece (e.g. from a tubular mesh) at multiple longitudinal locations. In another example, a device can comprise a proximal-to-distal plurality (e.g. series, sequence, or stack) of expandable meshes which is inserted into an aneurysm sac, wherein a distal mesh in the series is smaller than a proximal mesh in the series. In an example, a device can comprise a proximal-to-distal plurality (e.g. series, sequence, or stack) of expandable meshes which is inserted into an aneurysm sac, wherein a distal mesh in the series has a lower durometer level and/or Shore value than a proximal mesh in the series.

In an example, a device can comprise a proximal-to-distal plurality (e.g. series, sequence, or stack) of expandable meshes which is inserted into an aneurysm sac, wherein the plurality includes a central or middle mesh between a proximal mesh and a distal mesh, wherein the central or middle mesh is convex, and wherein the proximal mesh and the distal mesh are each concave. In an example, a device can comprise a proximal-to-distal plurality (e.g. series, sequence, or stack) of expandable meshes which is inserted into an aneurysm sac, wherein the expandable meshes have ellipsoidal shapes. In an example, an expandable mesh can have: a radially-compressed and longitudinally-lengthened first configuration when it is in a catheter being delivered to an aneurysm sac; and a radially-expanded and longitudinally-shortened second configuration after it exits the catheter into the aneurysm sac.

In an example, plurality of separately-formed expandable meshes can be connected together by a mechanism selected from the group consisting of: adhesion, soldering, welding, crimping, pinching, tying, braiding, weaving, snapping, clipping, and hooking. In another example, distal expandable meshes in a longitudinal series (e.g. series, sequence, or stack) of expandable meshes can be closer together than proximal expandable meshes in the series. In an example, distal expandable meshes in a longitudinal series (e.g. series, sequence, or stack) of expandable meshes can be smaller (e.g. have smaller diameters) than proximal expandable meshes in the series. In an example, distal expandable meshes in a longitudinal series (e.g. series, sequence, or stack) of expandable meshes can have lower durometer levels and/or Shore values than proximal expandable meshes in the series.

In an example, distal expandable meshes in a longitudinal series (e.g. series, sequence, or stack) of expandable meshes can have lower mesh densities than proximal expandable meshes in the series. In an example, a device can comprise a longitudinal series (e.g. series, sequence, or stack) of expandable meshes which is inserted into an aneurysm sac, wherein there are one or more flexible longitudinal members (e.g. wires, sutures, cords, strings, or coils) connected to the expandable meshes, and wherein pulling, pushing, or rotating a subset of the longitudinal members steers the longitudinal series in a selected direction, at a selected angle, or into a selected orientation. In an example, a device can comprise a longitudinal series (e.g. series, sequence, or stack) of expandable meshes which is inserted into an aneurysm sac, wherein there is a plurality of flexible longitudinal members (e.g. wires, sutures, cords, strings, or coils) connected to the expandable meshes at non-central locations, and wherein pulling, pushing, or rotating a subset of the longitudinal members steers the longitudinal series in a selected direction, at a selected angle, or into a selected orientation.

In an example, the braid or weave of wires, tubes, or fibers comprising an annular section of an expandable mesh can be more sparse, less dense, more open, and/or more loose than that of the rest of the mesh In another example, a flexible annular section around a circumference of an expandable mesh can span the central circumference of the mesh. In an example, an annular section around a central circumference of an expandable mesh can be more flexible than the rest of the mesh. In another example, an annular section around the (central) circumference of an expandable mesh can comprise between 5% and 20% of the (surface area of the) expandable mesh. In an example, wires, tubes, and/or strands used to form an annular section around a central circumference of an expandable mesh can be more flexible, more elastic, thinner, smaller, and/or woven less densely than those in rest of the mesh. In an example, a band or ring around an expandable mesh can be around the central circumference of the mesh.

In an example, a device can comprise a band or ring around a (central) circumference of a net or mesh, wherein the band or ring is at least 80% of the maximum diameter of an aneurysm sac after the band or ring has expanded within the aneurysm sac. In another example, a device can further comprise a band or ring around a (central) circumference of a net or mesh. In an example, a device can further comprise a band or ring around the inside of a (central) circumference of a net or mesh, wherein the band or ring is less flexible, less elastic, less compressible, and/or more resilient than the net or mesh. In another example, an expandable mesh can include a band or ring which constrains radial expansion of the mesh. In an example, an expandable mesh can include a detachable band or ring which radially-constrains the mesh.

In an example, an expandable mesh can have multiple lobes and/or undulations. In an example, an expandable mesh can have multiple lobes or undulations, wherein a proximal lobe or undulation is larger than a distal lobe or undulation. In an example, an expandable mesh can have multiple lobes or undulations, wherein a proximal lobe or undulation is more dense and/or less porous than a distal lobe or undulation. In an example, an expandable mesh can have sinusoidal lobes or undulations. In an example, the perimeter of an expandable mesh can have multiple lobes and/or undulations. In an example, a device can include a plurality of detachable connections on one or more proximal-to-distal constraints, wherein selective detachment of a subset of one or more of the detachable connections causes asymmetric proximal-to-distal expansion of an expandable mesh.

In an example, a device can include a radial array (e.g. spoke array) of detachable wires. In another example, a device can include at least six detachable radial constraints on an expandable mesh. In an example, a device can include one or more radial constraints on an expandable mesh, a device operator can selectively detach one or more sections of the radial constraints to cause the mesh to expand in a radially-asymmetric manner. In an example, a device can include one or more selectively-detachable constraints on an expandable mesh, wherein selective detachment of one or more of the constraints causes the mesh to expand in an asymmetric manner.

In an example, a device can include one or more selectively-detachable longitudinal constraints on an expandable mesh, wherein a device operator can selectively detach one or more of the longitudinal constraints to cause the mesh to expand in a longitudinally-asymmetric manner. In another example, a device can include one or more selectively-detachable constraints on an expandable mesh, wherein selective detachment of one or more of the constraints steers the mesh in a selected direction, at a selected angle, or into a selected orientation.

In an example, a device can include one or more selectively-detachable longitudinal constraints on an expandable mesh, wherein a device operator can selectively detach one or more of the longitudinal constraints to steer the mesh in a selected direction, at a selected angle, or into a selected orientation. In another example, a device operator can selectively detach one or more (sections of) radial or longitudinal constraints on an expandable mesh in order to steer the mesh in a selected direction, at a selected angle, or into a selected orientation. In an example, a device operator can selectively detach one or more (sections of) radial or longitudinal constraints on an expandable mesh by transmitting electricity to them in order to cause the mesh to expand in an asymmetric manner.

In an example, a device can comprise a circumferential constraint (e.g. ring or band) with a plurality of detachable sections around a distal circumference of a concave expandable mesh, wherein a device operator can steer the expandable mesh in a selected direction, at a selected angle, or with a selected orientation by selectively detaching a subset of the detachable sections. In an example, a device can comprise a detachable circumferential constraint (e.g. ring or band) around a circumference of an expandable mesh. In an example, a device can comprise a detachable circumferential constraint (e.g. ring or band) around a distal circumference of a concave expandable mesh.

In an example, a detachable constraint can be detached by the transmission of electrical energy to a connection location. In another example, a device can include a light emitter which melts a detachable connection on an expandable mesh. In an example, a device can include an actuator which cuts (or otherwise detaches) a detachable connection on a radial constraint on an expandable mesh. In an example, a device operator can control the expansion of an expandable mesh so that a first side of the mesh expands more rapidly and/or to a greater extent than a second side of the mesh. In an example, a device operator can control the radially-asymmetric expansion of an expandable mesh in order to better occlude an aneurysm which is accessed from a parent vessel at an acute angle.

In an example, a device operator can steer an expandable mesh in a selected direction, at a selected angle, or into a selected orientation. In an example, an expandable mesh can be expanded in a radially-asymmetric manner within an aneurysm. In another example, a device which enables control over asymmetric expansion of an expandable mesh in an aneurysm sac can enable a device operator to fill an irregularly-shaped aneurysm sac more thoroughly, completely, and/or quickly than a device which does not enable asymmetric expansion an expandable mesh. In an example, a device which enables expansion of an expandable mesh into a radially-asymmetric shape can enable a device operator to fill an aneurysm sac more thoroughly and completely than a device with an expandable mesh with a preset (e.g. spherical or barrel shaped) mesh, especially if the aneurysm is irregularly shaped.

In an example, a device can comprise two flexible longitudinal members (e.g. wires) which are attached to different lateral (e.g. right and left) sides of an expandable mesh. In an example, a device can include a plurality of flexible longitudinal members (e.g. wires) which are connected at different locations to an expandable mesh, wherein the expandable mesh is steered in a selected direction, at a selected angle, or into a selected orientation when one or more of the flexible longitudinal members are pulled, pushed, and/or rotated.

In an example, a device can include a plurality of flexible longitudinal members (e.g. wires) which span the length of a catheter and are connected at different locations to an expandable mesh, wherein the expandable mesh is steered in a selected direction, at a selected angle, or into a selected orientation when a subset of the flexible longitudinal members are selectively pulled, pushed, and/or rotated by the device operator. In an example, a device can include a plurality of flexible longitudinal members (e.g. wires) which span the length of a catheter and are connected at different locations to an expandable mesh, wherein the expandable mesh expands in a selected radially-asymmetric manner when one or more of the flexible longitudinal members are pulled, pushed, and/or rotated.

In an example, a device can include one or more flexible longitudinal members (e.g. wires) which are connected to an expandable mesh, wherein the expandable mesh is steered in a selected direction, at a selected angle, or into a selected orientation when one or more of the flexible longitudinal members are selectively pulled, pushed, and/or rotated by a device operator. In an example, a device can include one or more flexible longitudinal members (e.g. wires) which are connected to an expandable mesh, wherein the expandable mesh expands in a selected radially-asymmetric manner when one or more of the flexible longitudinal members are pulled, pushed, and/or rotated.

In an example, a device can include one or more flexible longitudinal members (e.g. wires) which span the length of a catheter and are connected to an expandable mesh, wherein the expandable mesh expands in a selected radially-asymmetric manner when one or more of the flexible longitudinal members are selectively pulled, pushed, and/or rotated by a device operator. In an example, a device operator can manually push, pull, or rotate flexible longitudinal members to steer a mesh in a selected direction, at a selected angle, or with a selected orientation.

In an example, a device operator can steer an expandable mesh in a selected direction, at a selected angle, or into a selected orientation by selectively transmitting electricity to one or more piezoelectric members (e.g. piezoelectric cords or strands) which are connected to different locations on the mesh. In an example, a flexible longitudinal member can be a wire. In another example, a flexible longitudinal member can travel through a channel in a catheter wall. In an example, a plurality of wires can be connected to different locations (e.g. to opposite sides) of an expandable mesh, wherein selective pulling, pushing, and/or rotating one or more of the wires causes the mesh to expand in an asymmetric manner and/or steers the mesh in a selected direction, at a selected angle, or with a selected orientation.

In an example, a device can comprise an expandable mesh with multiple potential opening locations (e.g. a central location and multiple non-central locations), wherein a device operator selects which of the potential opening locations to open to insert expandable members and/or material into an aneurysm sac, and wherein the device operator opens a selected opening location by applying electrical energy that location. In an example, a device can comprise an expandable mesh with multiple potential opening locations (e.g. a central location and multiple non-central locations), wherein a device operator selects which of the potential opening locations to open to insert expandable members and/or material into an aneurysm sac, and wherein the device operator opens a selected opening location by rotating a portion of the mesh.

In an example, an opening in a multi-layer mesh through which embolic members and/or material are inserted can be formed by aligning an opening in a first layer of the mesh with an opening in a second layer of the mesh. In an example, embolic members and/or material can be inserted into an aneurysm sac through a central opening of a half-torus shaped expandable mesh. In an example, there can be a plurality of openings in an expandable mesh through which embolic members and/or material are inserted into an aneurysm sac. In another example, there can be a plurality of openings on an expandable mesh, wherein there is a valve on each of the openings, and wherein a device opening can selectively open one of the valves to insert embolic members and/or material through it. In an example, there can be an opening in a multi-layer expandable mesh through which embolic members and/or material are inserted into a gap between layers of the expandable mesh. In another example, there can be an opening in the proximal side (or surface) of an expandable mesh through which embolic members and/or material are inserted into an aneurysm sac.

In an example, a device operator can remotely control the operation of a valve in an opening through an expandable mesh using a ball valve. In another example, a device operator can remotely control the operation of a valve in an opening through an expandable mesh using a check valve. In an example, a device operator can remotely control the operation of a valve in an opening through an expandable mesh using a diaphragm valve. In an example, a device operator can remotely control the operation of a valve in an opening through an expandable mesh using a gate value. In an example, a device operator can remotely control the operation of a valve in an opening through an expandable mesh using a magnet.

In an example, a device operator can remotely control the operation of a valve in an opening through an expandable mesh using a pneumatic valve. In another example, a device operator can remotely control the operation of a valve in an opening through an expandable mesh using a seal. In an example, a device operator can remotely control the operation of a valve in an opening through an expandable mesh using a solenoid. In an example, a subset of openings can be remotely opened by a device operator via targeted transmission of thermal energy. In an example, there can be a valve in an opening through an expandable mesh through which embolic members and/or material can be inserted into an aneurysm sac, wherein a device operator can remotely open or close the valve.

In an example, there can be a valve in an opening through an expandable mesh through which embolic members and/or material can be inserted into an aneurysm sac, wherein a device operator can remotely open or close the valve by transmitting electricity to the valve. In an example, a distal mesh can have a higher proportion of polymer components than a proximal mesh. In an example, an expandable mesh can be made by braiding or weaving polymer strands, filaments, threads, or yarns. In another example, an expandable mesh can be made from one or more polymers. In an example, an expandable mesh can be made by braiding and/or weaving wires and polymer strands, filaments, and/or threads together In an example, a flexible net or mesh can be made by laser-cutting holes in a globular polymer structure (e.g. a balloon). In an example, an expandable mesh can be made by laser-cutting holes in a globular metal structure. In an example, a flexible net or mesh can be made from a thermoplastic polymer. In an example, a flexible net or mesh can be made with a polymer which has been doped or impregnated with metal particles.

In an example, one or more portions of a flexible net or mesh can be made from a radiopacifying material. In an example, one or more portions of an expandable mesh can be made from a radiopacifying material. In another example, a flexible net or mesh can be made with a polymer which has been doped or impregnated with radio-opaque and/or radiopacifying material. In an example, a catheter with a radially-asymmetric distal end portion can enable a device operator to select the direction, angle, and/or orientation by which an expandable mesh exits the catheter. In an example, a device can include a catheter with a diagonally-cut end which steers an expandable mesh in a selected direction, at a selected angle, or into a selected orientation.

In an example, a device can include a catheter with a radially-asymmetric end which causes an expandable mesh to expand in a radially-asymmetric manner. In an example, a device operator can use a catheter with a diagonal end to control radially-asymmetric expansion of an expandable mesh. In an example, a device operator can use a catheter with a radially-asymmetric end to control radially-asymmetric expansion of an expandable mesh. In another example, an end portion of a catheter can be curved at an obtuse angle like a piece of plumbing pipe. In an example, the angle of the end of a dual catheter can be changed by rotating an inner catheter relative to an outer catheter, or vice versa.

In an example, a device can further comprise one or more hydraulic pistons which move (e.g. rotated, pivot, and/or tilt) an end portion of a catheter to steer an expandable mesh in a selected direction, at a selected angle, and/or with a selected orientation. In an example, an end portion of a catheter can be connected to the rest of the catheter by a joint, wherein the end portion is moved (e.g. rotated, pivoted, and/or tilted) by the joint in order to steer an expandable mesh in a selection direction. In an example, an end portion of a catheter can be moved (e.g. rotated, pivoted, and/or tilted) in order to steer an expandable mesh in a selection direction.

In an example, a device can include a movable (e.g. rotatable, pivoting, and/or tilting) joint between an end portion of catheter and the rest of the catheter, wherein the movable joint enables a device operator to steer the expandable mesh in a selected direction, at a selected angle, or into a selected orientation. In another example, a device operator can use one or more actuators to move a joint between a catheter and an expandable mesh. In an example, a device operator can use one or more electromagnetic actuators (e.g. micromotors) to move a joint between an end portion of a catheter and the rest of the catheter.

In an example, a joint between a catheter and an expandable mesh can be moved by one or more actuators. In an example, a joint between an end portion of a catheter and the rest of the catheter can be a rotational joint (e.g. ball bearing). In an example, a joint between an end portion of a catheter and the rest of the catheter can be moved by one or more hydraulic actuators (e.g. micropistons). In an example, a movable joint can be articulated and/or pleated. In another example, a device operator can change a direction, angle, and/or orientation of an expandable mesh by selectively moving (e.g. pivoting and/or rotating) a movable joint between an end portion of a catheter and the rest of the catheter.

In an example, a device can a coaxial catheter, wherein there are one or more wires or cords between an inner catheter and outer catheter in the coaxial catheter, and wherein pulling or pushing the one or more wires or cords causes an expandable mesh to expand in a radially-asymmetric manner. In an example, a device can include a coaxial catheter which further comprises an inner catheter and an outer catheter. In an example, a device can include a coaxial catheter which further comprises an inner catheter with a radially-symmetric distal end and an outer catheter with a radially-asymmetric distal end. In an example, a device can include a dual catheter wherein the dual catheter further comprises first and second catheters which are parallel to each other.

In an example, a device can include a catheter that delivers embolic members and/or embolic material into an aneurysm sac. In another example, a device can include a catheter that delivers embolic members and/or embolic material through a opening in a proximal side (or surface) of expandable mesh. In an example, a device can include a catheter that is inserted into an aneurysm sac. In an example, a device can include a catheter with a radially-asymmetric end portion which a device operator can use to steer an expandable mesh in a selected direction, at a selected angle, or into a selected orientation. In an example, a catheter can deliver an expandable mesh into an aneurysm sac. In an example, a catheter can deliver embolic members and/or material into an aneurysm sac via a pusher wire. In an example, a device can further comprise a liquid flow (e.g. saline flow) which is used to deliver an expandable mesh through a catheter to an aneurysm sac. In an example, a device can further comprise a pusher wire or plunger which is to deliver embolic members and/or material through a catheter to an aneurysm sac.

In an example, embolic members and/or material can comprise coils (e.g. metal coils). In an example, embolic members and/or material can comprise liquid or gel (which congeals after insertion into an aneurysm sac). In an example, embolic members and/or material can comprise microspheres. In an example, embolic members and/or material can comprise springs. In an example, embolic components (e.g. beads, microspheres, microsponges, or hydrogels) in a string-of-pearls embolic strand can be centrally connected along a flexible longitudinal member (e.g. wire, suture, string, cord, filament, coil, or spring). In an example, embolic components (e.g. beads, microspheres, microsponges, or hydrogels) in a string-of-pearls embolic strand can be connected by a flexible longitudinal member (e.g. wire, suture, string, cord, filament, coil, or spring) which passes through openings in their centers. In an example, distal embolic components (e.g. beads, microspheres, microsponges, or hydrogels) in a string-of-pearls embolic strand can have higher durometer and/or Shore values than proximal embolic components on the strand.

In an example, an embolic member can be a string-of-pearls embolic strand, wherein an string-of-pearls embolic strand comprises a longitudinal series (or sequence) of embolic components (e.g. beads, microspheres, microsponges, hydrogels, polyhedrons, or polymer masses) connected (in a pairwise manner) by a flexible longitudinal member (e.g. wire, suture, thread, string, cord, or coil). In another example, distal embolic components (e.g. beads, microspheres, microsponges, hydrogels, polyhedrons, or polymer masses) in a string-of-pearls embolic strand can be larger than proximal embolic components in the strand. In an example, embolic components in a string-of-pearls embolic strand can connect, attach, and/or adhere to each other when they contact (e.g. press against) each other during deployment in an aneurysm sac.

In an example, embolic components and/or material can be inserted into an aneurysm sac through one or more openings in an expandable mesh. In an example, there can be a central opening in the proximal side (or surface) of an braided or woven expandable mesh through which embolic members and/or material are inserted into an aneurysm sac, wherein the central opening is larger than openings or pores in the braid or weave of the rest of the expandable mesh. In an example, there can be a plurality of openings in the proximal side (or surface) of an expandable mesh, wherein a device operator can select through which of these openings to insert embolic members and/or material into an aneurysm sac.

In an example, a device can further comprise fluid-filled expandable components in or on an expandable mesh, wherein selectively filling one or more of the fluid-filled expandable components more than other expandable components steers the expandable mesh in a selected direction, at a selected angle, or into a selected orientation. In an example, a device can further comprise fluid-filled expandable components in or on an expandable mesh, wherein a device operator can cause and/or control asymmetric expansion of the expanded mesh by selectively filling one or more of the fluid-filled expandable components more than other expandable components. In an example, a device operator can control radially-asymmetric expansion of an expandable mesh by inserting different amounts of flowable substance into different expandable components in the mesh. In an example, a multi-layer expandable mesh can further comprise a plurality of fluid-filled components between layers of the mesh, wherein selectively filling a first component to a greater extent than filling a second component causes radially-asymmetric expansion of the expandable mesh.

In an example, a device operator can fill different chambers in an expandable mesh with different amounts of embolic members or material in order to cause and/or control radially-asymmetric expansion of the expandable mesh. In an example, an expandable mesh can further comprise a plurality of inner chambers which can be selectively filled with embolic members or material, wherein selective filling of some of the chambers more than other chambers causes some portions of the expandable mesh to expand more than other portions of the expandable mesh.

In an example, a device can comprise a plurality (e.g. longitudinal series) of wire or coil loops which are inserted into an aneurysm sac, wherein adjacent loops are connected to each other at a single locations. In an example, a device can comprise a plurality (e.g. longitudinal series) of wire or coil loops which are inserted into an aneurysm sac, wherein the loops are created from a single continuous wire or coil. In an example, a device can include a wire and/or coil loop which is inserted into an aneurysm sac. In an example, a device can include a wire and/or coil loop which is inserted into an aneurysm sac, wherein the wire and/or coil loop has two branches (e.g. sides) which are parallel to each other as they are delivered through a catheter to the aneurysm sac, and wherein a curved end connecting the two branches is the first portion of the loop which is inserted into the aneurysm sac.

In an example, a device can include a wire and/or coil loop which is inserted into an aneurysm sac, wherein the wire and/or coil loop has two branches (e.g. sides) which are parallel to each other as they are delivered through a catheter to the aneurysm sac, and wherein a device operator can steer the loop in a selected direction, at a selected angle, or into a selected orientation by selectively pushing one branch of the loop farther into the aneurysm sac than the other branch of the loop. In an example, a device operator can selectively steer a wire or coil loop in different directions by non-uniform insertion of first and second branches of the loop into an aneurysm sac. In an example, two branches (e.g. side) of a wire or coil loop can be delivered to an aneurysm sac through two different (but parallel) catheters.

In an example, distal wire or coil loops in a longitudinal series of loops can be larger than proximal wire or coil loops in the series. In an example, the plane of a wire or coil loop can be within 15 degrees of being parallel to the plane of a central circumference of an aneurysm sac. In an example, wire or coil loops which are inserted into an aneurysm sac can accumulate into a mass with a zigzag pattern.

In an example, a device can comprise a pneumatic actuator (e.g. inflatable chamber). In an example, a device can comprise electroconductive pathways (e.g. wires) along a catheter which provide power to one or more actuators at the end of the catheter. In an example, a device can comprise one or more actuators at the end of a catheter. In an example, a device can comprise one or more actuators which move (e.g. rotate, pivot, and/or tilt) an expandable mesh. In an example, a device can comprise one or more actuators which a device operator uses to cause an expandable mesh to expand in a radially-asymmetric manner. In an example, a device can comprise one or more actuators which push or pull longitudinal members (e.g. wires or cords) which are attached to different locations on an expandable mesh. In an example, a device can comprise one or more actuators.

In an example, an intrasaccular aneurysm occlusion device can comprise: (a) an expandable mesh (e.g. mesh, lattice, braid, stent, and/or neck bridge) that is inserted and expanded within an aneurysm sac, wherein the expandable mesh is expanded in a radial manner at a first time within the aneurysm sac into a first configuration having a convex (e.g. globular, spherical, ellipsoidal, or oblate spheroidal) shape; wherein the expandable mesh is compressed in a distal-to-proximal manner at a second time within the aneurysm sac into a second configuration having a concave (e.g. bowl, cup, hemispherical, hemi-ellipsoidal, or funnel) shape; wherein the expandable mesh further comprises a (central) annular section (or band) which is more elastic and/or flexible than the rest of the expandable mesh; wherein a first portion (e.g. first third or half) of the expandable mesh is distal relative to the annular section (or band) in the first configuration; wherein a second portion (e.g. second third or half) of the expandable mesh is proximal relative to the annular section (or band) in the first configuration; wherein the first portion of the expandable mesh is compressed, inverted, and/or nested into the second portion of the expandable mesh as the expandable mesh is changed from the first configuration to the second configuration; wherein both the first portion of the expandable mesh and the second portion of the expandable mesh are proximal relative to the annular section (or band) in the second configuration; (b) embolic components and/or material (e.g. beads, microspheres, microsponges, hydrogels, congealing liquid or gel, or embolic coils) that are inserted into portions of the aneurysm sac which are distal to the expandable mesh, and wherein insertion of the embolic components and/or material into the aneurysm sac compresses (e.g. pressures or compels) the expandable mesh from the first configuration having the convex shape to the second configuration having the concave shape; and (c) one or more catheters which deliver the expandable mesh and/or the embolic components and/or material to the aneurysm sac.

In an example, a distal portion (e.g. distal third or half) of a convex expandable mesh can be collapsed, compressed, folded, and/or inverted into a concavity of a proximal portion (e.g. proximal third or half) of the expandable mesh within an aneurysm sac. In an example, a distal portion (e.g. distal third or half) of a convex expandable mesh can be collapsed, compressed, folded, and/or inverted along an flexible annular section (or band) of the mesh into a concavity of a proximal portion (e.g. proximal third or half) of the expandable mesh.

In an example, an expandable mesh which is inserted into an aneurysm can have a first configuration and a second configuration, wherein the first configuration is convex (e.g. globular) and the second configuration is concave (e.g. bowl shaped), wherein the mesh expands into the first configuration in an aneurysm sac after it exits a catheter, and wherein the mesh is collapsed, compressed, folded, and/or inverted into the second configuration after expanding into the first configuration. In an example, when an expandable mesh is in a convex (e.g. globular) configuration, the expandable mesh can comprise a distal portion (e.g. distal third or third or half), a proximal portion (e.g. proximal third or third or half), and flexible annular section (or band) around a (central) circumference between the distal portion and the proximal portion, wherein the distal portion is collapsed, compressed, and/or inverted into a concavity of the proximal portion.

In an example, an expandable mesh can expand into a globular (e.g. spherical, ellipsoidal, or oblate spheroidal) shape in an aneurysm sac and then be collapsed, compressed, and/or inverted into a concave (e.g. bowl, cup, hemispherical, hemi-ellipsoidal) shape by pressure from the accumulation of embolic members and/or material in a distal portion of the aneurysm sac. In an example, an expandable mesh can be changed from a convex (e.g. globular) shape to a concave (e.g. bowl) shape by collapsing, compressing, and/or inverting a distal portion of the mesh into a concavity of a proximal portion of the mesh by pressure on the distal portion of the mesh from accumulation of embolic members and/or material in an aneurysm sac.

In an example, a distal portion (e.g. distal third or half) of a convex expandable mesh can be collapsed, compressed, and/or inverted along an elastic and/or flexible annular section (or band) of the mesh into a concavity of a proximal portion (e.g. proximal third or half) of the expandable mesh by the insertion of embolic members and/or material into an area of an aneurysm sac which is distal relative to the expandable mesh. In an example, when an expandable mesh is in a convex configuration, the expandable mesh can comprise a distal portion (e.g. distal third or third or half), a proximal portion (e.g. proximal third or third or half), a flexible annular section (or band) around a (central) circumference between the distal portion and the proximal portion, and a wire or cord attached to the distal portion, wherein the distal portion is collapsed, compressed, and/or inverted into a concavity of the proximal portion by pressure from the accumulation of embolic members and/or material in (a distal area of) an aneurysm sac.

In an example, a first portion of an expandable mesh can be distal to a second portion of the expandable mesh in a first configuration, but the first portion can be nested within a concavity of the second portion in a first configuration after the expandable mesh has been collapsed, compressed, and/or inverted in a distal-to-proximal direction. In an example, an expandable mesh can have a single-layer convex first configuration and a two-layer concave second configuration, wherein the expandable mesh has a distal portion (e.g. distal third or half) and a proximal portion (e.g. proximal third or half) in the convex first configuration, wherein the distal portion is collapsed, compressed, and/or inverted into the proximal portion in the second configuration, and wherein the distal portion is nested within a concavity of the proximal portion in the second configuration.

In an example, an expandable mesh which is inserted into an aneurysm sac can comprise: a first portion (e.g. first third or half) which is a distal portion when the mesh is in an expanded convex configuration; a second portion (e.g. second third or half) which is a proximal portion when the mesh is in the expanded convex configuration; and a flexible section between the first portion and the second portion, wherein the flexible section spans a (central) perimeter of the expandable mesh, and wherein the first portion is collapsed, compressed, and/or inverted around the flexible section into a concavity of the second portion.

In an example, an expandable mesh can comprise a first concave portion which opens proximally, a second concave portion which opens distally, and an annular section (or band) between the first and second concave portions, wherein the expandable mesh has a first configuration in which the first concave portion is distal relative to the annular section (or band) and the second concave portion is proximal relative to the annular section (or band), and wherein the expandable mesh has a second configuration in which the first concave portion is inverted into the second concave portion and both the first and second concave portions are proximal relative to the annular section (or band).

In an example, an expandable mesh can be changed from a convex (e.g. globular) shape to a concave (e.g. bowl) shape by collapsing, compressing, and/or inverting a distal portion of the mesh into a concavity of a proximal portion of the mesh by pulling a wire attached to the distal portion. In an example, when an expandable mesh is in a convex configuration, the expandable mesh can comprise a distal portion (e.g. distal third or third or half), a proximal portion (e.g. proximal third or third or half), a flexible annular section (or band) around a (central) circumference between the distal portion and the proximal portion, and a wire or cord attached to the distal portion, wherein the distal portion is collapsed, compressed, and/or inverted into a concavity of the proximal portion when the wire or cord is pulled in a proximal direction.

In an example, when an expandable mesh is in a convex configuration, the expandable mesh can comprise a distal portion (e.g. distal third or third or half), a proximal portion (e.g. proximal third or third or half), a flexible annular section (or band) around a (central) circumference between the distal portion and the proximal portion, and a wire attached to the distal portion, wherein the distal portion is collapsed, compressed, and/or inverted into a concavity of the proximal portion when the wire or cord is pulled by a device operator.

In an example, an expandable mesh can expand into a globular (e.g. spherical, ellipsoidal, or oblate spheroidal) shape in an aneurysm sac and then be collapsed, compressed, and/or inverted into a concave (e.g. bowl, cup, hemispherical, hemi-ellipsoidal) shape. In an example, an expandable mesh can have a first convex (e.g. globular, spherical, ellipsoidal, or oblate spheroidal) shape and a second concave (e.g. bowl, cup, hemispherical, or funnel) shape, wherein the mesh is collapsed, compressed, and/or inverted from the first shape to the second shape by pressure from accumulation of embolic members and/or material in an aneurysm sac. In an example, an expandable mesh can have a first convex (e.g. globular, spherical, ellipsoidal, or oblate spheroidal) shape and a second concave (e.g. bowl, cup, hemispherical, or funnel) shape, wherein the mesh is collapsed, compressed, and/or inverted from the first shape to the second shape by pulling a wire or cord attached to a distal portion of the mesh.

In an example, an expandable mesh can be made by braiding or weaving metal wires or tubes. In an example, an expandable mesh can be formed by braiding or weaving polymer strands. In an example, an expandable mesh can be formed by weaving or braiding metal wires (or tubes) and polymer strands together. In an example, an expandable mesh can be made by 3D printing. In an example, an expandable mesh can radially self-expand within an aneurysm sac. In an example, an expandable mesh can have a globular and/or spherical shape in a first configuration. In an example, an expandable mesh can have an ellipsoidal or oblate spheroidal shape in a first configuration. In an example, an expandable mesh can have a half-torus shape in a first configuration.

In an example, an expandable mesh can have a concave bowl or cup shape in a second configuration. In an example, an expandable mesh can have a funnel or half-hyperbolic shape in a second configuration. In an example, an expandable mesh can have a hemispherical or hemi-ellipsoidal concave shape in a second configuration. In an example, expandable mesh can be expanded in a radially-asymmetric manner by a device operator in order to steer it in a selected direction, at a selected angle, or with a selected orientation. In an example, a single-layer distal portion (e.g. distal third or half) of a convex expandable mesh can be compressed into a concavity of a proximal portion (e.g. proximal third or half) of the expandable mesh to form a two-layer convex expandable mesh within an aneurysm sac.

In an example, an expandable mesh can comprise a first set of wires, tubes, strands, or filaments with a first orientation (e.g. a circumferential orientation) and a second set of wires, tubes, strands, or filaments with a second orientation (e.g. a proximal-to-distal orientation), wherein wires, tubes, strands, or filaments in the first set are thinner or more sparsely configured in a flexible annular section (or band) of the expandable mesh than in the rest of the expandable mesh (e.g. in distal and proximal portions of the annular mesh). In an example, an expandable mesh can comprise a first set of wires, tubes, strands, or filaments with a first orientation (e.g. a circumferential orientation) and a second set of wires, tubes, strands, or filaments with a second orientation (e.g. a proximal-to-distal orientation), wherein wires, tubes, strands, or filaments in the second set are thinner or more sparsely configured in a flexible annular section (or band) of the expandable mesh than in the rest of the expandable mesh (e.g. in distal and proximal portions of the annular mesh).

In an example, a flexible annular section (or band) around a circumference of an expandable mesh can span a central circumference of the expandable mesh. In an example, a flexible section of an expandable mesh can be around a central perimeter of the expandable mesh. In an example, an expandable band or ring can be around a central circumference of an expandable mesh. In an example, a flexible annular section (or band) around a circumference of an expandable mesh can span a widest circumference of the expandable mesh.

In an example, an expandable mesh which is inserted into an aneurysm sac can comprise: a first portion (e.g. first third or half) which is a distal portion when the mesh is in an expanded convex configuration; a second portion (e.g. second third or half) which is a proximal portion when the mesh is in the expanded convex configuration; and a flexible section between the first portion and the second portion, wherein the flexible section spans a (central) perimeter of the expandable mesh.

In an example, a flexible annular section (or band) of an expandable mesh can have a circular cross-sectional shape. In an example, a flexible annular section (or band) of an expandable mesh can be circumferential. In an example, a cross-section of a flexible annular section (or band) of an expandable mesh can have a convex shape. In an example, a flexible annular section (or band) of an expandable mesh can have an elliptical cross-sectional shape. In an example, a cross-section of a flexible annular section (or band) of an expandable mesh can have an undulating (e.g. sinusoidal) circular shape. In an example, a cross-section of a flexible annular section (or band) of an expandable mesh can have an undulating (e.g. sinusoidal) convex shape. In an example, a flexible annular section (or band) of an expandable mesh can have an undulating (e.g. sinusoidal) cross-sectional shape.

In an example, a flexible annular section (or band) around a (central) perimeter of an expandable mesh can have a variable-width around its circumference. In an example, a flexible annular section (or band) of an expandable mesh can have a uniform proximal-to-distal width. In an example, a flexible annular section (or band) around a (central) perimeter of an expandable mesh can be radially asymmetric. In an example, a flexible annular section (or band) around a (central) circumference of an expandable mesh can have a proximal-to-distal width which is less than 2 mm. In an example, a flexible annular section (or band) around a (central) circumference of an expandable mesh can have a proximal-to-distal width between 1 mm and 3 mm. In an example, a flexible annular section (or band) around a (central) circumference of an expandable mesh can have a proximal-to-distal width between 2 mm and 5 mm.

In an example, a proximal-to-distal width of an annular section (or band) of an expandable mesh can be between 5% and 15% of the length of a proximal-to-distal axis of the mesh when the mesh is in a convex first configuration. In an example, a proximal-to-distal width of an annular section (or band) of an expandable mesh can be between 10% and 25% of the length of a proximal-to-distal axis of the mesh when the mesh is in a convex first configuration. In an example, an annular section (or band) around a (central) circumference of an expandable mesh can comprise between 2% and 10% of the (surface area of the) expandable mesh. In an example, an annular section (or band) around a (central) circumference of an expandable mesh can comprise between 5% and 20% of the (surface area of the) expandable mesh.

In an example, an elastic and/or flexible annular section (or band) around the central circumference of an expandable mesh can comprise between 5% and 20% of the surface area of the expandable mesh. In an example, an elastic and/or flexible annular section (or band) can comprise around the central circumference of an expandable mesh can comprise between 5% and 20% of the height of aneurysm sac (e.g. from the aneurysm neck to the aneurysm dome). In an example, an expandable mesh which is inserted into an aneurysm can comprise: a distal portion of an expandable mesh, a proximal portion of the expandable mesh, and a flexible annular portion of the expandable mesh, wherein the flexible annular portion is between the distal portion and the proximal portion, wherein the distal portion spans a first percentage (e.g. between 25% and 50%) of the proximal-to-distal axis of the expandable mesh when the mesh is in an expanded convex configuration, wherein the proximal portion spans a second percentage (e.g. between 25% and 50%) of the proximal-to-distal axis of the expandable mesh when the mesh is in the expanded convex configuration, and wherein the flexible annular portion spans the remaining percentage (e.g. 100% minus the first and second percentages) of the proximal-to-distal axis.

In an example, an expandable mesh which is inserted into an aneurysm can comprise: a distal portion of an expandable mesh, a proximal portion of the expandable mesh, and a flexible annular portion of the expandable mesh, wherein the flexible annular portion is between the distal portion and the proximal portion, wherein the distal portion spans a first percentage (e.g. between 30% and 45%) of the proximal-to-distal axis of the expandable mesh when the mesh is in an expanded convex configuration, wherein the proximal portion spans a second percentage (e.g. between 30% and 45%) of the proximal-to-distal axis of the expandable mesh when the mesh is in the expanded convex configuration, and wherein the flexible annular portion spans the remaining percentage (e.g. 100% minus the first and second percentages) of the proximal-to-distal axis.

In an example, an annular section (or band) around a central circumference of an expandable mesh can be more flexible than the rest of the mesh. In an example, a flexible annular section (or band) around a (central) circumference of an expandable mesh can be more elastic than distal or proximal portions of the expandable mesh. In an example, a flexible annular section (or band) around a (central) circumference of an expandable mesh can be more elastic than the rest of the expandable mesh. In an example, a flexible annular section (or band) around a (central) circumference of an expandable mesh can be more elastic and/or stretchable than the rest of the expandable mesh. In an example, a flexible annular section (or band) around a (central) circumference of an expandable mesh can be more elastic and/or stretchable than distal or proximal portions of the expandable mesh.

In an example, an annular section (or band) around a central circumference of an expandable mesh can be more elastic than the rest of the mesh. In an example, an annular section (or band) of an expandable mesh can be made from material which is more elastic and/or more flexible than material used to make the rest of the expandable mesh. In an example, wires, tubes, or fibers comprising an annular section (or band) of an expandable mesh can be made with one or more materials which are more elastic and/or flexible than the one or more materials used to make the wires, tubes, or fibers comprising the rest of the expandable mesh.

In an example, a flexible annular section (or band) around a (central) circumference of an expandable mesh can be made with lower durometer material than the rest of the expandable mesh. In an example, a flexible annular section (or band) around a (central) circumference of an expandable mesh can be made with lower durometer material than distal or proximal portions of the expandable mesh. In an example, an annular section (or band) around a central circumference of an expandable mesh can have a lower durometer and/or Shore value than the rest of the mesh. In an example, a flexible annular section (or band) around a (central) circumference of an expandable mesh can have a less dense mesh, weave, or braid than the rest of the expandable mesh. In an example, a flexible annular section (or band) around a (central) circumference of an expandable mesh can have a less dense mesh, weave, or braid than distal or proximal portions of the expandable mesh.

In an example, a flexible annular section (or band) around a (central) circumference of an expandable mesh can be thinner (and/or have thinner mesh components) than the rest of the expandable mesh. In an example, a flexible annular section (or band) around a (central) circumference of an expandable mesh can be thinner (and/or have thinner mesh components) than distal or proximal portions of the expandable mesh. In an example, wires, tubes, or fibers comprising an annular section (or band) of an expandable mesh can be thinner and/or smaller than wires, tubes, or fibers comprising the rest of the expandable mesh.

In an example, an expandable mesh can comprise a weave or braid of a first set of wires, tubes, filaments, or strands with a first orientation and a second set of wires, tube, filaments, or strands with a second orientation, wherein the first and second sets of wires, tubes, filaments, or strands intersect to form the weave or braid, and wherein a flexible annular section, or band) around a (central) circumference of the expandable mesh comprises only one of the sets of wires, tubes, filaments, or strands. In an example, an expandable mesh can comprise a weave or braid of a first set of wires, tubes, filaments, or strands with a first orientation and a second set of wires, tube, filaments, or strands with a second orientation, wherein the first and second sets of wires, tubes, filaments, or strands intersect to form the weave or braid, and wherein a flexible annular section (or band) around a (central) circumference of the expandable mesh has a different ratio, proportion, and/or blend of the first and second sets than the rest of the expandable mesh.

In an example, an expandable mesh can comprise a weave or braid of a first set of wires, tubes, filaments, or strands with a circumferential shape or orientation and a second set of wires, tube, filaments, or strands with a longitudinal shape or orientation, wherein the first and second sets of wires, tubes, filaments, or strands intersect to form the weave or braid, and wherein a flexible annular section (or band) around a (central) circumference of the expandable mesh has a different ratio, proportion, and/or blend of the first and second sets than the rest of the expandable mesh.

In an example, braid or weave of wires, tubes, or fibers comprising an annular section (or band) of an expandable mesh can be more sparse, less dense, more open, and/or more loose than a braid or weave of wires, tubes, or fibers comprising the rest of the expandable mesh. In an example, the braid or weave of wires, tubes, or fibers comprising an annular section (or band) of an expandable mesh can be more sparse, less dense, more open, and/or more loose than that of the rest of the mesh. In an example, wires, tubes, and/or strands used to form an annular section (or band) around a central circumference of an expandable mesh can be more flexible, more elastic, thinner, smaller, and/or woven less densely than those in rest of the mesh.

In an example, an expandable mesh can comprise an annular section (or band) around a (central) conference of the mesh, wherein the annular section (or band) is more flexible, more elastic, and/or thinner than the rest of the mesh so that a distal portion (e.g. distal third or half) of the collapses, compresses, and/or inverts into a proximal portion (e.g. proximal third or half) of the mesh when pressure is applied to the distal portion. In an example, an expandable mesh can comprise an annular section (or band) around a (central) conference of the mesh, wherein the annular section (or band) is more flexible, more elastic, and/or thinner than the rest of the mesh so that a distal portion (e.g. distal third or half) of the mesh collapses, compresses, and/or inverts into a proximal portion (e.g. proximal third or half) of the mesh when pressure is applied to the distal portion by the accumulation of embolic members and/or embolic material in an aneurysm sac.

In an example, an expandable mesh which is inserted into an aneurysm sac can comprise: a distal portion (e.g. distal third) of the expandable mesh; a proximal portion (e.g. proximal third) of the expandable mesh; and a central portion (e.g. central third) of the expandable mesh; wherein the central portion is annular (e.g. around the central circumference of the expandable mesh); wherein the central portion is more flexible, more elastic, less dense, and/or thinner than the distal and proximal portions; and wherein the distal portion is collapsed, compressed, and/or inverted into a concavity of the proximal portion by pressure from an accumulation of embolic members and/or material in the aneurysm sac. In an example, an expandable mesh which is inserted into an aneurysm sac can comprise: a distal portion (e.g. distal third) of the expandable mesh; a proximal portion (e.g. proximal third) of the expandable mesh; and a central portion (e.g. central third) of the expandable mesh; wherein the central portion is annular (e.g. around the central circumference of the expandable mesh); wherein the central portion is more flexible, more elastic, less dense, and/or thinner than the distal and proximal portions; and wherein the distal portion is collapsed, compressed, and/or inverted into a concavity of the proximal portion.

In an example, a flexible annular section (or band) around a (central) circumference of an expandable mesh can be weakened remotely by a device operator. In an example, a flexible annular section (or band) around a (central) circumference of an expandable mesh can be weakened remotely by a device operator by transmission of electrical energy to the annular section (or band). In an example, selected components of a flexible annular section (or band) around a (central) circumference of an expandable mesh can be weakened (e.g. melted and/or detached) remotely by transmission of electrical energy. In an example, selected components of a flexible annular section (or band) around a (central) circumference of an expandable mesh can be weakened (e.g. melted and/or detached) remotely by exposure to light energy. In an example, selected components of a flexible annular section (or band) around a (central) circumference of an expandable mesh can be weakened (e.g. melted and/or detached) remotely by sonic energy.

In an example, embolic components and/or material can be inserted into an aneurysm sac through one or more openings in an expandable mesh. In an example, there can be one or more openings (e.g. holes or valves) in an expandable mesh through which embolic members and/or material are inserted to the aneurysm sac. In an example, embolic members and/or material can be inserted through an opening in an expandable mesh into the dome of an aneurysm sac, wherein pressure from the accumulation of the embolic members and/or material collapses, compresses, and/or inverts a distal portion (e.g. distal third or half) of the mesh into a concavity of a proximal portion of the mesh. In an example, embolic members and/or material can be inserted through a funnel-shaped opening in an expandable mesh into the dome of an aneurysm sac, wherein pressure from the accumulation of the embolic members and/or material collapses, compresses, and/or inverts a distal portion (e.g. distal third or half) of the mesh into a concavity of a proximal portion of the mesh.

In an example, an opening valve and/or closure mechanism can be selected from the group consisting of: adhesive, ball valve, butterfly valve, cap, check valves, clamp, clip, diaphragm valve, drawstring, fusible member, gate value, globe valve, loop, magnet, pinch valve, plug, plug valve, pneumatic valve, pull cord, pull wire, seal, sliding valve, snap, solenoid, and threaded valve. In an example, embolic members and/or material can be inserted through an opening in an expandable mesh into the dome of an aneurysm sac, wherein pressure from the accumulation of the embolic members and/or material collapses, compresses, and/or inverts a distal portion (e.g. distal third or half) of the mesh into a concavity of a proximal portion of the mesh, wherein there is a one-way valve in the opening.

In an example, embolic members and/or material can be inserted through an opening in an expandable mesh into the dome of an aneurysm sac, wherein pressure from the accumulation of the embolic members and/or material collapses, compresses, and/or inverts a distal portion (e.g. distal third or half) of the mesh into a concavity of a proximal portion of the mesh, wherein there is a leaflet (e.g. tri-leaflet) valve in the opening. In an example, embolic members and/or material can be inserted through an opening in an expandable mesh into the dome of an aneurysm sac, wherein pressure from the accumulation of the embolic members and/or material collapses, compresses, and/or inverts a distal portion (e.g. distal third or half) of the mesh into a concavity of a proximal portion of the mesh, wherein there is a valve in the opening which the device operator can open or close remotely.

In an example, there can be an opening on a proximal side (or surface) of an expandable mesh through which embolic components and or material are inserted, wherein there is also a closure mechanism which a device operator can control remotely to selectively open or close the opening. In an example, embolic members and/or material can be inserted through a gap between an expandable mesh and a wall of an aneurysm sac. In an example, embolic members and/or material can be inserted past an expandable mesh into an aneurysm sac through a gap (e.g. space) between the expandable mesh and the wall of the aneurysm sac.

In an example, accumulation of embolic components and/or material in a portion of an aneurysm sac which is distal to an expandable mesh can pressure, push, compel, and/or collapse a distal portion (e.g. distal third or half) of the expandable mesh into a concavity of a proximal portion (e.g. proximal third or half) of the expandable mesh. In an example, accumulation of embolic components and/or material in a portion of an aneurysm sac which is distal to an expandable mesh can pressure, push, compel, and/or collapse a distal portion (e.g. distal third or half) of the expandable mesh from a single-layer convex shape into a two-layer concave shape.

In an example, embolic components and/or material (e.g. beads, microspheres, microsponges, hydrogels, congealing liquid or gel, or embolic coils) can be are inserted into portions of the aneurysm sac which are distal to an expandable mesh. In an example, embolic components and/or material can be congealing liquid or gel. In an example, embolic components and/or material can be liquid or gel which congeals (e.g. solidifies) after insertion into an aneurysm sac. In an example, embolic components and/or material can be microsponges or hydrogels. In an example, embolic components and/or material can comprise embolic beads, microspheres, or microsponges. In an example, embolic components and/or material can comprise metal coils.

In an example, embolic components and/or material can comprise polymer coils, strands, or ribbons. In an example, embolic components and/or material can comprise string-of-pearls strands (e.g. longitudinal series of embolic pieces connected by wires, sutures, strings, cords, springs, or coils). In an example, embolic components or material can be selected from the group consisting of: embolic beads, microspheres, microsponges, hydrogels, metal coils, polymer coils, ribbons, string-of-pearls strands, congealing liquid, and congealing gel. In an example, embolic components and/or material can be conveyed through a catheter by a pusher wire and/or plunger. In an example, embolic components and/or material can be conveyed through a catheter by a liquid flow. In an example, embolic components and/or material can be conveyed through a catheter by a moving conveyor belt and/or wire loop. In an example, embolic components and/or material can be conveyed through a catheter by a rotating helix (e.g. Archimedes screw).

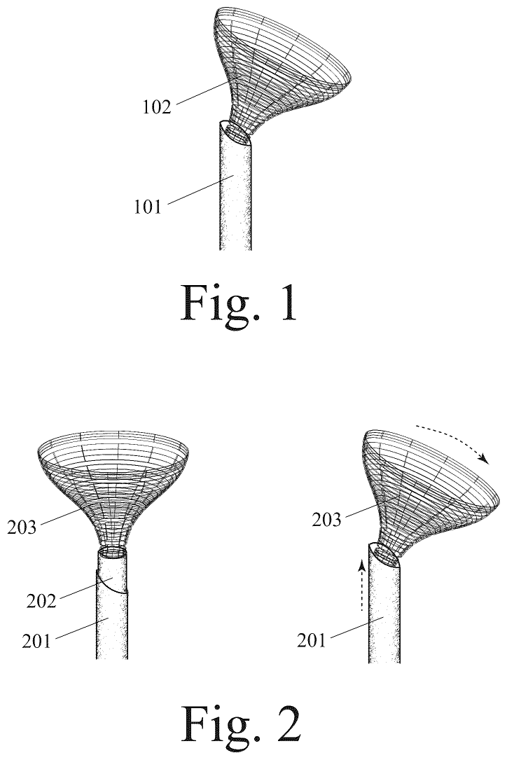

FIG. 1 shows an intrasaccular aneurysm occlusion device comprising: an expandable mesh (e.g. mesh, lattice, braid, stent, and/or neck bridge) 102 that is inserted and expanded within an aneurysm sac; and a catheter 101 with a radially-asymmetric distal end portion that is inserted into the aneurysm sac, wherein the expandable mesh is inserted through the catheter into the aneurysm sac, and wherein the distal end portion of the catheter directs (e.g. steers, compels, or pushes) the expandable mesh out of the catheter in a selected direction, at a selected angle, and/or with a selected orientation.

In an example, a selected direction may not be straight out of the catheter. In an example, a selected direction may not be colinear with a virtual extension of the central longitudinal axis of the catheter. In an example, a radially-asymmetric distal end portion of a catheter can direct (e.g. steer, compel, or push) a central axis of an expandable mesh along a vector which is not colinear with a virtual extension of the central longitudinal axis of the catheter.

In an example, a device operator can change the selected direction, angle, and/or orientation by which an expandable mesh exits a catheter. In an example, a device operator can change the selected direction, angle, and/or orientation by which an expandable mesh exits a catheter by rotating the catheter. In an example, a device operator can change a selected direction, angle, and/or orientation of the central axis of an expandable mesh by rotating a catheter. In an example, a device operator can remotely change the direction, angle, and/or orientation of an expandable mesh by rotating a proximal end of a catheter outside a person's body.

In an example, a catheter with a radially-asymmetric distal end portion can enable a device operator to select the direction, angle, and/or orientation by which a mesh exits a catheter and expands within an aneurysm sac. In an example, a device operator can change the selected direction, angle, and/or orientation of an expandable mesh by rotating the (proximal end of the) catheter from outside a person's body. In an example, the vector of a central axis of an expandable mesh can be changed by rotating a catheter having a radially-asymmetric distal end portion

In an example, a radially-asymmetric distal end portion of a catheter can be angled. In an example, a distal end portion can be formed by cutting across a catheter at an acute angle relative to the longitudinal axis of the catheter. In an example, the plane of the circumference of the distal end of a catheter can intersect the central longitudinal axis of the catheter at an acute angle. In an example, this angle can be between 25 and 75 degrees. In an example, a radially-asymmetric distal end portion of a catheter can be tilted an acute angle. In an example, this angle can be between 25 and 75 degrees relative to a central longitudinal axis of the catheter. In an example, a radially-asymmetric distal end portion of a catheter can be curved to form an acute angle (like a piece of plumbing pipe).

In an example, one section of the circumference of the distal end portion of the catheter can extend out further in a distal direction than other portions of this circumference. In an example, such differential extension can direct (e.g. steer, compel, and/or push) an expandable mesh in a selected direction, at a selected angle, or with a selected orientation as the mesh exits the catheter. In an example, a section of the circumference can span between 30% and 50% of the circumference of a distal end portion. In an example, contact between an extended section of a distal portion of a catheter can direct (e.g. steer, compel, or push) an expandable mesh out of the catheter in a selected direction, at a selected angle, and/or with a selected orientation. In an example, a distal end portion of the catheter can direct (e.g. steer, compel, or push) an expandable mesh to the right or left, or up and down, depending on how the catheter is rotated by a device operator.

In an example, one section (e.g. comprising 20% to 50%) of the circumference of a radially-asymmetric end portion of a catheter can extend out distally relative to other sections of this circumference. In an example, one section (e.g. comprising 20% to 50%) of the circumference of a radially-asymmetric end portion of a catheter can protrude radially-inward relative to other sections of this circumference. In an example, a section of the end perimeter of a distal end portion of a catheter can be curved and/or bowed radially inward. In an example, an end portion of a catheter wall which protrudes radially inward can steer (e.g. steer, compel, or push) an expandable mesh to exit the catheter in a selected direction, angle, and/or orientation. In an example, the perimeter of the end of a catheter can have a non-convex shape. In an example, the perimeter of the end of a catheter can have a cardioid and/or kidney bean shape.

In an example, an expandable mesh (e.g. mesh, lattice, braid, stent, and/or neck bridge) can expand within an aneurysm sac into a bowl, cup, hemispherical, or hemi-elliptical shape. In an example, an expandable mesh can expand within an aneurysm sac into a shape which is section (e.g. the lower half) of a torus. In an example, an expandable mesh can expand within an aneurysm sac into a globular, spherical, or elliptical shape. In an example, an expandable mesh can expand within an aneurysm sac into a funnel or parabolic shape. In an example, an expandable mesh can expand within an aneurysm sac into a hyperbolic or hourglass shape. Relevant variations discussed elsewhere in this disclosure or in priority-linked disclosures can also be applied to this example.

FIG. 2 shows two views, at two different times, of an intrasaccular aneurysm occlusion device comprising: an expandable mesh (e.g. mesh, lattice, braid, stent, and/or neck bridge) 203 that is inserted and expanded within an aneurysm sac; and a coaxial catheter that is inserted into the aneurysm sac; wherein the expandable mesh is inserted through the coaxial catheter into the aneurysm sac; wherein the coaxial catheter further comprises an inner catheter 202 with a radially-symmetric distal end and an outer catheter 201 with a radially-asymmetric distal end; wherein the inner catheter and the outer catheter are nested, concentric, and/or coaxial relative to each other; wherein the coaxial catheter has a first configuration in which the inner catheter extends farther in a distal direction than the outer catheter; wherein the coaxial catheter has a second configuration in which the outer catheter extends farther in a distal direction than inner catheter; and wherein the outer catheter directs (e.g. steers, compels, or pushes) the expandable mesh out of the catheter in a selected direction, at a selected angle, and/or with a selected orientation when the coaxial catheter is in the second configuration.

The left portion of FIG. 2 shows this device at a first time when the coaxial catheter is in the first configuration. In this first configuration, the radially-symmetric inner catheter extends out the farthest and the expandable mesh extends straight out from the distal end of the catheter. The right portion of FIG. 2 shows this device at a second time when the coaxial catheter is in the second configuration. In this second configuration, the radially-asymmetric outer catheter extends out the farthest and the expandable mesh extends out from the distal end of the catheter at an angle.

In an example, the coaxial catheter can be changed between the first configuration and the second configuration by sliding (e.g. extending) the outer catheter outward (e.g. distally) relative to the inner catheter. In an example, the coaxial catheter can be changed between the first configuration and the second configuration by sliding (e.g. extending) the inner catheter inward (e.g. proximally) relative to the outer catheter. In an example, the outer catheter and/or the inner catheter can be slid remotely by a device operator. This allows a device operator to steer the expandable mesh out of the catheter in different directions, at different angles, and/or with different orientations. In a variation on the device shown in FIG. 2, the outer catheter can be the component with a radially-asymmetric distal end and the inner catheter can be the component with a radially-symmetric distal end. This this variation, the functional roles of the two configurations are switched.

In an example, the selected direction in which a mesh extends outward may not be colinear with a virtual extension of the central longitudinal axis of the coaxial catheter. In an example, the distal end of the coaxial catheter can direct (e.g. steer, compel, or push) the central axis of the expandable mesh along a vector which is not colinear with a virtual extension of the central longitudinal axis of the coaxial catheter. In an example, a device operator can change the selected direction, angle, and/or orientation of the expandable mesh by sliding, extending, and/or rotating the outer catheter. In an example, a device operator can change the selected direction, angle, and/or orientation of the central axis of the expandable mesh by sliding, extending, and/or rotating the outer catheter.

In an example, a device operator can change the selected direction, angle, and/or orientation of an expandable mesh by sliding, extending, and/or rotating the outer catheter. In an example, this sliding, extending, and/or rotating can be done by the device operator from outside a person's body. In an example, a distal end of an outer catheter can direct (e.g. steer, compel, or push) an expandable mesh to the right or left, or up and down, depending on how the catheter is rotated by the device operator. In an example, an outer catheter with a radially-asymmetric distal end portion can be rotated as well as slid in a proximal-to-distal manner. With these options, a device operator can select to have the expandable mesh either exit straight out of the coaxial catheter or in a selected direction, at a selected angle, or with a selected orientation.

In an example, the distal end of the outer catheter can be angled. In an example, the distal end can be formed by cutting across the outer catheter at an acute angle relative to the longitudinal axis of the outer catheter. In an example, the plane of the end circumference of the distal end can intersect the central longitudinal axis of the outer catheter at an acute angle. In an example, this angle can be between 25 and 75 degrees. In an example, the distal end of the outer catheter can be tilted an acute angle. In an example, the distal end of the outer catheter can be tilted at an angle of 25 to 75 degrees relative to a central longitudinal axis of the outer catheter. In an example, the distal end of the outer catheter can be curved to form an acute angle (like a piece of plumbing pipe).

In an example, an expandable mesh can expand within an aneurysm sac into a bowl, cup, hemispherical, or hemi-elliptical shape. In an example, an expandable mesh can expand within an aneurysm sac into a shape which is section (e.g. the lower half) of a torus. In an example, an expandable mesh can expand within an aneurysm sac into a globular, spherical, or elliptical shape. In an example, an expandable mesh can expand within an aneurysm sac into a funnel or parabolic shape. In an example, an expandable mesh can expand within an aneurysm sac into a hyperbolic or hourglass shape. In an example, the direction and/or vector of a central axis of an expandable mesh can be changed by sliding, extending, and/or rotating an outer catheter with a radially-asymmetric distal end. Relevant variations discussed elsewhere in this disclosure or in priority-linked disclosures can also be applied to this example.

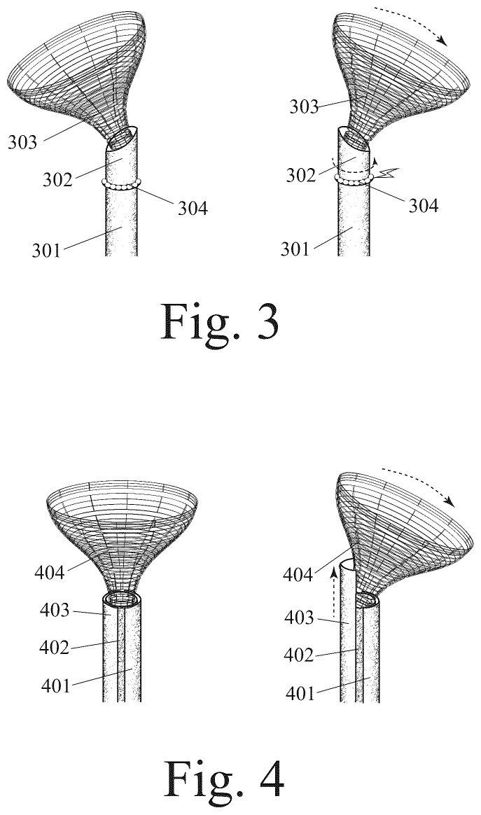

FIG. 3 shows two views, at two different times, of an intrasaccular aneurysm occlusion device comprising: an expandable mesh (e.g. mesh, lattice, braid, stent, and/or neck bridge) 303 that is inserted and expanded within an aneurysm sac; and a catheter 301 that is inserted into the aneurysm sac, wherein the expandable mesh is inserted through the catheter into the aneurysm sac, wherein the catheter further comprises a radially-asymmetric distal end portion 302 and a rotational joint (e.g. ball bearing) 304, and wherein the rotational joint is between the distal end portion and the rest of the catheter.

In an example, the radially-asymmetric distal portion directs (e.g. steers, compels, and/or pushes) the expandable mesh out of the catheter in a selected direction, at a selected angle, and/or with a selected orientation. In an example, the device operator can change the selected direction, angle, and/or orientation by which the expandable mesh exits the catheter by rotating the distal end portion around the rotational joint relative to the rest of the catheter. In an example, the device can further comprise an (electromagnetic) actuator which the device operator controls to rotate the joint.

The left portion of FIG. 3 shows this device at a first time when the distal end portion of the catheter is rotated in a first configuration via the rotational joint, causing the expandable mesh to extend out from the catheter in a first direction, at a first angle, and/or with a first orientation. The right portion of FIG. 3 shows this device at a second time when the distal end portion of the catheter is rotated in a second configuration via the rotational joint, causing the expandable mesh to extend out from the catheter in a second direction, at a second angle, and/or with a second orientation.

In an example, the device can further comprise an electrical motor (e.g. micromotor) which rotates the distal end portion of the catheter, wherein the device operator controls this motor remotely from outside a person's body. In an example, the device can further comprise an electroconductive pathway (e.g. wire) along the catheter which transmits power and/or operational commands from a proximal location (e.g. outside a person's body) to the electrical motor in order to control rotation of the rotational joint. In an example, the rotational joint can further comprise a ball bearing.

In an example, a distal end portion of a catheter can be angled. In an example, a distal end portion can be formed by cutting across the catheter at an acute angle between 25 and 75 degrees. In an example, a circumference of a distal end portion can be tilted an acute angle between 25 and 75 degrees. In an example, the end of the distal end portion can be curved to form an acute angle (like a piece of plumbing pipe). In an example, the distal end portion of the catheter which is rotated by the rotational joint can be between 2 and 10 mm long. In an example, the distal end portion of the catheter which is rotated by the rotational joint can be between 5 and 20 mm long.

In an example, a side of the distal end portion can direct (e.g. steer, compel, and/or push) an expandable mesh in a selected direction, at a selected angle, and/or with a selected orientation as the mesh exits a catheter. In an example, a first side of the circumference of the distal end portion can extend out farther than a second slide of the circumference, wherein contact between the first side and an expandable mesh directs (e.g. steers, compels, and/or pushes) the expandable mesh out of the catheter in a selected direction, at a selected angle, and/or with a selected orientation. In an example, one side of the distal end portion can direct (e.g. steer and/or push) an expandable mesh to the right or left, or up or down, as the mesh exits the catheter.

In an example, an expandable mesh can expand within an aneurysm sac into a bowl, cup, hemispherical, or hemi-elliptical shape. In an example, an expandable mesh can expand within an aneurysm sac into a shape which is section (e.g. the lower half) of a torus. In an example, an expandable mesh can expand within an aneurysm sac into a globular, spherical, or elliptical shape. In an example, an expandable mesh can expand within an aneurysm sac into a funnel or parabolic shape. In an example, an expandable mesh can expand within an aneurysm sac into a hyperbolic or hourglass shape. In an example, the direction and/or vector of a central axis of an expandable mesh can be changed by rotating a radially-asymmetric distal end portion of a catheter via a rotational joint. Relevant variations discussed elsewhere in this disclosure or in priority-linked disclosures can also be applied to this example.

FIG. 4 shows two views, at two different times, of an intrasaccular aneurysm occlusion device comprising: an expandable mesh (e.g. mesh, lattice, braid, stent, and/or neck bridge) 404 that is inserted and expanded within an aneurysm sac; and a multi-section catheter that is inserted into the aneurysm sac; wherein the expandable mesh is inserted through the multi-section catheter into the aneurysm sac; wherein the multi-section catheter further comprises a first-side outer section 403, a second-side outer section 401, and an inner catheter 402; wherein the multi-section catheter has a first configuration in which the first-side outer section and the second-side outer section extend in a distal direction the same distance; wherein the multi-section catheter has a second configuration in which the first-side outer section extends farther in a distal direction than the second-side outer section; and wherein the multi-section catheter has a third configuration in which the second-side outer section extends farther in a distal direction than the first-side outer section. In an example, the multi-section catheter is changed between the first, second, and third configurations by sliding the first-side outer section and/or the second-side outer section in a proximal-to-distal direction, or vice versa.

The left portion of FIG. 4 shows this device at a first time when the multi-section catheter is in the first configuration, wherein the first-side and second-side outer sections extend equally in a distal direction. In this first configuration, a central axis of the expandable mesh is colinear with a virtual extension of the central longitudinal axis of the multi-section catheter. The right portion of FIG. 4 shows this device at a second time when the multi-section catheter is in the second configuration, wherein the first-side outer section extends farther in a distal direction than the second-side outer section. In this second configuration, the central axis of the expandable mesh is steered to the right of the central longitudinal axis of the multi-section catheter.

In an example, the device operator can steer the expandable mesh in different directions, at different angles, and/or with different orientations by sliding (in a proximal-to-distal manner, or vice versa) the first-side outer section and/or the second-side outer section relative to each other. In an example, a first-side outer section and/or a second-side outer section can span between 20% and 50% of the circumference of the inner catheter. In an example, the first-side outer section and/or the second-side outer section can slide along a longitudinal channel or track on the outer surface of the inner catheter. In an example, the device operator can slide the first-outer section and/or the second outer section remotely, from the proximal end of the catheter outside a person's body.

In an example, an expandable mesh can expand within an aneurysm sac into a bowl, cup, hemispherical, or hemi-elliptical shape. In an example, an expandable mesh can expand within an aneurysm sac into a shape which is section (e.g. the lower half) of a torus. In an example, an expandable mesh can expand within an aneurysm sac into a globular, spherical, or elliptical shape. In an example, an expandable mesh can expand within an aneurysm sac into a funnel or parabolic shape. In an example, an expandable mesh can expand within an aneurysm sac into a hyperbolic or hourglass shape. Relevant variations discussed elsewhere in this disclosure or in priority-linked disclosures can also be applied to this example.

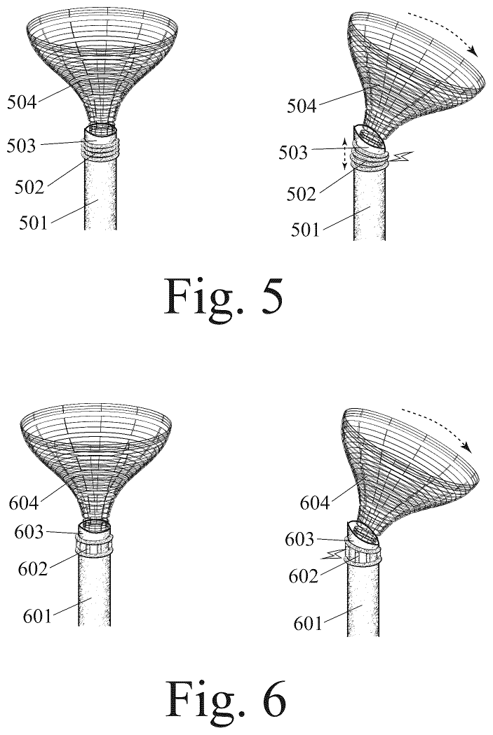

FIG. 5 shows two views, at two different times, of an intrasaccular aneurysm occlusion device comprising: an expandable mesh (e.g. mesh, lattice, braid, stent, and/or neck bridge) 504 that is inserted and expanded within an aneurysm sac; and a catheter 501 that is inserted into the aneurysm sac, wherein the expandable mesh is inserted through the catheter into the aneurysm sac, wherein the catheter further comprises a movable (e.g. pivoting and/or rotating) distal end portion 503, and wherein the catheter further comprises a joint 502 between the distal end portion and the rest of the catheter.

The left portion of FIG. 5 shows this device at a first time when the distal end portion of the catheter is longitudinally-aligned with the rest of the catheter and the expandable mesh extends straight out from the catheter. The right portion of FIG. 5 shows this device at a second time when the distal end portion of the catheter is moved (e.g. pivoted and/or rotated) by movement of the joint and the expandable mesh extends out from the catheter in a selected direction, at a selected angle, or with a selected orientation.

In an example, the joint can be a pivoting joint. In an example, the joint can be a rotating joint. In an example, the joint can be articulated and/or pleated. In an example, the device can further comprise an electromagnetic motor which moves the joint, wherein the device operator can remotely control the electromagnetic motor to selectively move the joint (and, thus, the distal end portion of the catheter). In an example, the pivoting distal portion can directs (e.g. steer, compel, and/or push) the expandable mesh in a selected direction, at a selected angle, and/or with a selected orientation as the mesh exits the catheter. In an example, a joint can have a first configuration with a generally cylindrical shape and a second configuration with a wedge shape. In an example, a joint can be changed from the first configuration to the second configuration, or vice versa, by activation of an actuator by the device operator.

In an example, a device operator can change the selected direction, angle, and/or orientation of the expandable mesh by activating the joint to move the distal end portion of the catheter. In an example, the device can further comprise an actuator which moves (e.g. pivots and/or rotates) the joint. In an example, the actuator can be an electromagnetic actuator (e.g. electric motor). In an example, a device operator can control this actuator remotely from outside the person's body. In an example, a device operator can steer (e.g. steer, compel, and/or push) the expandable mesh to the right or left, or up or down, by operating the actuator which moves the joint. In another example, an actuator can be a piezoelectric actuator. In another example, an actuator can be a hydraulic or pneumatic actuator.

In an example, an expandable mesh can expand within an aneurysm sac into a bowl, cup, hemispherical, or hemi-elliptical shape. In an example, an expandable mesh can expand within an aneurysm sac into a shape which is section (e.g. the lower half) of a torus. In an example, an expandable mesh can expand within an aneurysm sac into a globular, spherical, or elliptical shape. In an example, an expandable mesh can expand within an aneurysm sac into a funnel or parabolic shape. In an example, an expandable mesh can expand within an aneurysm sac into a hyperbolic or hourglass shape. Relevant variations discussed elsewhere in this disclosure or in priority-linked disclosures can also be applied to this example.

FIG. 6 shows two views, at two different times, of an intrasaccular aneurysm occlusion device comprising: an expandable mesh (e.g. mesh, lattice, braid, stent, and/or neck bridge) 604 that is inserted and expanded within an aneurysm sac; and a catheter 601 that is inserted into the aneurysm sac, wherein the expandable mesh is inserted through the catheter into the aneurysm sac, wherein the catheter further comprises a movable (e.g. pivoting and/or rotating) distal end portion 603, and wherein the catheter further comprises an annular array of actuators (e.g. solenoids, pistons, and/or MEMS devices) 602 between the distal end portion and the rest of the catheter.

The left portion of FIG. 6 shows this device at a first time when the distal end portion of the catheter is longitudinally-aligned with the rest of the catheter and the expandable mesh extends straight out from the catheter. The right portion of FIG. 6 shows this device at a second time when the distal end portion of the catheter is moved (e.g. pivoted and/or rotated) by activation of the annular array of actuators (e.g. solenoids and/or pistons) and the expandable mesh extends out from the catheter in a selected direction, at a selected angle, or with a selected orientation.

In an example, an annular array of actuators can comprise an annular array of solenoids which are controlled remotely by a person operating the device. In an example, selective extension or contract of solenoids on a selected side of the catheter can pivot, tilt, and/or rotate the distal end portion of the catheter, thereby changing the direction, angle, and/or orientation of the expandable mesh as it exits the catheter. In an example, the device can further comprise one or more electroconductive pathways (e.g. microwires) along the catheter which transmit electrical power and/or operating commands from the device operator to the annular array. In an example, an annular array can comprise three or more solenoids which are equally distributed around the circumference of the catheter. In an example, an annular array can comprise four or six solenoids which are equally distributed around the circumference of the catheter.

In an example, an annular array of actuators can comprise an annular array of (hydraulic) pistons which are controlled remotely by a person operating the device. In an example, selective extension or contract of (hydraulic) pistons on a selected side of the catheter can pivot, tilt, and/or rotate the distal end portion of the catheter, thereby changing the direction, angle, and/or orientation of the expandable mesh as it exits the catheter. In an example, the device can further comprise one or more microfluidic lumens along the catheter which convey a flowable substance (e.g. fluid, gas, or gel) to the pistons. In an example, an annular array can comprise three or more (hydraulic) pistons which are equally distributed around the circumference of the catheter. In an example, an annular array can comprise four or six (hydraulic) pistons which are equally distributed around the circumference of the catheter.

In an example, an expandable mesh can expand within an aneurysm sac into a bowl, cup, hemispherical, or hemi-elliptical shape. In an example, an expandable mesh can expand within an aneurysm sac into a shape which is section (e.g. the lower half) of a torus. In an example, an expandable mesh can expand within an aneurysm sac into a globular, spherical, or elliptical shape. In an example, an expandable mesh can expand within an aneurysm sac into a funnel or parabolic shape. In an example, an expandable mesh can expand within an aneurysm sac into a hyperbolic or hourglass shape. Relevant variations discussed elsewhere in this disclosure or in priority-linked disclosures can also be applied to this example.

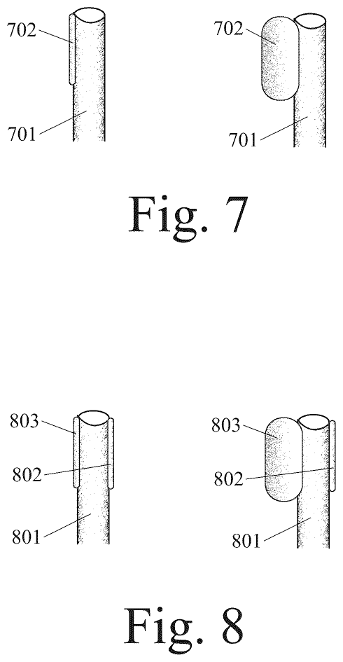

FIG. 7 shows two views, at two different times, of an intrasaccular aneurysm occlusion device comprising: a catheter 701 that delivers a mesh (e.g. mesh, lattice, braid, stent, and/or neck bridge) into an aneurysm sac; and an expandable component (e.g. fluid-filled balloon) 702 on one side of the outer surface of a distal end portion of the catheter. When the expandable component is expanded, this component comes into contact with an aneurysm wall. This contact changes the direction, angle, and/or orientation the distal end portion of the catheter which, in turn, changes the direction, angle, and/or orientation by which the mesh exits the catheter.

The left portion of FIG. 7 shows this device at a first time, before the expandable component is expanded. The right portion of FIG. 7 shows this device at a second time, after the expandable component has been expanded. In an example, a device operator can steer an expandable mesh exiting the catheter in different directions, at different angles, and/or with different orientations by changing the extent to which the expandable component is expanded. In an example, an expandable component can have an oblong (longitudinal cross-sectional) shape.

In an example, an expandable component can be a balloon. In an example, an expandable component can be expanded by being filled with a flowable substance. In an example, a flowable substance can be a liquid. In an example, a flowable substance can be a gas. In an example, the device can further comprise a flowable substance conduit and/or channel through which the flowable substance is sent into the expandable component. Relevant variations discussed elsewhere in this disclosure or in priority-linked disclosures can also be applied to this example.

FIG. 8 shows two views, at two different times, of an intrasaccular aneurysm occlusion device comprising: a catheter 801 that delivers a mesh (e.g. mesh, lattice, braid, stent, and/or neck bridge) into an aneurysm sac; and a plurality of expandable components (e.g. fluid-filled balloons) 802 and 803 at different circumferential locations on the outer surface of a distal end portion of the catheter. When one or more of the expandable components are expanded, they can come into contact with an aneurysm wall. This contact can change the direction, angle, and/or orientation the distal end portion of the catheter which, in turn, changes the direction, angle, and/or orientation by which the mesh exits the catheter.

The left portion of FIG. 8 shows this device at a first time, before one or more of the expandable components have been expanded. The right portion of FIG. 8 shows this device at a second time, after one or more of the expandable components have been expanded. In an example, a device operator can steer an expandable mesh exiting the catheter in different directions, at different angles, and/or with different orientations by changing the extent to which one or more of the expandable components are expanded.

In an example, a plurality of expandable components can be distributed equidistantly around the circumference of the distal end portion of the catheter. In an example, there can be two expandable components, on opposite sides respectively, of the distal end portion. In an example, there can be four or more expandable components which are distributed equidistantly around the circumference of the distal end portion. In an example, an expandable component can have an oblong (longitudinal cross-sectional) shape.

In an example, an expandable component can be a balloon. In an example, an expandable component can be expanded by being filled with a flowable substance. In an example, a flowable substance can be a liquid. In an example, a flowable substance can be a gas. In an example, the device can further comprise a conduit and/or channel on the catheter through which a flowable substance is sent into the expandable component. In an example, a device operator can control the flow of a flowable substance through one or more conduits or channels into one or more expandable components in order to select the direction, angle, and/or orientation by which a mesh exits the catheter. Relevant variations discussed elsewhere in this disclosure or in priority-linked disclosures can also be applied to this example.

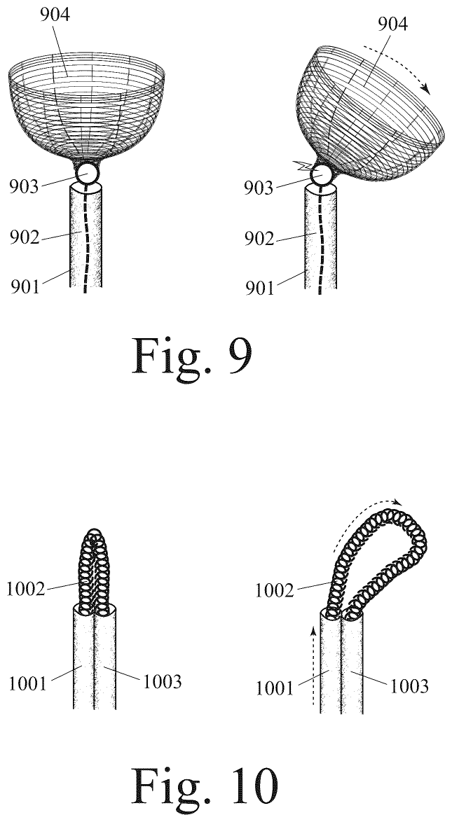

FIG. 9 shows two views, at two different times, of an intrasaccular aneurysm occlusion device comprising: an expandable mesh (e.g. mesh, lattice, braid, stent, and/or neck bridge) 904 that is inserted and expanded within an aneurysm sac; a catheter 901 that is inserted into the aneurysm sac, wherein the expandable mesh delivered through the catheter into the aneurysm sac; a movable (e.g. pivoting or rotating) joint 903 between the expandable mesh and the catheter; and a wire 902 within the catheter.

The left portion of FIG. 9 shows this device at a first time when the movable (e.g. pivoting or rotating) joint is aligned with the central longitudinal axis of the catheter and the expandable mesh extends straight out from the end of catheter. The right portion of FIG. 9 shows this device at a second time when the movable (e.g. pivoting or rotating) joint is tilted and/or rotated and the expandable mesh extends out from the end of catheter at an acute angle relative to the central longitudinal axis of the catheter. In an example, a device operator can change the direction, angle, and/or orientation of the expandable mesh by selectively moving (e.g. pivoting and/or rotating) the movable joint.

In an example, the movable (e.g. pivoting and/or rotating) joint can be connected to the wire, wherein the joint is moved (e.g. pivoted and/or rotated) by pushing, pulling, or rotating the wire. In an example, the movable (e.g. pivoting and/or rotating) joint can be connected to the wire, wherein the joint is moved (e.g. pivoted and/or rotated) when the device operator pushes, pulls, or rotates the wire. In an example, the device can further comprise an electromagnetic actuator (e.g. electrical micromotor) which moves (e.g. pivots and/or rotates) the movable joint. In an example, the wire can transmit electrical power and/or commands to such an electromagnetic actuator. In an example, the device operator can control the actuator remotely by transmitting commands through the wire. In an example, a device operator can change the selected direction, angle, and/or orientation of the expandable mesh by moving (e.g. pivoting and/or rotating) the movable joint. In an example, a device operator can steer (e.g. steer, compel, and/or push) the expandable mesh to the right or left, or up or down, by operating an actuator which moves the movable joint.

In an example, an expandable mesh can expand within an aneurysm sac into a bowl, cup, hemispherical, or hemi-elliptical shape. In an example, an expandable mesh can expand within an aneurysm sac into a shape which is section (e.g. the lower half) of a torus. In an example, an expandable mesh can expand within an aneurysm sac into a globular, spherical, or elliptical shape. In an example, an expandable mesh can expand within an aneurysm sac into a funnel or parabolic shape. In an example, an expandable mesh can expand within an aneurysm sac into a hyperbolic or hourglass shape. Relevant variations discussed elsewhere in this disclosure or in priority-linked disclosures can also be applied to this example.

FIG. 10 shows two views, at two different times, of an intrasaccular aneurysm occlusion device comprising: a dual catheter that is inserted into an aneurysm sac, wherein the dual catheter further comprises a first catheter 1001 and a second catheter 1002, and wherein the first catheter and the second catheter are parallel to each other; and a coil loop 1002 which is inserted through the dual catheter into the aneurysm sac, wherein a first branch of the coil loop is inserted into the aneurysm sac through the first catheter and a second branch of the coil loop is inserted into the aneurysm sac through the second catheter.

In an example, a device operator can selectively steer the coil loop in different directions by non-uniform insertion of the first branch and the second branch (e.g. by inserting one of the branches more than the other branch). The left portion of FIG. 10 shows this device at a first time when the two branches are uniformly inserted and the coil loop extends straight out from the dual catheter. The right portion of FIG. 10 shows this device at a second time when the two branches are non-uniformly inserted, which steers the coil loop to the right. Relevant variations discussed elsewhere in this disclosure or in priority-linked disclosures can also be applied to this example.

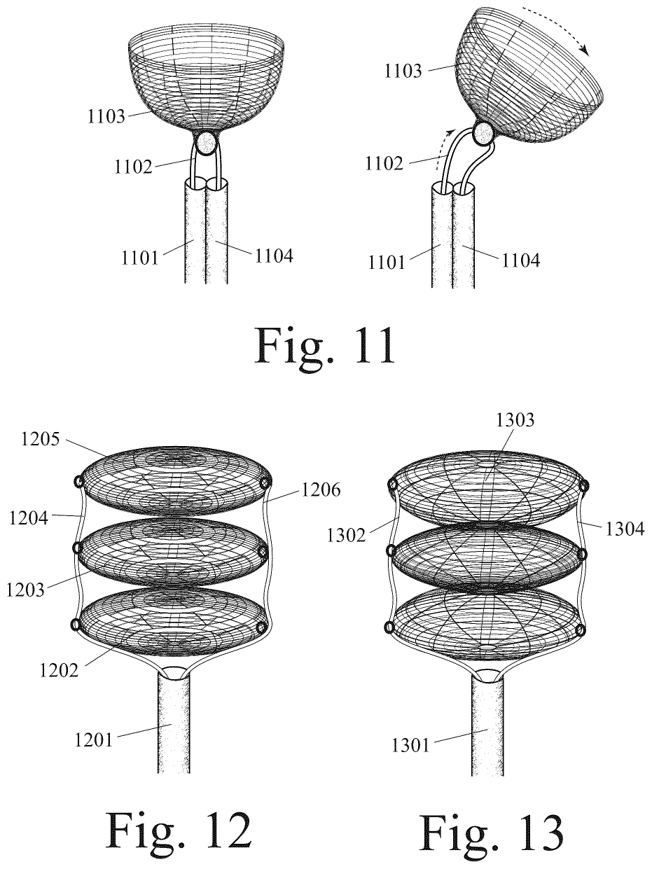

FIG. 11 shows two views, at two different times, of an intrasaccular aneurysm occlusion device comprising: a dual catheter that is inserted into an aneurysm sac, wherein the dual catheter further comprises a first catheter 1101 and a second catheter 1104, and wherein the first catheter and the second catheter are parallel to each other; a coil loop 1102 which is inserted through the dual catheter into the aneurysm sac, wherein a first branch of the coil loop is inserted into the aneurysm sac through the first catheter and a second branch of the coil loop is inserted into the aneurysm sac through the second catheter; and an expandable mesh (e.g. mesh, lattice, braid, stent, and/or neck bridge) 1103 that is inserted and expanded within an aneurysm sac, wherein the mesh is attached to the coil loop.

In an example, a device operator can selectively steer the expandable mesh in different directions, at different angles, and/or with different orientations by non-uniform insertion of the first branch and the second branch into an aneurysm sac (e.g. by inserting one of the branches a farther distance than the other branch). The left portion of FIG. 11 shows this device at a first time when the two branches are uniformly inserted and the expandable mesh extends straight out from the dual catheter. The right portion of FIG. 11 shows this device at a second time when the two branches are non-uniformly inserted, which steers the expandable mesh to the right. Relevant variations discussed elsewhere in this disclosure or in priority-linked disclosures can also be applied to this example.

FIG. 12 shows an intrasaccular aneurysm occlusion device comprising: a catheter 1201 that is inserted into an aneurysm sac; a longitudinal (e.g. proximal-to-distal) series of expandable meshes (1202, 1203, and 1205) which are inserted through the catheter into the aneurysm sac; a first flexible longitudinal member (e.g. wire, suture, band, or string) 1204 which connects expandable meshes in the longitudinal series; and a second flexible longitudinal member (e.g. wire, suture, band, or string) 1206 which connects expandable meshes in the longitudinal series.

In an example, a device operator can selectively steer the longitudinal series of expandable meshes (e.g. meshes, lattices, braids, stents, and/or neck bridges) in different directions, at different angles, and/or with different orientations by differentially pulling and/or pushing the first and second longitudinal members. In an example, a device operator can selectively steer the longitudinal series of expandable meshes left or right, or up or down, by differentially pulling and/or pushing the first and second longitudinal members.

In an example, a device operator can pull and/or push the first and second longitudinal members from a location outside a person's body (e.g. the person who has the aneurysm). In an example, a device operator can manually pull or push the first and/or second longitudinal members to steer the longitudinal series of meshes in a selected direction, at a selected angle, or with a selected orientation. In an example, a device operator can control one or more (electromagnetic) actuators which pull or push the first and/or second longitudinal members in order to steer the longitudinal series of meshes.

In an example, a device operator can selectively steer the longitudinal series of expandable meshes in different directions, at different angles, and/or with different orientations by pulling and/or pushing the first longitudinal member differently than the second longitudinal member. In an example, a device operator can selectively steer the longitudinal series of expandable meshes left or right, or up or down, by pulling and/or pushing the first longitudinal member to a greater extent than the second longitudinal member. In an example, a device operator can selectively steer the longitudinal series of expandable meshes left or right, or up or down, by pulling and/or pushing the first and second longitudinal members by different amounts.

In the example shown in FIG. 12, pushing the first longitudinal member and/or pulling the second longitudinal member would steer the longitudinal series of expandable meshes to the right. In the example shown in FIG. 12, pulling the first longitudinal member and/or pushing the second longitudinal member would steer the longitudinal series of expandable meshes to the left.

In an example, a longitudinal member can be a wire. In an example, a longitudinal member can be a suture, thread, or string. In an example, a longitudinal member can be an elastic band. In an example, a longitudinal member can be a chain. In an example, a longitudinal member can be piezoelectric. In this example, a device comprises two longitudinal members which connect expandable meshes, with one longitudinal member on each side (e.g. on opposite sides) of the expandable meshes. In another example, a device can comprise a plurality of three or more longitudinal members which connect expandable meshes, wherein connection locations between the plurality of longitudinal members and an expandable mesh are equally (e.g. equidistantly) distributed around the circumference of the expandable mesh.

In an example, a longitudinal member can be attached to each of the expandable meshes in the longitudinal series of expandable meshes. In an example, a first longitudinal member can be attached to a first side of one or more expandable meshes and a second longitudinal member can be attached to the opposite side of the one or more expandable mesh.

In an example, a connection between a longitudinal member and an expandable mesh can be fixed (e.g. not sliding). In another example, a connection between a longitudinal member and an expandable mesh can be a sliding connection (e.g. a loop or hole through which the longitudinal member slides). In an example, connections with some of the expandable meshes can be fixed and connections with other expandable meshes can be slidable. In an example, connections between a longitudinal flexible member and the most distal mesh in the series can be fixed connections and connections between the longitudinal flexible member and the rest of the meshes in the series can be sliding connections.

In an example, a device can comprise three longitudinal members (e.g. wires, sutures, bands, or strings): (a) a central longitudinal member which connects the centers of expandable meshes in a longitudinal series; (b) a first-side longitudinal member which connects first sides of expandable meshes in a longitudinal series; and (c) a second-side longitudinal member which connects second (e.g. opposite) sides of expandable meshes in the longitudinal series.

In an example, a device can comprise three longitudinal members (e.g. wires, sutures, bands, or strings): (a) a central longitudinal member which connects the centers of expandable meshes in a longitudinal series; (b) a first non-central longitudinal member which connects first non-central locations on expandable meshes in a longitudinal series; and (c) a second non-central longitudinal member which connects second non-central locations on expandable meshes in the longitudinal series.

In an example, all of the expandable meshes in a series can have convex shapes. In an example, an expandable mesh can have an ellipsoid, ovaloid, and/or oblate spheroid shape. In an example, an expandable mesh can have a toroidal shape. In an example, an expandable mesh can have an polyhedron shape. In another example, one of the expandable meshes in the longitudinal series can be convex and other expandable meshes in the longitudinal series can be concave. In another example, all of the expandable meshes can have concave shapes. In an example, two or more of the expandable meshes can be nested after their deployment in the aneurysm sac.

In an example, an expandable mesh can self-expand within an aneurysm sac after it exits a catheter. In an example, an expandable mesh can comprise a mesh, lattice, braid, stent, and/or neck bridge. In this example, expandable meshes in the series are separately-created components which are connected to each other by the first and second longitudinal members. In this example, the longitudinal series comprises three expandable meshes. In another example, a longitudinal series can comprise four or more expandable meshes. In this example, the expandable meshes are centrally aligned when they extend straight out of the catheter. In this example, the centroids of the expandable meshes are colinear when they extend straight out of the catheter.

In an example, expandable meshes in the longitudinal series can all be the same size. In another example, expandable meshes which are more distal can be smaller than expandable meshes which are more proximal in the longitudinal series. In another example, expandable meshes which are more distal can be larger than expandable meshes which are more proximal in the longitudinal series. In an example, the size of expandable meshes in a series can (progressively) decrease with increased distance from an aneurysm neck. In an example, expandable meshes in the longitudinal series can all have the same shape. In another example, expandable meshes which are more distal can have different shapes than expandable meshes which are more proximal. In another example, expandable meshes which are more distal can be more convex than expandable meshes which are more proximal.

In an example, expandable meshes in the longitudinal series can all have the same elasticity level and/or Shore value. In another example, expandable meshes which are more distal can have greater elasticity levels and/or lower Shore values. In an example, expandable meshes in the longitudinal series can all have the same porosity level. In another example, expandable meshes which are more distal can have greater porosity levels. In an example, expandable meshes in the longitudinal series can all have the same mesh density. In another example, expandable meshes which are more distal can have lower mesh densities. Relevant variations discussed elsewhere in this disclosure or in priority-linked disclosures can also be applied to this example.

FIG. 13 shows an intrasaccular aneurysm occlusion device comprising: a catheter 1301 that is inserted into an aneurysm sac; a longitudinally undulating and/or multi-lobed mesh (e.g. mesh, lattice, braid, stent, and/or neck bridge) 1303 which is inserted through the catheter into the aneurysm sac; a first flexible longitudinal member (e.g. wire, suture, band, or string) 1302 attached to (a first side of) the undulating and/or multi-lobed expandable mesh; and a second flexible longitudinal member (e.g. wire, suture, band, or string) 1304 attached to (a second side of) the undulating and/or multi-lobed expandable mesh.

In an example, a device operator can selectively steer the longitudinally undulating and/or multi-lobed mesh (e.g. mesh, lattice, braid, stent, and/or neck bridge) in different directions, at different angles, and/or with different orientations by differentially pulling and/or pushing the first and second longitudinal members. In an example, a device operator can selectively steer the longitudinally undulating and/or multi-lobed mesh left or right, or up or down, by differentially pulling and/or pushing the first and second longitudinal members.

In an example, a device operator can pull and/or push the first and second longitudinal members from a location outside a person's body (e.g. the person who has the aneurysm). In an example, a device operator can manually pull or push the first and/or second longitudinal members to steer the longitudinally undulating and/or multi-lobed mesh in a selected direction, at a selected angle, or with a selected orientation. In an example, a device operator can control one or more (electromagnetic) actuators which pull or push the first and/or second longitudinal members in order to steer the longitudinally undulating and/or multi-lobed mesh.

In an example, a device operator can selectively steer the longitudinally undulating and/or multi-lobed mesh in different directions, at different angles, and/or with different orientations by pulling and/or pushing the first longitudinal member differently than the second longitudinal member. In an example, a device operator can selectively steer the longitudinally undulating and/or multi-lobed mesh left or right, or up or down, by pulling and/or pushing the first longitudinal member to a greater extent than the second longitudinal member. In an example, a device operator can selectively steer the longitudinally undulating and/or multi-lobed mesh left or right, or up or down, by pulling and/or pushing the first and second longitudinal members by different amounts.

In the example shown in FIG. 13, pushing the first longitudinal member and/or pulling the second longitudinal member would steer the longitudinally undulating and/or multi-lobed mesh to the right. In the example shown in FIG. 13, pulling the first longitudinal member and/or pushing the second longitudinal member would steer the longitudinally undulating and/or multi-lobed mesh to the left.

In an example, a longitudinal member can be a wire. In an example, a longitudinal member can be a suture, thread, or string. In an example, a longitudinal member can be an elastic band. In an example, a longitudinal member can be a chain. In an example, a longitudinal member can be piezoelectric. In this example, a device comprises two longitudinal members which are attached to opposite sides of the longitudinally undulating and/or multi-lobed mesh. In another example, a device can comprise a plurality of three or more longitudinal members, wherein connection locations between the plurality of longitudinal members and the longitudinally undulating and/or multi-lobed mesh are equally (e.g. equidistantly) distributed around the circumference of the mesh.

In an example, undulations and/or lobes in a longitudinally undulating and/or multi-lobed mesh can all be the same size, width, and/or diameter. In another example, distal undulations and/or lobes can be smaller than proximal undulating and/or lobes in a longitudinally undulating and/or multi-lobed mesh. In an example, undulations and/or lobes in a longitudinally undulating and/or multi-lobed mesh can be sinusoidal.

In an example, there can be three or more undulations and/or lobes in a longitudinally undulating and/or multi-lobed mesh. In an example, there can be three or more undulations and/or lobes in a longitudinally undulating and/or multi-lobed mesh: a proximal undulation and/or lobe; a middle undulation and/or lobe; and a distal undulation and/or lobe. In an example, undulations and/or lobes in a longitudinally undulating and/or multi-lobed mesh can be coaxial. In an example, the central axes of undulations and/or lobes in a longitudinally undulating and/or multi-lobed mesh can be colinear. In an example, a longitudinally undulating and/or multi-lobed mesh can self-expand within an aneurysm sac after it exits a catheter.

In an example, lobes in a longitudinally undulating and/or multi-lobed mesh can all have the same elasticity level and/or Shore value. In another example, lobes which are more distal can have greater elasticity levels and/or lower Shore values. In an example, lobes in a longitudinally undulating and/or multi-lobed mesh can all have the same porosity level. In another example, lobes which are more distal can have greater porosity levels. In an example, lobes in a longitudinally undulating and/or multi-lobed mesh can all have the same mesh density. In another example, lobes which are more distal can have lower mesh densities. Relevant variations discussed elsewhere in this disclosure or in priority-linked disclosures can also be applied to this example.

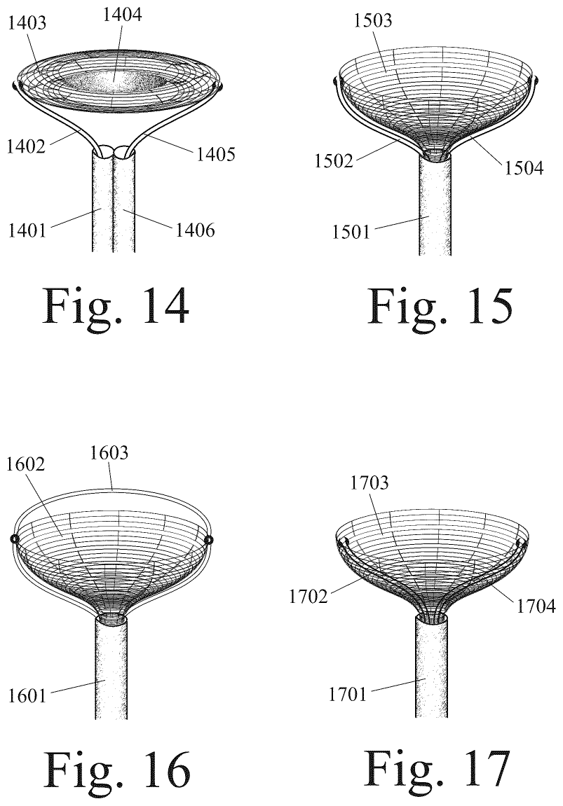

FIG. 14 shows an intrasaccular aneurysm occlusion device comprising: a dual catheter that is inserted into an aneurysm sac, wherein the dual catheter further comprises a first catheter 1401 and a second catheter 1406, and wherein the first catheter and the second catheter are parallel to each other; a first flexible longitudinal member (e.g. wire, suture, band, or string) 1402 which is inserted into the aneurysm sac through the first catheter; a second flexible longitudinal member (e.g. wire, suture, band, or string) 1405 which is inserted into the aneurysm sac through the second catheter; and a toroidal mesh (e.g. mesh, lattice, braid, stent, and/or neck bridge) 1403 that is inserted and expanded within the aneurysm sac, wherein the toroidal mesh is attached to the first flexible longitudinal member and to the second flexible longitudinal member. In an example, the toroidal mesh can self-expand within the aneurysm sac after it exits the catheter.

In this example, the device further comprises a low-porosity (e.g. fluid-impermeable) component 1404 which spans the central opening of the toroidal mesh. In another example in which there is no low-porosity component spanning the central opening of the toroidal mesh, embolic members and/or material (e.g. embolic coils, beads, hydrogels, or congealing liquid) can be inserted through the central opening into the aneurysm sac to occlude the aneurysm sac more thoroughly.

In an example, a device operator can selectively steer the toroidal mesh (e.g. mesh, lattice, braid, stent, and/or neck bridge) in different directions, at different angles, and/or with different orientations by differentially pulling and/or pushing the first and second longitudinal members. In an example, a device operator can selectively steer the toroidal mesh left or right, or up or down, by differentially pulling and/or pushing the first and second longitudinal members.

FIG. 14 shows this device when the two branches extend out from the catheter by the same amounts and the expandable mesh extends straight out from the dual catheter. In this example, pushing the first longitudinal member and/or pulling the second longitudinal member would steer the toroidal mesh to the right. In this example, pulling the first longitudinal member and/or pushing the second longitudinal member would steer the toroidal mesh to the left.

In an example, a device operator can manually pull or push the first and/or second longitudinal members to steer the toroidal mesh in a selected direction, at a selected angle, or with a selected orientation. In an example, a device operator can control one or more (electromagnetic) actuators which pull or push the first and/or second longitudinal members in order to steer the toroidal mesh.

In an example, a longitudinal member can be a wire. In an example, a longitudinal member can be a suture, thread, or string. In an example, a longitudinal member can be an elastic band. In an example, a longitudinal member can be a chain. In an example, a longitudinal member can be piezoelectric. In this example, the device comprises two longitudinal members which are attached to opposite sides of the toroidal mesh. In another example, a device can comprise a plurality of three or more longitudinal members, wherein connection locations between the plurality of longitudinal members and the toroidal mesh are equally (e.g. equidistantly) distributed around the circumference of the mesh. Relevant variations discussed elsewhere in this disclosure or in priority-linked disclosures can also be applied to this example.

FIG. 15 shows an intrasaccular aneurysm occlusion device comprising: an expandable mesh (e.g. mesh, lattice, braid, stent, and/or neck bridge) 1503 that is inserted and expanded within an aneurysm sac; a catheter 1501 that is inserted into the aneurysm sac, wherein the expandable mesh is inserted through the catheter into the aneurysm sac; a first flexible longitudinal member (e.g. wire, suture, band, or string) 1502 which is attached to a first portion of the expandable mesh; and a second flexible longitudinal member (e.g. wire, suture, band, or string) 1504 which is attached to a second portion of the expandable mesh; wherein the direction, angle, and/or orientation of the expandable mesh can be changed by differentially pulling and/or pushing the first flexible longitudinal member and/or the second flexible longitudinal member.

FIG. 15 shows this device in a first configuration in which the expandable mesh extends straight out of the catheter (e.g. axially aligned with the longitudinal axis of the catheter). In an example, a device operator can subsequently steer the expandable mesh to the right or left, or up or down, by differentially pulling and/or pushing the first and/or second longitudinal members. In this example, a device operator can steer (e.g. tilt or rotate) the expandable mesh to the right by pushing the first longitudinal member and/or pulling the second longitudinal member. In this example, a device operator can steer (e.g. tilt or rotate) the expandable mesh to the left by pulling the first longitudinal member and/or pushing the second longitudinal member.

In an example, a device operator can selectively steer the expandable mesh (e.g. mesh, lattice, braid, stent, and/or neck bridge) in a selected direction, at a selected angle, and/or with a selected orientations by selectively and differentially pulling and/or pushing the first and/or second longitudinal members. In an example, a device operator can push or pull one or both longitudinal members manually from a location outside a person's body. In an example, a device operator can control one or more actuators which push or pull one or both longitudinal members. In an example, one or more actuators which move longitudinal members can be external to a person's body. In an example, one or more actuators which move longitudinal members can be inserted into the person's body (e.g. along with the distal end of the catheter).

In an example, a longitudinal member can be a wire. In an example, a longitudinal member can be a suture, thread, or string. In an example, a longitudinal member can be a chain. In an example, a longitudinal member can be an elastic band. In an example, a longitudinal member can be piezoelectric. In an example, a piezoelectric longitudinal member can be shrunk or elongated by transmission of electrical energy into the member. In an example, differential transmission of electrical energy to first and second piezoelectric longitudinal members can steer an expandable mesh in different directions.

In an example, a first longitudinal member can be attached to a first side of an expandable mesh and a second longitudinal member can be attached to a second side of the expandable mesh. In an example, the second side can be opposite the first side. In an example, four or more longitudinal members can be attached to an expandable mesh, wherein their attachment locations are evenly distributed (e.g. equidistant) around a circumference of the expandable mesh. In an example, a longitudinal member can be attached to a distal portion (e.g. distal circumference) of an expandable mesh. In another example, a longitudinal member can be attached to a central portion (e.g. central circumference) of an expandable mesh.

In this example, longitudinal members are outside (a concavity of) a concave expandable mesh between where they exit a catheter and where they are attached to the expandable mesh. In another example, longitudinal members can be outside (a convexity of) a convex expandable mesh between where they exit a catheter and where they are attached to the expandable mesh. In another example, longitudinal members can be inside (a concavity of) a concave expandable mesh between where they exit a catheter and where they are attached to the expandable mesh. In another example, longitudinal members can be inside (a convexity of) a convex expandable mesh between where they exit a catheter and where they are attached to the expandable mesh.

In an example, a longitudinal member can be detached from an expandable mesh after the mesh has been successfully steered into a proper position within an aneurysm sac. In an example, a longitudinal member can be detached from an expandable mesh after the mesh has been successfully deployed within an aneurysm sac. In an example, a longitudinal member can be detached from an expandable mesh by application of electrical energy to a connection between the longitudinal member and the expandable mesh.

In an example, a longitudinal member can travel through the interior lumen of a catheter to the expandable mesh. In an example, a longitudinal member can travel through a channel in the wall of a catheter to the expandable mesh. In an example, a first longitudinal member can travel through a first channel in the wall of a catheter and a second longitudinal member can travel through a second channel in the wall of the catheter. In an example, a longitudinal member can travel through the space between two coaxial catheters.

In an example, an expandable mesh can self-expand within an aneurysm sac after it exits the catheter. In an example, an expandable mesh (e.g. mesh, lattice, braid, stent, and/or neck bridge) can expand within an aneurysm sac into a bowl, cup, hemispherical, or hemi-elliptical shape. In an example, an expandable mesh can expand within an aneurysm sac into a shape which is section (e.g. the lower half) of a torus. In an example, an expandable mesh can expand within an aneurysm sac into a globular, spherical, or elliptical shape. In an example, an expandable mesh can expand within an aneurysm sac into a funnel or parabolic shape. In an example, an expandable mesh can expand within an aneurysm sac into a hyperbolic or hourglass shape. In an example, an expandable mesh can expand within an aneurysm sac into a radially-asymmetric shape. Relevant variations discussed elsewhere in this disclosure or in priority-linked disclosures can also be applied to this example.

FIG. 16 shows an intrasaccular aneurysm occlusion device comprising: an expandable mesh (e.g. mesh, lattice, braid, stent, and/or neck bridge) 1602 that is inserted and expanded within an aneurysm sac; a catheter 1601 that is inserted into the aneurysm sac, wherein the expandable mesh is inserted through the catheter into the aneurysm sac; and a wire loop 1603 which is attached to the expandable mesh at two locations; wherein the direction, angle, and/or orientation of the expandable mesh can be changed by differentially pulling and/or pushing a first and/or second branch of the wire loop.

FIG. 16 shows this device in a first configuration in which the expandable mesh extends straight out of the catheter (e.g. axially aligned with the longitudinal axis of the catheter). In an example, a device operator can subsequently steer the expandable mesh to the right or left, or up or down, by differentially pulling and/or pushing the first (e.g. left side) and/or second (e.g. right side) loop branches. In this example, a device operator can steer (e.g. tilt or rotate) the expandable mesh to the right by pushing the first (e.g. left side) loop branch and/or pulling the second (e.g. right side) loop branch. In this example, a device operator can steer (e.g. tilt or rotate) the expandable mesh to the left by pulling the first (e.g. left side) loop branch and/or pushing the second (e.g. right side) loop branch.

In an example, a device operator can selectively steer the expandable mesh (e.g. mesh, lattice, braid, stent, and/or neck bridge) in a selected direction, at a selected angle, and/or with a selected orientations by selectively and differentially pulling and/or pushing the first and/or second loop branches. In an example, a device operator can push or pull one or both loop branches manually from a location outside a person's body. In an example, a device operator can control one or more actuators which push or pull one or both loop branches. In an example, one or more actuators which move loop branches can be external to a person's body. In an example, one or more actuators which move loop branches can be inserted into the person's body (e.g. along with the distal end of the catheter).

In an example, a first loop branch can be attached to a first side of an expandable mesh and a second loop branch can be attached to a second side of the expandable mesh. In an example, the second side can be opposite the first side. In an example, four or more loop branches can be attached to an expandable mesh, wherein their attachment locations are evenly distributed (e.g. equidistant) around a circumference of the expandable mesh. In an example, a loop branch can be attached to a distal portion (e.g. distal circumference) of an expandable mesh. In another example, a loop branch can be attached to a central portion (e.g. central circumference) of an expandable mesh.

In this example, loop branches are outside (a concavity of) a concave expandable mesh between where they exit a catheter and where they are attached to the expandable mesh. In another example, loop branches can be outside (a convexity of) a convex expandable mesh between where they exit a catheter and where they are attached to the expandable mesh. In another example, loop branches can be inside (a concavity of) a concave expandable mesh between where they exit a catheter and where they are attached to the expandable mesh. In another example, loop branches can be inside (a convexity of) a convex expandable mesh between where they exit a catheter and where they are attached to the expandable mesh.

In an example, a loop branch can be detached from an expandable mesh after the mesh has been successfully steered into a proper position within an aneurysm sac. In an example, a loop branch can be detached from an expandable mesh after the mesh has been successfully deployed within an aneurysm sac. In an example, a loop branch can be detached from an expandable mesh by application of electrical energy to a connection between the loop branch and the expandable mesh.

In an example, a loop branch can travel through the interior lumen of a catheter to the expandable mesh. In an example, a loop branch can travel through a channel in the wall of a catheter to the expandable mesh. In an example, a first loop branch can travel through a first channel in the wall of a catheter and a second loop branch can travel through a second channel in the wall of the catheter. In an example, a loop branch can travel through the space between two coaxial catheters.

In an example, an expandable mesh can self-expand within an aneurysm sac after it exits the catheter. In an example, an expandable mesh (e.g. mesh, lattice, braid, stent, and/or neck bridge) can expand within an aneurysm sac into a bowl, cup, hemispherical, or hemi-elliptical shape. In an example, an expandable mesh can expand within an aneurysm sac into a shape which is section (e.g. the lower half) of a torus. In an example, an expandable mesh can expand within an aneurysm sac into a globular, spherical, or elliptical shape. In an example, an expandable mesh can expand within an aneurysm sac into a funnel or parabolic shape. In an example, an expandable mesh can expand within an aneurysm sac into a hyperbolic or hourglass shape. In an example, an expandable mesh can expand within an aneurysm sac into a radially-asymmetric shape. Relevant variations discussed elsewhere in this disclosure or in priority-linked disclosures can also be applied to this example.

FIG. 17 shows an intrasaccular aneurysm occlusion device comprising: an expandable mesh (e.g. mesh, lattice, braid, stent, and/or neck bridge) 1703 that is inserted and expanded within an aneurysm sac; a catheter 1701 that is inserted into the aneurysm sac, wherein the expandable mesh is inserted through the catheter into the aneurysm sac; a first flexible longitudinal member (e.g. wire, suture, band, or string) 1702 which is attached to a first portion of the expandable mesh; and a second flexible longitudinal member (e.g. wire, suture, band, or string) 1704 which is attached to a second portion of the expandable mesh; wherein the direction, angle, and/or orientation of the expandable mesh can be changed by differentially pulling and/or pushing the first flexible longitudinal member and/or the second flexible longitudinal member.

FIG. 17 shows this device in a first configuration in which the expandable mesh extends straight out of the catheter (e.g. axially aligned with the longitudinal axis of the catheter). In an example, a device operator can subsequently steer the expandable mesh to the right or left, or up or down, by differentially pulling and/or pushing the first and/or second longitudinal members. In this example, a device operator can steer (e.g. tilt or rotate) the expandable mesh to the right by pushing the first longitudinal member and/or pulling the second longitudinal member. In this example, a device operator can steer (e.g. tilt or rotate) the expandable mesh to the left by pulling the first longitudinal member and/or pushing the second longitudinal member.

In an example, a device operator can selectively steer the expandable mesh (e.g. mesh, lattice, braid, stent, and/or neck bridge) in a selected direction, at a selected angle, and/or with a selected orientations by selectively and differentially pulling and/or pushing the first and/or second longitudinal members. In an example, a device operator can push or pull one or both longitudinal members manually from a location outside a person's body. In an example, a device operator can control one or more actuators which push or pull one or both longitudinal members. In an example, one or more actuators which move longitudinal members can be external to a person's body. In an example, one or more actuators which move longitudinal members can be inserted into the person's body (e.g. along with the distal end of the catheter).

In an example, a longitudinal member can be a wire. In an example, a longitudinal member can be a suture, thread, or string. In an example, a longitudinal member can be a chain. In an example, a longitudinal member can be an elastic band. In an example, a longitudinal member can be piezoelectric. In an example, a piezoelectric longitudinal member can be shrunk or elongated by transmission of electrical energy into the member. In an example, differential transmission of electrical energy to first and second piezoelectric longitudinal members can steer an expandable mesh in different directions.

In an example, a first longitudinal member can be attached to a first side of an expandable mesh and a second longitudinal member can be attached to a second side of the expandable mesh. In an example, the second side can be opposite the first side. In an example, four or more longitudinal members can be attached to an expandable mesh, wherein their attachment locations are evenly distributed (e.g. equidistant) around a circumference of the expandable mesh. In an example, a longitudinal member can be attached to a distal portion (e.g. distal circumference) of an expandable mesh. In another example, a longitudinal member can be attached to a central portion (e.g. central circumference) of an expandable mesh.

In this example, longitudinal members are inside (a concavity of) a concave expandable mesh between where they exit a catheter and where they are attached to the expandable mesh. In another example, longitudinal members can be outside (a concavity of) a concave expandable mesh between where they exit a catheter and where they are attached to the expandable mesh. In another example, longitudinal members can be outside (a convexity of) a convex expandable mesh between where they exit a catheter and where they are attached to the expandable mesh. In another example, longitudinal members can be inside (a convexity of) a convex expandable mesh between where they exit a catheter and where they are attached to the expandable mesh.

In an example, a longitudinal member can be detached from an expandable mesh after the mesh has been successfully steered into a proper position within an aneurysm sac. In an example, a longitudinal member can be detached from an expandable mesh after the mesh has been successfully deployed within an aneurysm sac. In an example, a longitudinal member can be detached from an expandable mesh by application of electrical energy to a connection between the longitudinal member and the expandable mesh.

In an example, a longitudinal member can travel through the interior lumen of a catheter to the expandable mesh. In an example, a longitudinal member can travel through a channel in the wall of a catheter to the expandable mesh. In an example, a first longitudinal member can travel through a first channel in the wall of a catheter and a second longitudinal member can travel through a second channel in the wall of the catheter. In an example, a longitudinal member can travel through the space between two coaxial catheters.

In an example, an expandable mesh can self-expand within an aneurysm sac after it exits the catheter. In an example, an expandable mesh (e.g. mesh, lattice, braid, stent, and/or neck bridge) can expand within an aneurysm sac into a bowl, cup, hemispherical, or hemi-elliptical shape. In an example, an expandable mesh can expand within an aneurysm sac into a shape which is section (e.g. the lower half) of a torus. In an example, an expandable mesh can expand within an aneurysm sac into a globular, spherical, or elliptical shape. In an example, an expandable mesh can expand within an aneurysm sac into a funnel or parabolic shape. In an example, an expandable mesh can expand within an aneurysm sac into a hyperbolic or hourglass shape. In an example, an expandable mesh can expand within an aneurysm sac into a radially-asymmetric shape. Relevant variations discussed elsewhere in this disclosure or in priority-linked disclosures can also be applied to this example.

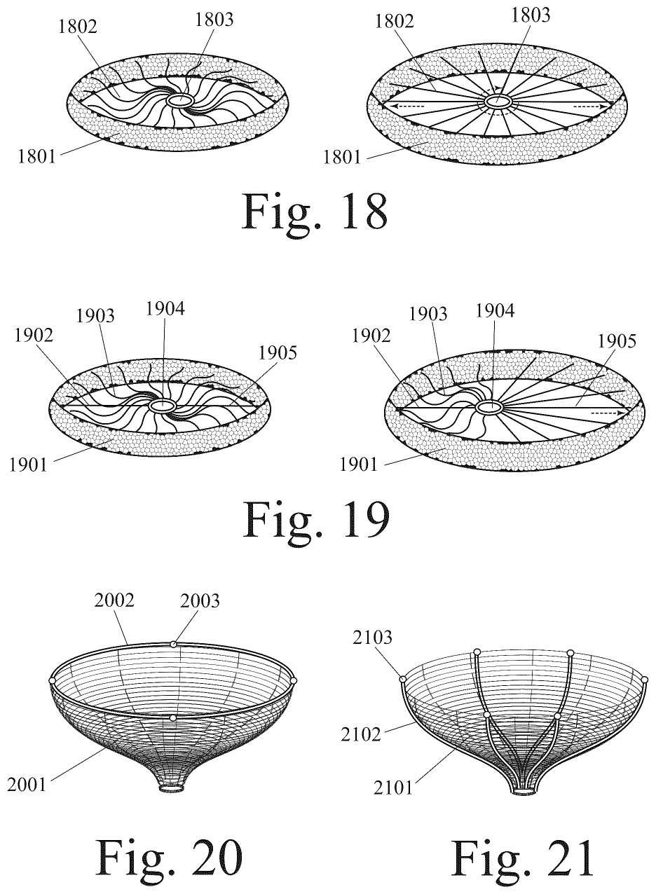

FIG. 18 shows two views, at two different times, of an intrasaccular aneurysm occlusion device comprising: an annular mesh (e.g. mesh, lattice, braid, stent, and/or neck bridge) 1801 that is inserted and expanded within an aneurysm sac; a rotatable hub 1803 at the center of the radial array; and a radial array (e.g. spoke array) of wires 1802 which connect the annular mesh to the hub; wherein the device has a first configuration in which wires in the radial array are more arcuate (e.g. are wavy, undulating, and/or spiraling) and the annular mesh has a first circumference; wherein the device has a second configuration in which wires in the radial array are less arcuate (e.g. are straight) and the annular mesh has a second circumference; wherein the second circumference is greater than the first circumference; and wherein the device is changed from the first configuration to the second configuration by rotation of the hub. The left portion of FIG. 18 shows this device in the first configuration. The right portion of FIG. 18 shows this device in the second configuration. Relevant variations discussed elsewhere in this disclosure or in priority-linked disclosures can also be applied to this example.

FIG. 19 is a ridiculous design, but I am too lazy to renumber the rest of the figures, so here goes. FIG. 19 shows two views, at two different times, of an intrasaccular aneurysm occlusion device comprising: an arcuate mesh (e.g. mesh, lattice, braid, stent, and/or neck bridge) 1901 that is inserted and expanded within an aneurysm sac; a rotatable hub 1904 at the center of the radial array; a radial array (e.g. spoke array) of arcuate (e.g. undulating or spiraling) wires 1903 which connect the arcuate mesh to the hub; and a set of straight wires 1902 and 1905 which connect the arcuate mesh to the hub; wherein the device has a first configuration in which wires in the additional set are all the same length and the annular mesh is radially symmetric (e.g. circular), wherein the device has a second configuration in the lengths of one or more of the wires in the additional set are changed and the annular mesh is radially asymmetric (e.g. not circular). The left portion of FIG. 19 shows this device in the first configuration. The right portion of FIG. 19 shows this device in the second configuration. Relevant variations discussed elsewhere in this disclosure or in priority-linked disclosures can also be applied to this example.

FIG. 20 shows an intrasaccular aneurysm occlusion device comprising: an expandable mesh (e.g. a mesh, lattice, braid, stent, and/or neck bridge) 2001 that is inserted and expanded within an aneurysm sac; a constraint ring 2002 around a portion of the expandable mesh; and a plurality of detachable connections (including 2003) on the constraint ring, wherein selective detachment of a subset of one or more of the detachable connections causes asymmetric radial expansion of the expandable mesh. In an example, the constraint ring does not expand unless sections of it are detached from each other by detachment of one or more detachment connections.

In an example, a detachable connection can be detached by the transmission of electrical energy. In an example, a detachable connection can be melted by the transmission of electrical energy. In an example, detachment of one or more connections on a portion or section of a constraint ring releases a corresponding portion of the expandable mesh from radial constraint by the constraint ring. In an example, a device operator can control asymmetric expansion of the expandable mesh by selecting a specific subset of one or more detachable connections to be detached. In an example, electrical energy can be transmitted to the selected subset of one or more detachable connections to cause selected asymmetric expansion of the expandable mesh.

In an example, portions of the expandable mesh which are released from constraint by the constraint ring due to connection detachments are allowed to expand, while the rest of the expandable mesh remains radially-constrained by the remaining portions of the constraint ring. In an example, a device operator can use selected asymmetric expansion of the expandable mesh to change the shape, orientation, and/or size of the mesh in order to better fit the mesh to the geometry of a specific aneurysm sac. In an example, a device operator can use selected asymmetric expansion of the expandable mesh to change the shape, orientation, and/or size of the mesh in order to better occlude an aneurysm sac with an irregular shape.

In the example, a constraint ring can be on a distal circumference of an expandable mesh. In an example, a constraint ring can be on a central circumference of an expandable mesh. In the example, a constraint ring can be on a distal circumference of a bowl-shaped expandable mesh. In an example, a constraint ring can be on a central circumference of a bowl-shaped expandable mesh. In the example, a constraint ring can be on a distal circumference of a convex (e.g. globular) expandable mesh. In an example, a constraint ring can be on a central circumference of convex (e.g. globular) expandable mesh.

In an example, there can be at least four detachable connections on the circumference of a constraint ring. In an example, detachable connections can be equally-spaced (e.g. equidistant) around the circumference of a constraint ring. In an example, a device can further comprise electroconductive pathways (e.g. microwires) which can transmit electricity to the detachable connections. In an example, there can be one electroconductive pathway (e.g. wire) for each detachable connection, enabling remote selective detachment of a specific subset of individual connections by a device operator.

In an example, selective detachment of one or more detachable connections on the right side of a constraint ring can cause asymmetric expansion of the expandable mesh, wherein the right side of the mesh expands our radially more than the left side of the mesh. In an example, selective detachment of one or more detachable connections on the left side of a constraint ring can cause asymmetric expansion of the expandable mesh, wherein the left side of the mesh expands our radially more than the right side of the mesh. Relevant variations discussed elsewhere in this disclosure or in priority-linked disclosures can also be applied to this example.

FIG. 21 shows an intrasaccular aneurysm occlusion device comprising: an expandable mesh (e.g. a mesh, lattice, braid, stent, and/or neck bridge) 2101 that is inserted and expanded within an aneurysm sac; a plurality of proximal-to-distal constraints (e.g. struts) 2102 along one or more sides of the expandable mesh; and a plurality of detachable connections (including 2103) on the proximal-to-distal constraints, wherein selective detachment of a subset of one or more of the detachable connections causes asymmetric proximal-to-distal expansion of the expandable mesh.

In an example, a detachable connection can be detached by the transmission of electrical energy. In an example, a detachable connection can be melted by the transmission of electrical energy. In an example, detachment of one or more connections on a portion or section of a proximal-to-distal constraint releases a corresponding portion of the expandable mesh from proximal-to-distal constraint by the proximal-to-distal constraint. In an example, a device operator can control asymmetric expansion of the expandable mesh by selecting a specific subset of one or more detachable connections to be detached. In an example, electrical energy can be transmitted to the selected subset of one or more detachable connections to cause selected asymmetric expansion of the expandable mesh.

In an example, portions of the expandable mesh which are released from constraint by the proximal-to-distal constraint due to connection detachments are allowed to expand, while the rest of the expandable mesh remains proximal-to-distally-constrained by the remaining portions of the proximal-to-distal constraint. In an example, a device operator can use selected asymmetric expansion of the expandable mesh to change the shape, orientation, and/or size of the mesh in order to better fit the mesh to the geometry of a specific aneurysm sac. In an example, a device operator can use selected asymmetric expansion of the expandable mesh to change the shape, orientation, and/or size of the mesh in order to better occlude an aneurysm sac with an irregular shape.

In an example, a device can further comprise electroconductive pathways (e.g. microwires) which can transmit electricity to the detachable connections. In an example, there can be one electroconductive pathway (e.g. wire) for each detachable connection, enabling remote selective detachment of a specific subset of individual connections by a device operator. Relevant variations discussed elsewhere in this disclosure or in priority-linked disclosures can also be applied to this example.

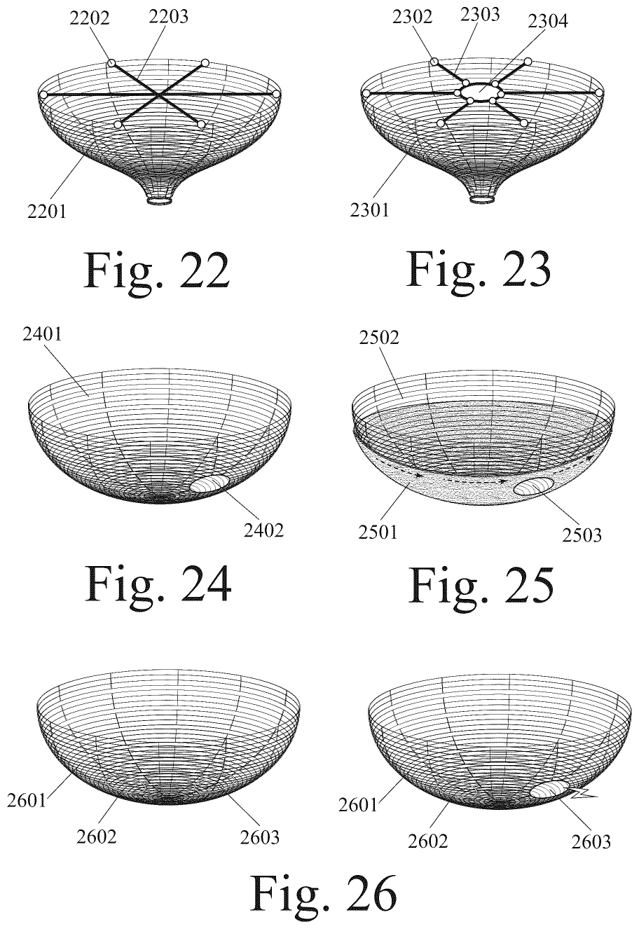

FIG. 22 shows an intrasaccular aneurysm occlusion device comprising: an expandable mesh (e.g. a mesh, lattice, braid, stent, and/or neck bridge) 2201 that is inserted and expanded within an aneurysm sac; a plurality of radial constraints, including 2203, which span a circumference of the expandable mesh; and a plurality of detachable connections, including 2202, which connect the radial constraints to the circumference of the expandable mesh, wherein selective detachment of a subset of one or more of the detachable connections causes asymmetric radial expansion of the expandable mesh.

In an example, a detachable connection can be detached by the transmission of electrical energy. In an example, a detachable connection can be melted by the transmission of electrical energy. In an example, detachment a connection releases a portion of the expandable mesh from radial constraint by a radial constraint. In an example, a device operator can control asymmetric expansion of the expandable mesh by selecting a specific subset of one or more detachable connections to be detached. In an example, electrical energy can be transmitted to the selected subset of one or more detachable connections to cause selected asymmetric expansion of the expandable mesh.

In an example, a portion of an expandable mesh which is released from a radial constraint due to connection detachment is allowed to expand radially, while the rest of the expandable mesh remains radially-constrained by the remaining radial constraints. In an example, a device operator can use selected asymmetric expansion of the expandable mesh to change the shape, orientation, and/or size of the mesh in order to better fit the mesh to the geometry of a specific aneurysm sac. In an example, a device operator can use selected asymmetric expansion of the expandable mesh to change the shape, orientation, and/or size of the mesh in order to better occlude an aneurysm sac with an irregular shape.

In the example, a radial constraint can span a distal circumference of an expandable mesh. In an example, a radial constraint can span a distal opening of an expandable mesh. In an example, a radial constraint can span a distal opening of a concave expandable mesh. In another example, a radial constraint can span a central circumference of an expandable mesh. In an example, there can be at least three radial constraints. In an example, radial constraints can intersect each other at the center of a circumference of the expandable mesh. In an example, each of the radial constraints can span this circumference.

In an example, a device can further comprise electroconductive pathways (e.g. microwires) which can transmit electricity to the detachable connections. In an example, there can be one electroconductive pathway (e.g. wire) for each detachable connection, enabling remote selective detachment of a specific subset of individual connections by a device operator. Relevant variations discussed elsewhere in this disclosure or in priority-linked disclosures can also be applied to this example.

FIG. 23 shows an intrasaccular aneurysm occlusion device comprising: an expandable mesh (e.g. a mesh, lattice, braid, stent, and/or neck bridge) 2301 that is inserted and expanded within an aneurysm sac; a hub 2204 at the center of a circumference of the expandable mesh; a plurality of radial constraints, including 2303, between the circumference of the expandable mesh and the hub; and a plurality of detachable connections, including 2302, which connect the radial constraints to the circumference of the expandable mesh and/or the hub, wherein selective detachment of a subset of one or more of the detachable connections causes asymmetric radial expansion of the expandable mesh.

In an example, a detachable connection can be detached by the transmission of electrical energy. In an example, a detachable connection can be melted by the transmission of electrical energy. In an example, detachment a connection releases a portion of the expandable mesh from radial constraint by a radial constraint. In an example, a device operator can control asymmetric expansion of the expandable mesh by selecting a specific subset of one or more detachable connections to be detached. In an example, electrical energy can be transmitted to the selected subset of one or more detachable connections to cause selected asymmetric expansion of the expandable mesh.

In an example, a portion of an expandable mesh which is released from a radial constraint due to connection detachment is allowed to expand radially, while the rest of the expandable mesh remains radially-constrained by the remaining radial constraints. In an example, a device operator can use selected asymmetric expansion of the expandable mesh to change the shape, orientation, and/or size of the mesh in order to better fit the mesh to the geometry of a specific aneurysm sac. In an example, a device operator can use selected asymmetric expansion of the expandable mesh to change the shape, orientation, and/or size of the mesh in order to better occlude an aneurysm sac with an irregular shape.

In the example, a radial constraint can be connected to a distal circumference of an expandable mesh. In an example, a radial constraint can be connected to a distal opening of a concave expandable mesh. In another example, a radial constraint can be connected to a central circumference of an expandable mesh. In an example, there can be at least six radial constraints. In an example, a device can further comprise electroconductive pathways (e.g. microwires) which can transmit electricity to the detachable connections. In an example, there can be one electroconductive pathway (e.g. wire) for each detachable connection, enabling remote selective detachment of a specific subset of individual connections by a device operator. Relevant variations discussed elsewhere in this disclosure or in priority-linked disclosures can also be applied to this example.

FIG. 24 shows an intrasaccular aneurysm occlusion device comprising an expandable mesh (e.g. a mesh, lattice, braid, stent, and/or neck bridge) 2401 that is inserted and expanded within an aneurysm sac, wherein there is an off-center opening (e.g. opening, hole, lumen, or valve) 2402 in a proximal side (e.g. side or surface) of the expandable mesh through which embolic members and/or material can be inserted.

In an example, an expandable mesh can self-expand within an aneurysm sac after it exits a catheter. In an example, an expandable mesh can be made from braided and/or woven wires. In an example, an expandable mesh can be made from a combination of metal wires and polymer strands. In an example, an expandable mesh can be made by braiding and/or weaving wires and polymer strands together. In another example, an expandable mesh can be made by 3D printing.

In an example, an expandable mesh (e.g. mesh, lattice, braid, stent, and/or neck bridge) can expand into a distal-opening concave shape. In an example, an expandable mesh can expand into a bowl, cup, hemispherical, and/or hemi-ellipsoidal shape. In an example, an expandable mesh can expand within an aneurysm sac into a radially-asymmetric concave shape. In an example, an concave mesh can be formed by compressing, collapsing, and/or inverting a distal portion (e.g. the distal half) of an originally convex mesh into a proximal portion (e.g. the proximal half) of the originally convex mesh.

In an example, an expandable mesh can expand into a convex shape. In an example, an expandable mesh can expand into a globular, spherical, ellipsoidal, or oblate spherical shape. In an example, an expandable mesh can expand into a convex polyhedron shape. In an example, an expandable mesh can expand within an aneurysm sac into a radially-asymmetric convex shape. In an example, an expandable mesh can expand within an aneurysm sac into a funnel or parabolic shape. In an example, an expandable mesh can expand within an aneurysm sac into a hyperbolic or hourglass shape. In an example, an expandable mesh can expand into a toroidal shape. In an example, an expandable mesh can expand into a half-toroidal shape (e.g. like the lower half of a cut bagel).

In an example, a concave expandable mesh can have two layers. In an example, a concave expandable mesh can be a dual-layer mesh. In an example, a concave expandable mesh can have a proximal layer and a distal layer. In an example, a concave mesh can comprise a concave distal layer nested within a proximal concave layer. In an example, an opening (e.g. opening, hole, lumen, or valve) in a concave mesh can be through both distal and proximal layers of a concave mesh. In an example, an opening in a concave mesh can be a composite opening, further comprising a first opening through a distal layer and a second opening through a proximal layer, wherein the first and second openings are aligned.

In an example, a composite expandable mesh can comprise a distal convex (e.g. globular, spherical, ellipsoidal, or oblate spherical) mesh which is at least partially nested within a proximal concave (e.g. bowl, cup, or hemispherical, hemi-ellipsoidal, or funnel shaped) mesh. In an example, a composite expandable mesh (e.g. a mesh, lattice, braid, stent, and/or neck bridge) can comprise: a distally-opening convex (e.g. globular, spherical, ellipsoidal, or oblate spherical) mesh; and a concave (e.g. bowl, cup, or hemispherical, hemi-ellipsoidal, or funnel shaped) mesh; wherein the convex mesh is at least partially nested within the concavity of the concave mesh.

In an example, at least 30% of (the proximal-to-distal axis of) a convex mesh can be nested within the concavity of a concave mesh. In an example, at least 45% of (the proximal-to-distal axis of) a convex mesh can be nested within the concavity of a concave mesh. In an example, an opening through a (composite) expandable mesh can further comprise a first opening (e.g. opening, hole, valve, or lumen) through a convex mesh and a second opening through a concave mesh, wherein the first and second openings are aligned.

In an example, a concave mesh can be nested within a convex mesh. In an example, a concave mesh can be nested within a proximal half of a convex mesh. In an example, an inner convex mesh can be in a proximal half of an interior of an outer convex mesh. In an example, a composite expandable mesh can further comprise a concave (e.g. bowl, cup, or hemispherical, hemi-ellipsoidal, or funnel shaped) mesh within (a proximal half of) a distally-opening convex (e.g. globular, spherical, ellipsoidal, or oblate spherical) mesh.

In an example, a composite expandable mesh (e.g. a mesh, lattice, braid, stent, and/or neck bridge) can comprise: a distally-opening convex (e.g. globular, spherical, ellipsoidal, or oblate spherical) mesh; and a concave (e.g. bowl, cup, or hemispherical, hemi-ellipsoidal, or funnel shaped) mesh; wherein the concave mesh is inside the convex mesh. In an example, an opening in a convex mesh can be through both inner and outer layers of a convex mesh.

In an example, a convex expandable mesh can be a two-layer convex mesh. In an example, a convex expandable mesh can have two layers. In an example, a two-layer convex mesh can further comprise an inner convex mesh and an outer convex mesh. In an example, inner and outer convex meshes can be nested. In an example, inner and outer convex meshes can be concentric. In an example, the centroid of an inner convex mesh can be proximal (e.g. closer to the aneurysm neck) relative to the centroid of an outer convex mesh. In an example, an opening through a (composite) expandable mesh can further comprise a first opening (e.g. opening, hole, valve, or lumen) through an inner convex mesh and a second opening through an outer convex mesh, wherein the first and second openings are aligned.

In an example, a composite expandable mesh can comprise a distal convex (e.g. globular, spherical, ellipsoidal, or oblate spherical) mesh which is at least partially nested within a proximal concave (e.g. bowl, cup, or hemispherical, hemi-ellipsoidal, or funnel shaped) mesh, wherein the proximal concave mesh is less porous, higher mesh density, less elastic, higher durometer, more rigid, and/or more resilient than the distal convex mesh. In an example, a composite expandable mesh can comprise a concave (e.g. bowl, cup, or hemispherical, hemi-ellipsoidal, or funnel shaped) mesh nested inside (the proximal half of) a convex (e.g. globular, spherical, ellipsoidal, or oblate spherical) mesh, wherein the concave mesh is less porous, higher mesh density, less elastic, higher durometer, more rigid, and/or more resilient than the convex mesh.

In an example, an opening (e.g. opening, hole, lumen, or valve) in an expandable mesh can be larger than openings or pores in the rest of the mesh. In an example, an opening (e.g. opening, hole, lumen, or valve) in an expandable mesh can be at least 50% larger than openings or pores in the rest of the mesh. In an example, an opening (e.g. opening, hole, lumen, or valve) in an expandable mesh can be at least 50% larger than openings or pores between braided or woven wires (or strands) in the rest of the mesh.

In an example, a central opening (e.g. opening, hole, lumen, or valve) in an expandable mesh can be larger than openings or pores in the rest of the mesh. In an example, a central opening (e.g. opening, hole, lumen, or valve) in an expandable mesh can be at least 50% larger than openings or pores in the rest of the mesh. In an example, a central opening (e.g. opening, hole, lumen, or valve) in an expandable mesh can be at least 50% larger than openings or pores between braided or woven wires (or strands) in the rest of the mesh.

In an example, a non-central opening (e.g. opening, hole, lumen, or valve) in an expandable mesh can be larger than openings or pores in the rest of the mesh. In an example, a non-central opening (e.g. opening, hole, lumen, or valve) in an expandable mesh can be at least 50% larger than openings or pores in the rest of the mesh. In an example, a non-central opening (e.g. opening, hole, lumen, or valve) in an expandable mesh can be at least 50% larger than openings or pores between braided or woven wires (or strands) in the rest of the mesh.

In an example, the size of a selected opening (e.g. opening, hole, lumen, or valve) in an expandable mesh can be at least 50% larger than the average size of openings or pores in the rest of the mesh. In an example, the size of an opening (e.g. opening, hole, lumen, or valve) in an expandable mesh can be at least 50% larger than the average size of openings or pores between braided or woven wires (or strands) in the rest of the mesh.

In an example, the size of a selected opening (e.g. opening, hole, lumen, or valve) in an expandable mesh can be at least 50% larger than the size of the largest opening or pore elsewhere in the mesh. In an example, the size of an opening (e.g. opening, hole, lumen, or valve) in an expandable mesh can be at least 50% larger than the size of the largest opening or pore between braided or woven wires (or strands) elsewhere in the mesh.

In an example, an opening (e.g. opening, hole, lumen, or valve) in an expandable mesh can allow embolic members and/or material (e.g. coils, beads, string-of-pearls strands, hydrogels, gel, or congealing fluid) to be inserted through the expandable mesh into an aneurysm sac. In another example, an opening (e.g. opening, hole, lumen, or valve) in an expandable mesh allows embolic members and/or material (e.g. coils, beads, string-of-pearls strands, hydrogels, gel, or congealing fluid) to be inserted into an interior space of the expandable mesh.

In another example, an opening (e.g. opening, hole, lumen, or valve) in an expandable convex mesh allows embolic members and/or material (e.g. coils, beads, string-of-pearls strands, hydrogels, gel, or congealing fluid) to be inserted into the interior space of the expandable convex mesh. In another example, an opening (e.g. opening, hole, lumen, or valve) in an expandable two-layer concave mesh allows embolic members and/or material (e.g. coils, beads, string-of-pearls strands, hydrogels, gel, or congealing fluid) to be inserted between the layers of the concave mesh.

In another example, an opening (e.g. opening, hole, lumen, or valve) through a two-layer expandable mesh can comprise a first opening through a first layer and a second opening through a second layer, wherein the two openings are aligned. In another example, an opening through a two-part expandable mesh can comprise a first opening through an inner mesh and a second opening through an outer mesh, wherein the two openings are aligned. In another example, an opening through a two-part expandable mesh can comprise a first opening through an proximal mesh and a second opening through a distal mesh, wherein the two openings are aligned. In another example, an opening through a two-part expandable mesh can comprise a first opening through an proximal concave mesh and a second opening through a distal convex mesh, wherein the two openings are aligned.

In another example, an opening (e.g. opening, hole, lumen, or valve) in an expandable mesh can in a non-central location. In an example, a non-central opening in an expandable mesh is at a location other than the central (radial) axis of the mesh. In another example, a non-central opening (e.g. opening, hole, lumen, or valve) in an expandable mesh can be through a proximal side and/or surface of the mesh. In an example, a non-central opening in an expandable mesh can be through the proximal half of the mesh. In an example, a non-central opening in an expandable mesh can be through the proximal third of the mesh. In an alternative example, an opening in an expandable mesh can be in a central location (e.g. at the central radial axis of the mesh).

In an example, an opening in an expandable mesh can comprise a fixed-diameter hole in the mesh. In an example, the size (e.g. diameter) of an opening in an expandable mesh can be variable. In an example, the size (e.g. diameter) of an opening in an expandable mesh can automatically decrease after embolic members and/or material has been inserted through it. In an example, an opening in an expandable mesh can comprise a (one-way) value which automatically closes after embolic members and/or material has been inserted through it. In another example, a device operator can control the operation of a valve remotely.

In an example, the size (e.g. diameter) of an opening in an expandable mesh can be remotely changed by a device operator. In an example, a device operator can change (e.g. adjust) the size of an opening in an expandable mesh by pulling, pushing, or rotating a wire connected to the opening. In an example, a device can further comprise an (electromagnetic) actuator which changes (e.g. adjusts) the size of an opening in an expandable mesh, wherein a device operating can change (e.g. adjust) the size of the opening by remotely controlling the actuator. In an example, an opening in an expandable mesh can be closed by the transmission of electrical energy (e.g. to one or more radial constraints on the opening), wherein a device operating can remotely close the opening by controlling this transmission of electrical energy.

In an example, there can be a plurality of openings (e.g. opening, hole, lumen, or valve) on the proximal side /r surface) of an expandable mesh through which embolic members and/or material can be inserted. In an example, there can be a plurality of non-central openings (e.g. opening, hole, lumen, or valve) on the proximal side (or surface) of an expandable mesh through which embolic members and/or material can be inserted. In an example, there can be three or more non-central openings on an expandable mesh through which embolic members and/or material can be inserted. In an example, there can be a plurality of openings on an expandable mesh through which embolic members and/or material can be inserted. In an example, there can be one central opening in the proximal side (or surface) of an expandable mesh and three or more non-central openings in the expandable mesh around the central opening.

In an example, an expandable mesh can have a plurality of adjustable openings, wherein a subset of one or more of these openings can be selectively opened by a device operator to insert embolic members and/or material through the expandable mesh. In an example, a subset of openings can be remotely opened by a device operator via targeted transmission of electrical energy. In an example, a subset of openings can be remotely opened by a device operator via targeted transmission of light energy. In an example, a subset of openings can be remotely opened by a device operator via targeted transmission of thermal energy. In an example, a subset of openings can be remotely opened by a device operator via pulling, pushing, or rotating a wire. In an example, a subset of openings can be remotely opened by a device operator via selective activation of one or more (electromagnetic) actuators which control the openings. Relevant variations discussed elsewhere in this disclosure or in priority-linked disclosures can also be applied to this example.

FIG. 25 shows an intrasaccular aneurysm occlusion device comprising: an expandable mesh (e.g. a mesh, lattice, braid, stent, and/or neck bridge) 2502 that is inserted and expanded within an aneurysm sac; and a rotatable bowl or disk 2501 on the proximal side of the expandable mesh, wherein there is at least one off-center opening (e.g. opening, hole, lumen, or valve) 2503 in the rotatable bowl or disk through which embolic members and/or material can be inserted. In an example, a device operator can change the location of the opening by rotating the bowl or disk. Variations discussed with respect to the device shown in FIG. 24 can also be applied to this device. Relevant variations discussed elsewhere in this disclosure or in priority-linked disclosures can also be applied to this example.

FIG. 26 shows two views, at two different times, of an intrasaccular aneurysm occlusion device comprising an expandable mesh (e.g. a mesh, lattice, braid, stent, and/or neck bridge) 2601 that is inserted and expanded within an aneurysm sac, wherein there is a plurality of off-center openings (e.g. openings, holes, lumens, or valves) 2602 and 2603 in a proximal side (e.g. side or surface) of the expandable mesh through which embolic members and/or material can be inserted, wherein a subset of one or more of the off-center openings can be selectively opened or closed by a device operator.

The left portion of FIG. 26 shows this device at a first time when all of the off-center openings are closed. The right portion of FIG. 26 shows this device at a second time when a selected subset of one or more of the off-center openings have been opened. In an example, a device operator can selectively open or close one or more of the plurality of off-center openings by selective transmission of electrical energy to one or more of them. Variations discussed with respect to the device shown in FIG. 24 can also be applied to this device. Relevant variations discussed elsewhere in this disclosure or in priority-linked disclosures can also be applied to this example.

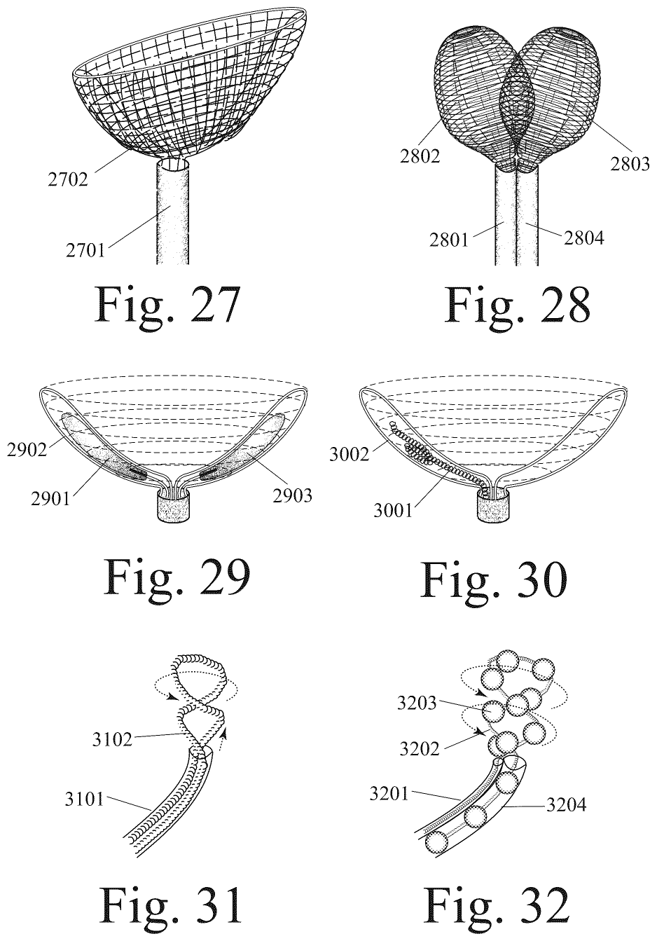

FIG. 27 shows an intrasaccular aneurysm occlusion device comprising: a radially-asymmetric expandable mesh (e.g. a mesh, lattice, braid, stent, and/or neck bridge) 2702 that is inserted and expanded within an aneurysm sac; a catheter 2701 that is inserted into the aneurysm sac, wherein the expandable mesh is inserted through the catheter into the aneurysm sac.

In an example, a radially-asymmetric expandable mesh can be a concave mesh with a central axis which intersects a longitudinal axis of the catheter at an acute angle. In an example, a radially-asymmetric expandable mesh can be a convex mesh with a central axis which intersects a longitudinal axis of the catheter at an acute angle. In an example, there can be a first distance between a catheter and the right side of a radially-asymmetric mesh and a second distance between the catheter and the left side of the radially-asymmetric mesh, wherein the first distance is greater than the second distance. Relevant variations discussed elsewhere in this disclosure or in priority-linked disclosures can also be applied to this example.

FIG. 28 shows an intrasaccular aneurysm occlusion device comprising: a dual catheter that is inserted into an aneurysm sac, wherein the dual catheter further comprises a first catheter 2801 and a second catheter 2804, and wherein the first catheter and the second catheter are parallel to each other; a first expandable mesh (e.g. a mesh, lattice, braid, stent, and/or neck bridge) 2802 which is inserted into the aneurysm sac through the first catheter; and a second expandable mesh (e.g. a mesh, lattice, braid, stent, and/or neck bridge) 2803 which is inserted into the aneurysm sac through the second catheter.

In an example, a device operator can steer the first and second expandable meshes in a selected direction by differentially inserting the first and second expandable meshes. In an example, a device operator can steer the first and second expandable meshes in a selected direction by inserting the first and second expandable meshes by different amounts. In an example, a device operator can direct the first and second expandable meshes in a selected direction, at a selected angle, or with a selected orientation by inserting the first and second expandable meshes to different extents and/or at different times. In an example, one or both of the expandable meshes can be convex. Relevant variations discussed elsewhere in this disclosure or in priority-linked disclosures can also be applied to this example.

FIG. 29 shows an intrasaccular aneurysm occlusion device comprising: a multi-layer concave expandable mesh (e.g. a mesh, lattice, braid, stent, and/or neck bridge) 2902 which is inserted and expanded within an aneurysm sac; and a plurality of fluid-filled chambers 2901 and 2903 between layers of the multi-layer concave expandable mesh, wherein a first chamber in the plurality of fluid-filled chambers is filled more than a second chamber in the plurality of fluid-filled chambers, thereby causing the multi-layer concave expandable mesh to become radially-asymmetric.

In an example, a device operator can cause a multi-layer concave mesh to become radially-asymmetric by filling one chamber between the layers of the mesh more than another chamber between the layers of the mesh. In an example, a device operator can steer the concave mesh in a selected direction, at a selected angle, and/or with a selected orientation by selectively filling one or more fluid-filled chambers more than other fluid-filled chambers. In an example, the device can further comprise a fluid-transmitting lumen for each of the fluid-filled chambers, thereby enabling selective filling of one or more chambers in the plurality of fluid-filled chambers.

In an example, a multi-layer concave expandable mesh can have two layers. In an example, a multi-layer concave mesh can have a proximal layer and a distal layer. In an example, a plurality of fluid-filled chambers can be located between the proximal layer and the distal layer. In an example, a first fluid-filled chamber can be located on a first side (e.g. to the left of a central axis) of a multi-layer concave mesh and a second fluid-filled chamber can be located on a second side (e.g. to the right of the central axis) of the multi-layer concave mesh.

In an example, a fluid-filled chamber can be a balloon. In an example, a fluid-filled chamber can be filled with a liquid or gel. In an example, a fluid-filled chamber can be filled with a gas. In an example, a fluid-filled chamber can span between 25% and 75% of the proximal-to-distal axial distance of a multi-layer concave expandable mesh. In an example, there can be three or more fluid-filled chambers between the layers of a multi-layer concave expandable mesh. In an example, a plurality of fluid-filled chambers can be equally-distributed around the circumference of a multi-layer concave expandable mesh.

In an example, a multi-layer concave expandable mesh (e.g. mesh, lattice, braid, stent, and/or neck bridge) can expand within an aneurysm sac into a bowl, cup, hemispherical, or hemi-elliptical shape. In an example, a multi-layer concave mesh can be formed by compressing, collapsing, and/or inverting a distal portion (e.g. the distal half) of an originally-convex mesh into a proximal portion (e.g. the proximal half) of the originally-convex mesh. Relevant variations discussed elsewhere in this disclosure or in priority-linked disclosures can also be applied to this example.

FIG. 30 shows an intrasaccular aneurysm occlusion device comprising: a multi-layer concave expandable mesh (e.g. a mesh, lattice, braid, stent, and/or neck bridge) 3002 which is inserted and expanded within an aneurysm sac; and embolic members and/or material 3001 which is inserted between layers of the multi-layer concave expandable mesh, wherein insertion embolic members and/or material in a radially-asymmetric manner (e.g. more on one side of the mesh than the other) causes the multi-layer concave expandable mesh to become radially-asymmetric.

In an example, a device operator can cause a multi-layer concave mesh to become radially-asymmetric by filling a space between layers on a first side of the mesh with more embolic members and/or material than a space between layers on a second side of the mesh. In an example, a device operator can steer a multi-layer concave mesh in a selected direction, at a selected angle, and/or with a selected orientation by selectively and differentially filling different radial portions of the space between layers of the mesh.

In an example, a multi-layer concave expandable mesh can have two layers. In an example, a multi-layer concave mesh can have a proximal layer and a distal layer. In an example, embolic members and/or material can be inserted between the proximal layer and the distal layer. In an example, embolic members and/or material can be selected from the group consisting of: embolic coils, embolic beads or spheres, string-of-pearls embolic strands, microsponges, hydrogels, gel, and congealing liquid. In an example, there can be one or more openings or holes in the proximal side (or surface) of an concave mesh through which embolic members and/or material can be inserted into different locations between layers of a multi-layer concave mesh.

In an example, a multi-layer concave expandable mesh (e.g. mesh, lattice, braid, stent, and/or neck bridge) can expand within an aneurysm sac into a bowl, cup, hemispherical, or hemi-elliptical shape. In an example, a multi-layer concave mesh can be formed by compressing, collapsing, and/or inverting a distal portion (e.g. the distal half) of an originally-convex mesh into a proximal portion (e.g. the proximal half) of the originally-convex mesh. Relevant variations discussed elsewhere in this disclosure or in priority-linked disclosures can also be applied to this example.

FIG. 31 shows an intrasaccular aneurysm occlusion device comprising: a catheter 3101 that is inserted into an aneurysm sac; and a coil loop 3102 that this is inserted through the catheter into the aneurysm sac, wherein the coil loop further comprises a first strand (e.g. strand, branch, or section) and a second strand (strand, branch, or section), and wherein rotation of the first strand and/or the second strand causes the first strand and the second strand to wind around each other and form an entwined mass within the aneurysm sac.

In an example, rotation of the first and/or second strands (e.g. strands, branches, or sections) can be done while the coil loop is being inserted into the aneurysm sac. In an example, rotation of the first and/or second strands can be done after the coil loop has been inserted into the aneurysm. In an example, rotation of first and/or second strands can steer a coil loop in a desired direction. In an example, a device operator can manually rotate one or both of the strands from outside a person's body. In an example, one or both of the strands can be automatically rotated by an (electromagnetic) actuator.

In an example, a loop which is inserted into an aneurysm sac can comprise one or more string-of-pearls strands, wherein a string-of-pearls strand further comprises a longitudinal series of embolic components (e.g. beads, microspheres, microsponges, or hydrogels) which are connected to each other by one or more wires, sutures, filaments, strings, or coils. In an example, embolic components in a string-of-pearls strand can attach (e.g. stick, adhere, or interdigitate) to each other when the strands of the loop are intertwined. Relevant variations discussed elsewhere in this disclosure or in priority-linked disclosures can also be applied to this example.

FIG. 32 shows an intrasaccular aneurysm occlusion device comprising: a dual catheter that is inserted into an aneurysm sac, wherein the dual catheter further comprises a first catheter 3201 and a second catheter 3204, and wherein the first catheter and the second catheter are parallel to each other; and a string-of-pearls loop that is inserted through the catheter into the aneurysm sac, wherein the string-of-pearls loop further comprises a first strand (e.g. strand, branch, or section) and a second strand (e.g. strand, branch, or section), and wherein rotation of the first strand and/or the second strand causes the first strand and the second strand to wind around each other and form an entwined mass within the aneurysm sac. In an example, a string-of-pearls can comprise a longitudinal series of embolic components (e.g. beads, microspheres, microsponges, or hydrogels) 3203 which are connected to each other by one or more wires, sutures, filaments, strings, springs, or coils 3202.

In an example, a loop which is inserted into an aneurysm sac can comprise string-of-pearls strands, wherein a string-of-pearls strand further comprises a longitudinal series of embolic components which are (pairwise) connected to each other by one or more longitudinal wires, sutures, filaments, strings, springs, or coils. In an example, the embolic components can be beads or microspheres. In an example, the embolic components can be microsponges or hydrogels. In an example, the embolic components can have globular shapes. In an example, the embolic components can have polyhedron shapes.

In an example, longitudinal members which interconnect embolic components in longitudinal series in a string-of-pearls strand can be wires. In an example, longitudinal members which interconnect embolic components in longitudinal series in a string-of-pearls strand can be sutures, filaments, threads, or strings. In an example, longitudinal members which interconnect embolic components in longitudinal series in a string-of-pearls strand can be coils or chains.

In an example, each pair of proximal embolic components in a longitudinal series can be interconnected by a single longitudinal member (e.g. single wire, suture, filament, string, cord, spring, or coil). In an example, embolic components in a longitudinal series can be centrally connected by one or more longitudinal members. In an example, the longitudinal members can pass through the centers (e.g. centroids) of the embolic components. In an example, each pair of proximal embolic components in a longitudinal series can be interconnected by two or more longitudinal member (e.g. single wire, suture, filament, string, cord, spring, or coil). In an example, a longitudinal member (e.g. wire) which interconnects embolic components in a string-of-pearls can be straight as it passes through a catheter to an aneurysm sac. In an example, a longitudinal member (e.g. wire) which interconnects embolic components in a string-of-pearls can be undulating (e.g. sinusoidal) as it passes through a catheter to an aneurysm sac.

In an example, embolic components in a string-of-pearls series can be equidistant. In an example, embolic components in a string-of-pearls series can all have the same durometer level and/or Shore value. In an example, embolic components in string-of-pearls strands can all be the same size. In an example, distal embolic components in a string-of-pearls series can be larger than proximal embolic components in the string-of-pearls series. In an example, distal embolic components in a string-of-pearls series can be closer together than proximal embolic components in the string-of-pearls series.

In an example, distal embolic components in a string-of-pearls series can have higher durometer levels and/or Shore values than proximal embolic components in the string-of-pearls series. In an example, distal embolic components in a string-of-pearls series can be smaller than proximal embolic components in the string-of-pearls series. In an example, distal embolic components in a string-of-pearls series can be farther apart than proximal embolic components in the string-of-pearls series. In an example, distal embolic components in a string-of-pearls series can have lower durometer levels and/or Shore values than proximal embolic components in the string-of-pearls series.

In an example, the embolic components in a string-of-pearls strand can attach (e.g. stick, adhere, or interdigitate) to each other when the string-of-pearls strands of the loop are intertwined. In an example, embolic components in a string-of-pearls loop can attach (e.g. stick, adhere, or interdigitate) to each other when strands of the loop are intertwined. In an example, embolic components in a string-of-pearls series can have protrusions which interdigitate with (e.g. attach to) each other in order to attached embolic components to each other as they are rotated in a loop.

In this example, embolic components in a first string-of-pearls strand (e.g. strand, branch, or section of a loop) can be larger than embolic components in a second string-of-pearls strand. In this example, embolic components in a first string-of-pearls strand (e.g. strand, branch, or section of a loop) can be closer together than embolic components in a second string-of-pearls strand. In an example, the first catheter can be wider can be wider than the second catheter.

In an example, rotation of the first and or second strands (e.g. strands, branches, or sections) can be done while the string-of-pearls loop is being inserted into the aneurysm sac. In an example, rotation of the first and or second strands can be done after the string-of-pearls loop has been inserted into the aneurysm. In an example, rotation of the first and or second string-of-pearls strands (e.g. string-of-pearls strands, branches, or sections) can be done while the string-of-pearls loop is being inserted into the aneurysm sac. In an example, rotation of the first and or second string-of-pearls strands can be done after the string-of-pearls loop has been inserted into the aneurysm.

In an example, a device operator can manually rotate one or both of the strands from outside a person's body. In an example, one or both of the strands can be automatically rotated by an (electromagnetic) actuator. In an example, one or both of the strands can be automatically rotated by an (electromagnetic) actuator which is controlled by the device operator. In an example, by pushing first and second strands of a string-of-pearls loop by different amounts, a device operator can steer the loop in a selected direction, at a selected angle, and or with a selected orientation. In an example, differential insertion and or extension of first and second strands of a string-of-pearls loop can steer the loop in a selected direction, at a selected angle, and or with a selected orientation. In an example, rotation of first and or second strands can steer a string-of-pearls loop in a desired direction. Relevant variations discussed elsewhere in this disclosure or in priority-linked disclosures can also be applied to this example.

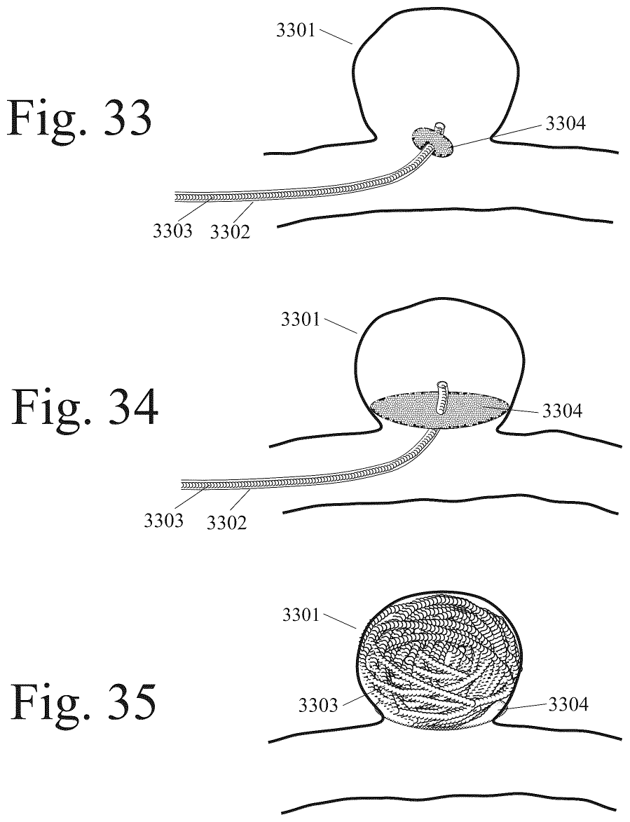

FIGS. 33 through 35 show three views, at three different times, of an intrasaccular aneurysm occlusion device comprising: a toroidal expandable mesh (e.g. mesh, lattice, braid, stent, and/or neck bridge) 3304 which is inserted and expanded within an aneurysm sac 3301, wherein the mesh expands into a torus shape; a catheter 3302 which is inserted into the aneurysm sac, wherein the expandable mesh is delivered through the catheter to the aneurysm sac; and one or more embolic coils 3303 which are inserted into the aneurysm sac through a central opening in the torus shape of the expandable mesh. In an example, this device (or method) can be described as aneurysm coil jailing using an expandable toroidal mesh.

FIG. 33 shows this device at a first time, before the expandable mesh has been expanded within the aneurysm sac. FIG. 34 shows this device at a second time, after the expandable mesh has been expanded within the aneurysm sac, but before the one or more embolic coils have been inserted through the center of the toroidal expandable mesh into the aneurysm sac. FIG. 35 shows this device at a third time, after the expandable mesh has been expanded within the aneurysm sac and after the one or more embolic coils have been inserted through the center of the toroidal expandable mesh into the aneurysm sac.

FIGS. 33 through 35 can also be described as an intrasaccular aneurysm occlusion device comprising: a toroidal member (e.g. toroidal mesh, lattice, braid, stent, and/or neck bridge) that is inserted into an aneurysm sac to substantially occlude the neck of the aneurysm; a longitudinal flexible embolic member (e.g. an embolic coil) that is inserted into the aneurysm sac through a central opening of the toroidal member, wherein accumulation of the longitudinal flexible embolic member occludes the aneurysm sac; and an embolic-member-delivering lumen (e.g. catheter), wherein the longitudinal flexible embolic member travels through the lumen in order to be inserted into the aneurysm sac.

FIGS. 33 through 35 can also be described as an intrasaccular aneurysm occlusion device comprising: a catheter that is inserted into a blood vessel which is the parent vessel from which an aneurysm sac has formed; a longitudinal flexible embolic member that is configured to travel through the catheter and be inserted into the aneurysm sac; and an expandable toroidal member that is configured to travel through the catheter, be inserted into the aneurysm sac, and be expanded within the aneurysm sac; wherein this toroidal member substantially occludes the aneurysm neck after it is expanded; wherein the longitudinal flexible embolic member is inserted into the aneurysm sac through the central opening of the expandable toroidal member; and wherein the expandable toroidal member prevents the longitudinal flexible member from protruding into the parent vessel of the aneurysm.

In an example, a toroidal mesh can self-expand within an aneurysm sac after exiting a catheter. In an example, a toroidal member (e.g. expandable mesh) can be expanded within an aneurysm sac by being filled with a flowable substance (e.g. a liquid, gas, or gel) after exiting a catheter. In an example, a toroidal member (e.g. expandable mesh) can be remotely expanded within an aneurysm sac by an device operator by being filled with a flowable substance (e.g. a liquid, gas, or gel) after exiting a catheter. In an example, a toroidal member (e.g. expandable balloon) can be inflated within an aneurysm sac after exiting a catheter. In an example, a toroidal mesh can be expanded by inflation of a balloon within the mesh.

In an example, the catheter can be inserted through the central opening of the toroidal expandable mesh in order to insert the one or more embolic coils into the aneurysm sac. In an example, the diameter of the central opening of the torus shape of the expandable mesh can be between 5% and 15% of the diameter of the expandable mesh. In an example, the diameter of the central opening of the torus shape of the expandable mesh can be between 10% and 25% of the diameter of the expandable mesh.

In an example, the device can further comprise a valve in the central opening of the toroidal expandable mesh. In an example, a device operator can remotely open or close this valve. In an example, this valve can be a one-way value which allows one or more coils to be inserted into the aneurysm sac but does not allow them to escape out of the sac. In another example, embolic members and/or material other than coils can be inserted through the central opening of the toroidal expandable mesh in order to occlude the aneurysm sac. In an example, embolic beads, microsponges, or hydrogels can be inserted through the central opening of the toroidal expandable mesh (instead of coils) in order to occlude the aneurysm sac. Relevant variations discussed elsewhere in this disclosure or in priority-linked disclosures can also be applied to this example.

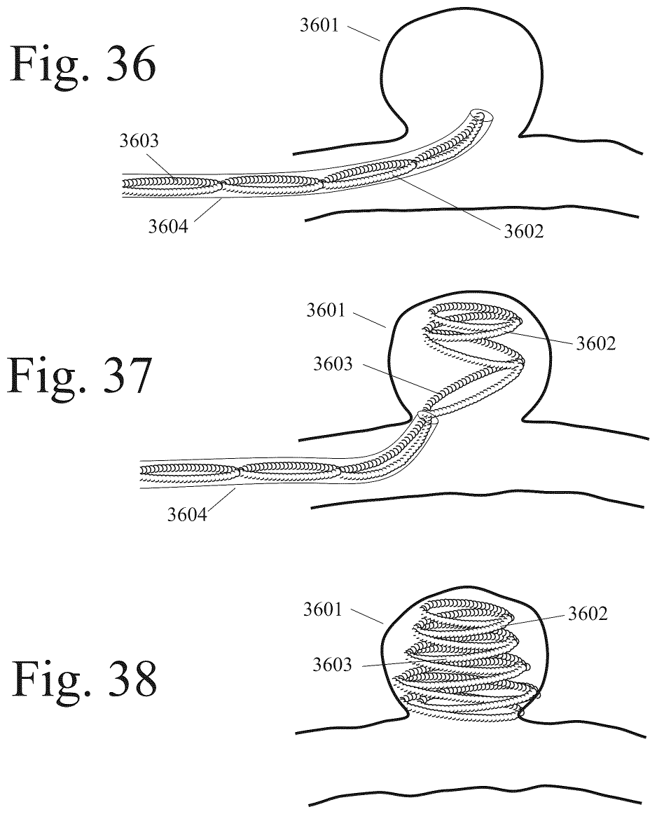

FIGS. 36 through 38 show three views, at three different times, of an intrasaccular aneurysm occlusion device comprising: a longitudinal series of embolic coil loops (including loops 3602 and 3603) that are inserted into an aneurysm sac 3601; and a catheter 3604 through which the coil loops are delivered to the aneurysm sac. FIG. 36 shows this device at a first time, before any embolic coil loops have been inserted into the aneurysm sac. FIG. 37 shows this device at a second time, after some embolic coil loops have been inserted into the aneurysm sac. FIG. 38 shows this device at a third time, after embolic coil loops have been inserted into the aneurysm sac and the catheter has been removed.

In an example, a longitudinal series of coil loops can be made by pair-wise connecting a plurality of separately-formed coil loops. In an example, a longitudinal series of coil loops can be made by connecting two separate coils together at a series of longitudinal locations. In another example, instead of being separate coil loops which are connected to each other, a longitudinal series of coil loops can be formed from two continuous coils which are connected together at different longitudinal locations to form a longitudinal series of coils loops. In an example, these connection locations can be equally spaced along the lengths of the two coils. In an example, the device can further comprise fabric and or mesh spanning the interiors of the coil loops.

In an example, a diameter of a coil loop in the longitudinal series of coil loops can be equal to the central and or maximum diameter of the aneurysm sac. In an example, the diameter of a coil loop in the longitudinal series of coil loops can be between 80% and 100% of the central and or maximum diameter of the aneurysm sac. In an example, the diameter of a coil loop in the longitudinal series of coil loops can be greater than the diameter of the aneurysm neck. In an example, the diameter of the largest coil loop in the longitudinal series of coil loops can be equal to the central and or maximum diameter of the aneurysm sac.

In an example, a diameter of the largest coil loop in the longitudinal series of coil loops can be between 80% and 100% of the central and or maximum diameter of the aneurysm sac. In an example, the diameter of the largest coil loop in the longitudinal series of coil loops can be greater than the diameter of the aneurysm neck. In an example, the diameter of the smallest coil loop in the longitudinal series of coil loops can be greater than the diameter of the aneurysm neck. In an example, distal coil loops in a longitudinal series of coil loops can be larger than proximal coil loops in the series. In an example, distal coil loops in a longitudinal series of coil loops can be smaller than proximal coil loops in the series.

In an example, in each pair of adjacent loops in the longitudinal series, the adjacent loops are connected to each other at a single location. In an example, adjacent loops in the longitudinal series can be colinear during delivery through the catheter, but their best-fitting virtual planes can intersect at acute angles after they exit the catheter. In an example, adjacent loops in the longitudinal series can be colinear during delivery through the catheter, but their best-fitting virtual planes can intersect at angles between 10 and 45 degrees after they exit the catheter. In an example, an embolic loop is collapsed during delivery through the catheter so that the two branches of the loop are substantially parallel to each other during delivery through the catheter. However, the embolic loop expands after exiting the catheter to form a convex (e.g. elliptical, oval, oblate circular, or oblate circular) shape.

In an example, accumulating loops in an aneurysm sac can form an embolic mass with a zigzag pattern, wherein acute angles formed between the zigzagging loops are less than 30 degrees. In an example, the plane of a coil loop can be within 15 degrees of being parallel to the plane of the central circumference of the aneurysm neck. In an example, the plane of a coil loop can be within 15 degrees of being parallel to the plane of the central circumference of the aneurysm sac. In an example, the plane of a coil loop can be within 30 degrees of being parallel to the plane of the central circumference of the aneurysm neck.

In an example, the plane of a coil loop can be within 30 degrees of being parallel to the plane of the central circumference of the aneurysm sac. In an example, accumulating loops in an aneurysm sac can form a beehive-shaped mass. In an example, accumulating loops in an aneurysm sac can form an embolic mass with a zigzag pattern. In an example, the orientations of sequential loops in the longitudinal series of coil loops can alternate sequentially (e.g. from side to side) when deployed in the aneurysm sac. Relevant variations discussed elsewhere in this disclosure or in priority-linked disclosures can also be applied to this example.

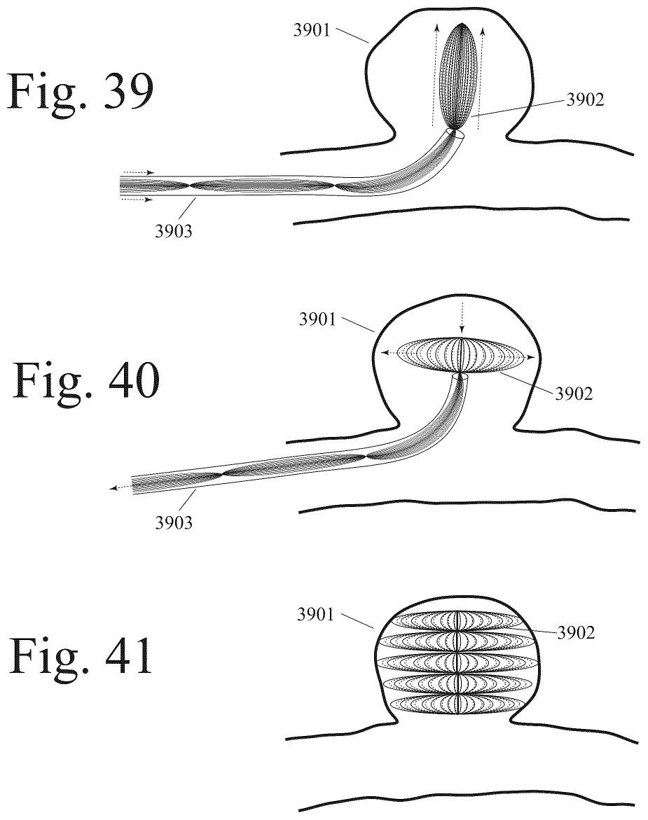

FIGS. 39 through 41 show three views, at three different times, of an intrasaccular aneurysm occlusion device comprising: a series of connected expandable meshes (e.g. meshes, lattices, braids, and/or stents) 3902 which are inserted and expanded within an aneurysm sac 3901; and a catheter 3903 through which the expandable meshes are delivered to the aneurysm sac, wherein expandable meshes in the series have a radially-compressed and longitudinally-lengthened first configuration when they are in the catheter being delivered to the aneurysm sac, and wherein expandable meshes in the series have a radially-expanded and longitudinally-shortened second configuration after they exit the catheter in the aneurysm sac.

FIG. 39 shows this device at a first time, when a first expandable mesh in the series has been inserted into the aneurysm sac, but not yet radially-expanded. FIG. 40 shows this device at a second time, after the first expandable mesh has been radially-expanded in the aneurysm sac. FIG. 41 shows this device at a third time, after all of the expandable meshes in the series have been inserted and radially-expanded within the aneurysm sac.

In an example, expandable meshes in a series can self-expand within the aneurysm sac. In an example, one or more of the expandable meshes in a series can be convex. In an example, one or more of the expandable meshes in a series can have globular, ellipsoidal, ovaloid, oblate spherical, or spherical shapes after expansion with the aneurysm sac. In an example, one or more of the expandable meshes in a series can be concave. In an example, one or more of the expandable meshes in a series can have bowl, cup, hemispherical, or semi-ellipsoidal shapes after expansion with the aneurysm sac. In an example, expandable meshes can have disk shapes. In an example, expandable meshes can have toroidal shapes.

In an example, one or more distal expandable meshes in a series can have convex shapes and one or more proximal expandable meshes in the series can have concave shapes. In an example, one or more distal expandable meshes in a series can have concave shapes and one or more proximal expandable meshes in the series can have convex shapes. In an example, two or more of the expandable meshes in a series can be nested. In an example, distal expandable meshes in a series can be smaller than proximal expandable meshes in the series. In an example, proximal expandable meshes in a series can be smaller than proximal expandable meshes in the series.

In an example, a series of expandable meshes can be formed by directly connecting a plurality of separately-formed expandable meshes together. In an example, a series of expandable meshes can be formed by directly connecting a plurality of separately-formed expandable meshes together, wherein (virtual extensions of) the central axes of these meshes are colinear. In an example, a plurality of separately-formed expandable meshes can be connected together by a mechanism selected from the group consisting of: adhesion, soldering, welding, crimping, pinching, tying, braiding, weaving, snapping, clipping, and hooking. In an example, expandable meshes can be connected to each other along their central axes (e.g. the axes which are their central longitudinal axes as they travel through the catheter).

In an example, a series of expandable meshes can be formed by indirectly connecting a plurality of separately-formed expandable meshes with a wire, string, cord, cord, suture, coil, or filament. In an example, expandable meshes can be connected together along their central axes by a wire, string, cord, cord, suture, coil, or filament. In an example, expandable meshes can be connected together by passing a wire, string, cord, cord, suture, coil, or filament through their central axes.

In an example, distal expandable meshes in a series can be smaller (e.g. have smaller diameters) than proximal expandable meshes in the series. In an example, distal expandable meshes in a series can have lower durometer levels and/or Shore values than proximal expandable meshes in the series. In an example, distal expandable meshes in a series can be more elastic and/or flexible than proximal expandable meshes in the series.

In an example, distal expandable meshes in a series can be more porous and/or have lower mesh densities than proximal expandable meshes in the series. In an example, distal expandable meshes in a series can be closer together than proximal expandable meshes in the series. In an example, distal expandable meshes in a series can be larger (e.g. have larger diameters) than proximal expandable meshes in the series. In an example, distal expandable meshes in a series can be farther apart than proximal expandable meshes in the series.

In an example, a series of expandable meshes can be formed by radially constraining a continuous tubular mesh at different locations along the longitudinal axis of the tubular mesh. In an example, a series of expandable meshes can comprise a longitudinal mesh with a plurality of radial undulations which expand when released from a catheter. In an example, a tubular mesh can be radially constrained at different locations by annular bands, rings, washers, or coils. In an example, a tubular mesh can be radially constrained at different locations by circular wires, twist ties, strings, or sutures. In an example, a tubular mesh can be radially constrained at different locations by welding, melting, and/or soldering. In an example, a tubular mesh can be radially constrained at different locations by adhesion. In an example, a tubular mesh can be radially constrained at different locations by crimping or pinching.

In an example, a series of expandable meshes can comprise a (proximal to distal) stack of expandable meshes. In an example, the central axes of expandable meshes in a stack can be linearly aligned. In an example, at least one of the expandable meshes in the series can have a diameter which is greater than the diameter of a aneurysm neck. In an example, at least one of the expandable meshes in the series can have a diameter which is at least 25% greater than the diameter of a aneurysm neck.

In an example, a series of expandable meshes can comprise at least three expandable meshes: a proximal expandable mesh which is closest to the aneurysm neck after deployment; a distal expandable mesh which is farthest from the aneurysm neck after deployment; and a central expandable mesh which is between the proximal expandable mesh and the distal expandable mesh. In an example, the central expandable mesh can be at least 20% larger (e.g. wider) than either the proximal expandable mesh or the distal expandable mesh. In another example, the proximal expandable mesh can be at least 20% larger (e.g. wider) than either the central expandable mesh or the distal expandable mesh.

In an example, an expandable mesh with a longitudinal series of radial (e.g. radially-sinusoidal) undulations can comprise at least three undulations: a proximal undulation which is closest to the aneurysm neck after deployment; a distal undulation which is farthest from the aneurysm neck after deployment; and a central undulation which is between the proximal undulation and the distal undulation. In an example, radial undulation is repeated variation in radial distance of mesh walls as one travels along the central longitudinal axis of a mesh. In an example, the central undulation can be at least 20% larger (e.g. wider) than either the proximal undulation or the distal undulation. In another example, the proximal undulation can be at least 20% larger (e.g. wider) than either the central undulation or the distal undulation. Relevant variations discussed elsewhere in this disclosure or in priority-linked disclosures can also be applied to this example.

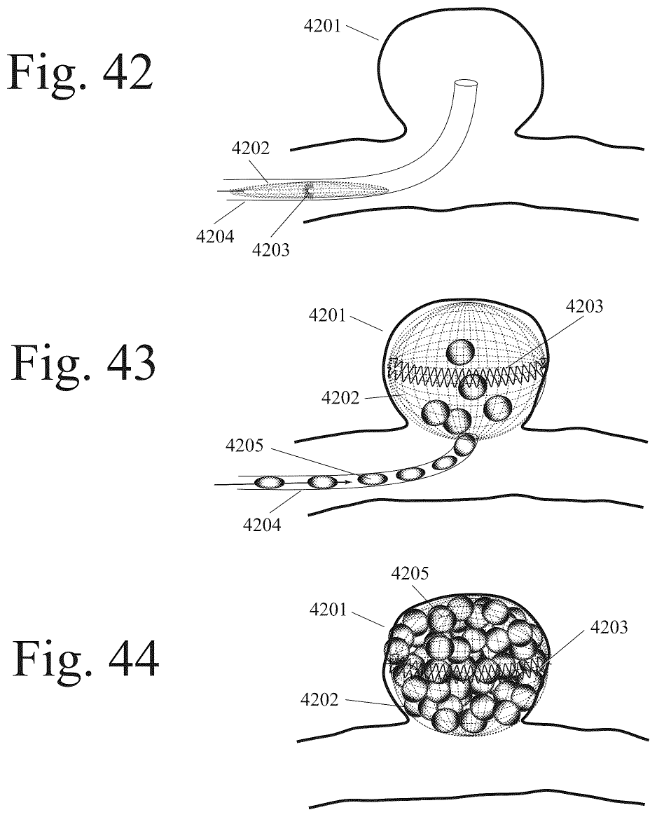

FIGS. 42 through 44 show three views, at three different times, of an intrasaccular aneurysm occlusion device comprising: a flexible net or mesh 4202 that is inserted into an aneurysm sac 4201; an expandable band or ring 4203 that is inserted into the aneurysm sac, wherein the expandable band or ring is around a circumference of the flexible net or mesh; embolic components or material (e.g. embolic beads, microspheres, microsponges, hydrogels, coils, string-of-pearls strands, or congealing liquid) 4205 which are inserted into the flexible net or mesh; and a catheter 4204 through which the flexible net or mesh, the expandable band or ring, and/or the embolic components or material are delivered to the aneurysm sac. In an example, this device can fill more of irregularly-shaped aneurysm sac than an expandable mesh with a specific preset (e.g. spherical, apple, or barrel) shape.

FIG. 42 shows this device at a first time, before the flexible net or mesh and the expandable band or ring have been inserted into the aneurysm sac. FIG. 43 shows this device at a second time, after the flexible net or mesh and the expandable band or ring have been inserted into the aneurysm sac, when embolic components and/or material are being inserted into the net or mesh. FIG. 44 shows this device at a third time, after embolic components and/or material have been inserted into the flexible net or mesh and the catheter has been removed.

In an example, the expandable band or ring can be around a longitudinally-central circumference of the flexible net or mesh. In an example, the expandable band or ring can be connected and/or attached to a longitudinally-central circumference of the flexible net or mesh. In an example, the expandable band or ring can be on the outside of a longitudinally-central circumference of the flexible net or mesh. In an example, the expandable band or ring can be on the inside of a longitudinally-central circumference of the flexible net or mesh. When the expandable band or ring is around a central circumference of the flexible net or mesh, this device can be described as having a “Saturn” design and/or shape.

In an example, the expandable band or ring can be a resilient mesh which self-expands radially after it exits from the catheter. In an example, the expandable band or ring can comprise a braid or weave of metal wires or tubes. In an example, the expandable band or ring can help to keep the flexible net or mesh from slipping out from the aneurysm sac. In an example, friction between the expandable band or ring and the aneurysm sac walls can help to keep the flexible net or mesh within the aneurysm sac.

In an example, the expandable band or ring can self-expand to a diameter which is at least 80% of the maximum and/or central diameter of the aneurysm sac. In an example, the expandable band or ring can self-expand to a diameter which is at least 25% greater than the diameter of the aneurysm neck. In an example, the expandable band or ring can have a width which is between 5% and 20% of the height of aneurysm sac (e.g. from the aneurysm neck to the aneurysm dome).

In an example, a flexible net or mesh can be made from a polymer. In an example, a flexible net or mesh can be made from a thermoelastic polymer. In an example, a flexible net or mesh can be formed by braiding or weaving polymer strands. In an example, a flexible net or mesh can be made by 3D printing. In an example, a flexible net or mesh can be made by inverting or everting a mesh tube. In an example, a flexible net or mesh can be made by cutting holes in a globular polymer surface (e.g. a balloon) with a laser.

In an example, a flexible net or mesh can be elastic and or stretchable. In an example, a flexible net or mesh can be made from a thermoplastic polymer. In an example, a distal portion (e.g. distal half or third) of a flexible net or mesh can be more porous than a proximal portion (e.g. proximal half or third) of the flexible net or mesh. In an example, a distal portion (e.g. distal half or third) of a flexible net or mesh can be more elastic and or stretchable than a proximal portion (e.g. proximal half or third) of the flexible net or mesh. In an example, a flexible net or mesh can be a hexagonal (or “honeycomb”) mesh (e.g. with hexagonal pores or openings). In an example, the flexible net or mesh can be folded and or pleated as it travels through the catheter to the aneurysm sac.

In an example, a flexible net or mesh can be convex after it is expanded by insertion of embolic components or material (e.g. embolic beads, microspheres, microsponges, hydrogels, coils, string-of-pearls strands, or congealing liquid). In an example, a flexible net or mesh can be irregularly shaped (to conform to the walls of an irregular aneurysm sac) after the net or mesh is expanded by insertion of embolic components or material. In an example, insertion of embolic components and/or material into the flexible net or mesh can cause the flexible net or mesh to expand in a manner which fills even an irregularly-shaped aneurysm sac.

In an example, a flexible net or mesh can be made from flexible metal wires or tubes. In an example, a flexible net or mesh can be formed by braiding or weaving flexible metal wires or tubes. In an example, a flexible net or mesh can be made from a combination of metal wires (or tubes) and polymer strands. In an example, a flexible net or mesh can be formed by weaving or braiding metal wires (or tubes) and polymer strands together. In an example, a flexible net or mesh can be made from a combination of metal wires (or tubes) and thermoelastic polymer strands.

In an example, there can be an opening (e.g. hole) on the proximal side (or surface) of the flexible net or mesh through which embolic components and/or material can be inserted into the flexible net or mesh. In an example, embolic components and/or material can be pushed into a flexible net or mesh by a flowable substance (e.g. saline) which flows through the catheter. In an example, a flowable substance can escape from the aneurysm sac through the flexible net or mesh, but embolic components and/or material are retained in the aneurysm sac because they does not pass through the flexible net or mesh.

In an example, there can be an opening on the proximal side (or surface) of the flexible net or mesh through which embolic components and or material can be inserted into the net or mesh, wherein there is also a closure mechanism which a device operator can control remotely to selectively open or close the valve opening. In an example, this closure mechanism can be selected from the group consisting of: adhesive, ball valve, butterfly valve, cap, check valves, clamp, clip, diaphragm valve, drawstring, fusible member, gate value, globe valve, loop, magnet, pinch valve, plug, plug valve, pneumatic valve, pull cord, pull wire, seal, sliding valve, snap, solenoid, and threaded valve.

In an example, embolic components and/or material can comprise embolic beads, microspheres, or microsponges. In an example, embolic components and/or material can comprise hydrogels. In an example, embolic components and/or material can comprise metal coils. In an example, embolic components and/or material can comprise polymer coils, strands, or ribbons. In an example, embolic components and/or material can comprise string-of-pearls strands (e.g. longitudinal series of embolic pieces connected by wires, sutures, strings, cords, springs, or coils). In an example, embolic components and/or material can comprise a liquid or gel which congeals after insertion into an aneurysm sac.

In an example, embolic components and/or material can be conveyed through a catheter by pusher wire and/or plunger. In an example, embolic components and/or material can be conveyed through a catheter by a liquid flow. In an example, embolic components and/or material can be conveyed through a catheter by a moving conveyor belt and/or wire loop. In an example, embolic components and/or material can be conveyed through a catheter by a rotating helix (e.g. Archimedes screw). Relevant variations discussed elsewhere in this disclosure or in priority-linked disclosures can also be applied to this example.

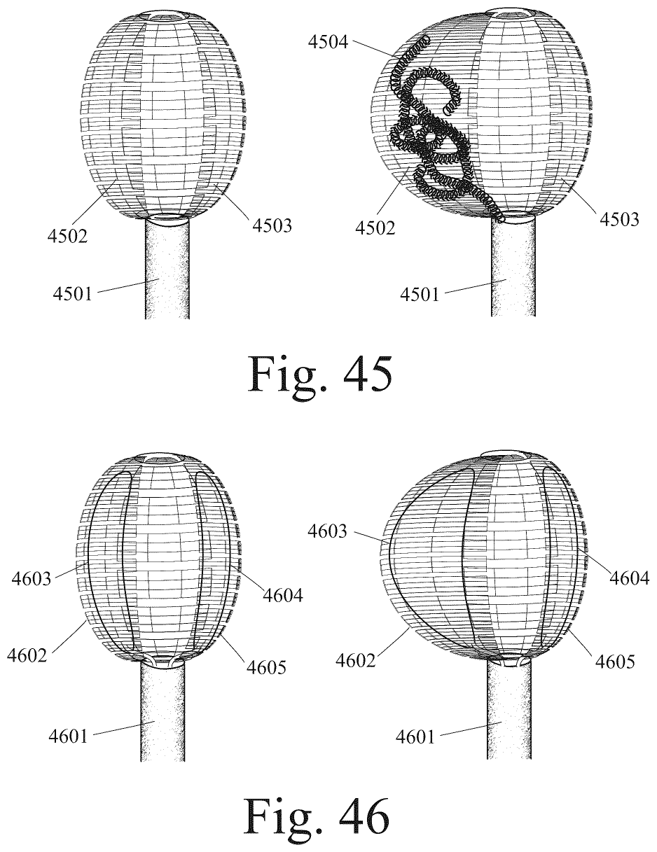

FIG. 45 shows two views, at two different times, of an intrasaccular aneurysm occlusion device comprising: a convex expandable mesh (e.g. mesh, lattice, braid, stent, and/or neck bridge) that is inserted and expanded within an aneurysm sac, wherein the convex expandable mesh further comprises a plurality of (proximal-to-distal) chambers (including 4502 and 4503) within the convex expandable mesh, wherein the chambers intersect the circumference of the mesh at different locations; embolic components and/or material 4504, wherein different amounts of the embolic components and/or material are inserted into different chambers, thereby expanding the convex expandable mesh in a radially-asymmetric manner and/or steering the mesh in a selected direction, at a selected angle, or with a selected orientation; and a catheter 4501 which delivers the convex expandable mesh to the aneurysm sac.

In an example, a device operator can control radially-asymmetric expansion of the mesh and/or steer the mesh in a selected direction by inserting different amounts of embolic components and/or material into different chambers in the mesh. In the example shown in FIG. 45, embolic components and/or material has been inserted into chamber 4502, but not chamber 4503, thereby expanding the mesh in a radially-asymmetric manner. The left portion of FIG. 45 shows this device at a first time, before embolic components and/or material has been inserted into any chamber. The right portion of FIG. 45 shows this device at a second time, when embolic components and/or material have been inserted into chamber 4502, but not chamber 4503.

In an example, there can be two (proximal-to-distal) chambers in a convex expandable mesh, one on each side (e.g. on opposite sides) of the mesh. In an example, there can be three or more chambers in a convex expandable mesh, wherein these chambers are evenly distributed around the circumference of the mesh. In an example, embolic components and/or material which are inserted into chambers can comprise embolic beads, microspheres, or microsponges. In an example, embolic components and/or material can comprise hydrogels. In an example, embolic components and/or material can comprise metal coils. In an example, embolic components and/or material can comprise polymer coils, strands, or ribbons. In an example, embolic components and/or material can comprise string-of-pearls strands (e.g. longitudinal series of embolic pieces connected by wires, sutures, strings, cords, springs, or coils). Relevant variations discussed elsewhere in this disclosure or in priority-linked disclosures can also be applied to this example.

FIG. 46 shows two views, at two different times, of an intrasaccular aneurysm occlusion device comprising: a convex expandable mesh (e.g. mesh, lattice, braid, stent, and/or neck bridge) that is inserted and expanded within an aneurysm sac, wherein the convex expandable mesh further comprises a plurality of (proximal-to-distal) chambers (4602 and 4605) within the convex expandable mesh; a plurality of expandable components (4603 and 4604) within the chambers, wherein the expandable components are expanded by inserting a flowable substance (e.g. a fluid, gas, or gel) into them, and wherein different amounts of the flowable substance are inserted into different expandable components, thereby expanding the convex expandable mesh in a radially-asymmetric manner and/or steering the mesh in a selected direction, at a selected angle, or with a selected orientation; and a catheter 4601 which delivers the convex expandable mesh to the aneurysm sac.

In an example, a device operator can control radially-asymmetric expansion of the mesh and/or steer the mesh in a selected direction by inserting different amounts of flowable substance into different expandable components in the mesh. In the example shown in FIG. 46, flowable substance has been inserted into expandable component 4603, but not expandable component 4604, thereby expanding the mesh in a radially-asymmetric manner. The left portion of FIG. 46 shows this device at a first time, before flowable substance has been inserted into any expandable component. The right portion of FIG. 46 shows this device at a second time, when flowable substance have been inserted into expandable component 4603, but not component 4604.

In an example, there can be two expandable components in a convex expandable mesh, one on each side (e.g. on opposite sides) of the mesh. In an example, there can be three or more expandable components in a convex expandable mesh, wherein these chambers are evenly distributed around the circumference of the mesh. In an example, a flowable substance which is inserted into expandable components can be a gas, liquid, or gel. Relevant variations discussed elsewhere in this disclosure or in priority-linked disclosures can also be applied to this example.

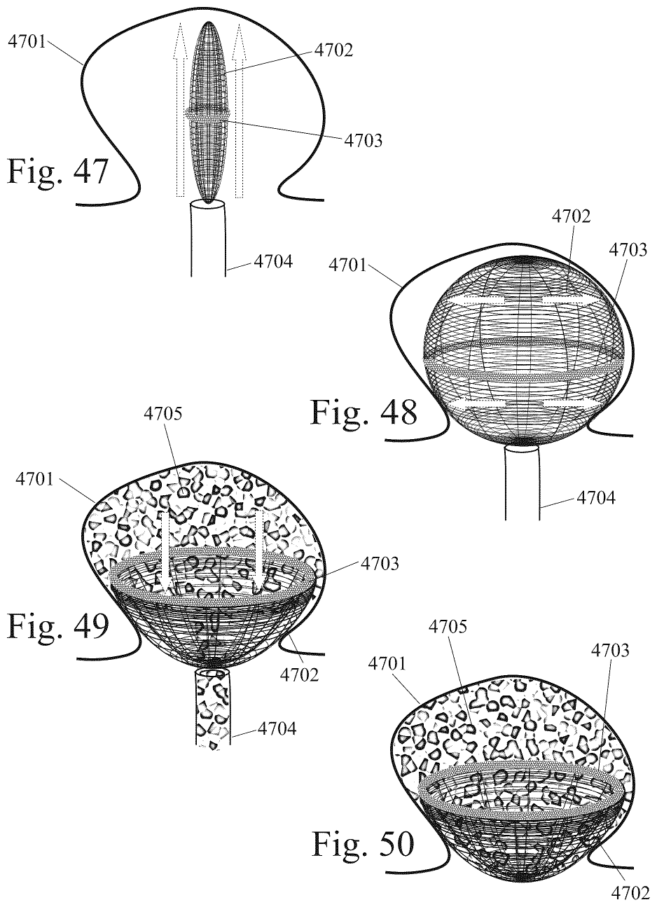

FIGS. 47 through 50 shows four views, at four different times, of an intrasaccular aneurysm occlusion device comprising: (a) an expandable mesh (e.g. mesh, lattice, braid, stent, and/or neck bridge) 4702 that is inserted and expanded within an aneurysm sac 4701, wherein the expandable mesh is expanded in a radial manner at a first time within the aneurysm sac into a first configuration having a convex (e.g. globular, spherical, ellipsoidal, or oblate spheroidal) shape; wherein the expandable mesh is compressed in a distal-to-proximal manner at a second time within the aneurysm sac into a second configuration having a concave (e.g. bowl, cup, hemispherical, hemi-ellipsoidal, or funnel) shape; wherein the expandable mesh further comprises a (central) annular section (or band) 4703 which is more elastic and/or flexible than the rest of the expandable mesh; wherein a first portion (e.g. first third or half) of the expandable mesh is distal relative to the annular section (or band) in the first configuration; wherein a second portion (e.g. second third or half) of the expandable mesh is proximal relative to the annular section (or band) in the first configuration; wherein the first portion of the expandable mesh is compressed, inverted, and/or nested into the second portion of the expandable mesh as the expandable mesh is changed from the first configuration to the second configuration; wherein both the first portion of the expandable mesh and the second portion of the expandable mesh are proximal relative to the annular section (or band) in the second configuration; (b) embolic components and/or material (e.g. beads, microspheres, microsponges, hydrogels, congealing liquid or gel, or embolic coils) 4705 that are inserted into portions of the aneurysm sac which are distal to the expandable mesh, and wherein insertion of the embolic components and/or material into the aneurysm sac compresses (e.g. pressures or compels) the expandable mesh from the first configuration having the convex shape to the second configuration having the concave shape; and (c) one or more catheters 4704 which deliver the expandable mesh and/or the embolic components and/or material to the aneurysm sac.

FIG. 47 shows this device after the expandable mesh has been inserted into an aneurysm sac, but before the mesh has been expanded in a radial manner. FIG. 48 shows this device after the expandable mesh has expanded in a radial manner into its convex first configuration. FIG. 49 shows this device as embolic members and/or material are being inserted into the aneurysm sac in a location which is distal to the expandable mesh, thereby compressing the expandable mesh from its convex first configuration into its concave second configuration. FIG. 50 shows this device after embolic members and/or material have been inserted, wherein the expandable mesh is now completely in its concave second configuration and the catheter has been removed.

In an example, a distal portion (e.g. distal third or half) of a convex expandable mesh can be collapsed, compressed, folded, and/or inverted into a concavity of a proximal portion (e.g. proximal third or half) of the expandable mesh within an aneurysm sac. In an example, a distal portion (e.g. distal third or half) of a convex expandable mesh can be collapsed, compressed, folded, and/or inverted along an flexible annular section (or band) of the mesh into a concavity of a proximal portion (e.g. proximal third or half) of the expandable mesh.

In an example, an expandable mesh which is inserted into an aneurysm can have a first configuration and a second configuration, wherein the first configuration is convex (e.g. globular) and the second configuration is concave (e.g. bowl shaped), wherein the mesh expands into the first configuration in an aneurysm sac after it exits a catheter, and wherein the mesh is collapsed, compressed, folded, and/or inverted into the second configuration after expanding into the first configuration. In an example, when an expandable mesh is in a convex (e.g. globular) configuration, the expandable mesh can comprise a distal portion (e.g. distal third or third or half), a proximal portion (e.g. proximal third or third or half), and flexible annular section (or band) around a (central) circumference between the distal portion and the proximal portion, wherein the distal portion is collapsed, compressed, and/or inverted into a concavity of the proximal portion.

In an example, an expandable mesh can expand into a globular (e.g. spherical, ellipsoidal, or oblate spheroidal) shape in an aneurysm sac and then be collapsed, compressed, and/or inverted into a concave (e.g. bowl, cup, hemispherical, hemi-ellipsoidal) shape by pressure from the accumulation of embolic members and/or material in a distal portion of the aneurysm sac. In an example, an expandable mesh can be changed from a convex (e.g. globular) shape to a concave (e.g. bowl) shape by collapsing, compressing, and/or inverting a distal portion of the mesh into a concavity of a proximal portion of the mesh by pressure on the distal portion of the mesh from accumulation of embolic members and/or material in an aneurysm sac.

In an example, a distal portion (e.g. distal third or half) of a convex expandable mesh can be collapsed, compressed, and/or inverted along an elastic and/or flexible annular section (or band) of the mesh into a concavity of a proximal portion (e.g. proximal third or half) of the expandable mesh by the insertion of embolic members and/or material into an area of an aneurysm sac which is distal relative to the expandable mesh. In an example, when an expandable mesh is in a convex configuration, the expandable mesh can comprise a distal portion (e.g. distal third or third or half), a proximal portion (e.g. proximal third or third or half), a flexible annular section (or band) around a (central) circumference between the distal portion and the proximal portion, and a wire or cord attached to the distal portion, wherein the distal portion is collapsed, compressed, and/or inverted into a concavity of the proximal portion by pressure from the accumulation of embolic members and/or material in (a distal area of) an aneurysm sac.

In an example, a first portion of an expandable mesh can be distal to a second portion of the expandable mesh in a first configuration, but the first portion can be nested within a concavity of the second portion in a first configuration after the expandable mesh has been collapsed, compressed, and/or inverted in a distal-to-proximal direction. In an example, an expandable mesh can have a single-layer convex first configuration and a two-layer concave second configuration, wherein the expandable mesh has a distal portion (e.g. distal third or half) and a proximal portion (e.g. proximal third or half) in the convex first configuration, wherein the distal portion is collapsed, compressed, and/or inverted into the proximal portion in the second configuration, and wherein the distal portion is nested within a concavity of the proximal portion in the second configuration.

In an example, an expandable mesh which is inserted into an aneurysm sac can comprise: a first portion (e.g. first third or half) which is a distal portion when the mesh is in an expanded convex configuration; a second portion (e.g. second third or half) which is a proximal portion when the mesh is in the expanded convex configuration; and a flexible section between the first portion and the second portion, wherein the flexible section spans a (central) perimeter of the expandable mesh, and wherein the first portion is collapsed, compressed, and/or inverted around the flexible section into a concavity of the second portion.

In an example, an expandable mesh can comprise a first concave portion which opens proximally, a second concave portion which opens distally, and an annular section (or band) between the first and second concave portions, wherein the expandable mesh has a first configuration in which the first concave portion is distal relative to the annular section (or band) and the second concave portion is proximal relative to the annular section (or band), and wherein the expandable mesh has a second configuration in which the first concave portion is inverted into the second concave portion and both the first and second concave portions are proximal relative to the annular section (or band).

In an example, an expandable mesh can be changed from a convex (e.g. globular) shape to a concave (e.g. bowl) shape by collapsing, compressing, and/or inverting a distal portion of the mesh into a concavity of a proximal portion of the mesh by pulling a wire attached to the distal portion. In an example, when an expandable mesh is in a convex configuration, the expandable mesh can comprise a distal portion (e.g. distal third or third or half), a proximal portion (e.g. proximal third or third or half), a flexible annular section (or band) around a (central) circumference between the distal portion and the proximal portion, and a wire or cord attached to the distal portion, wherein the distal portion is collapsed, compressed, and/or inverted into a concavity of the proximal portion when the wire or cord is pulled in a proximal direction.

In an example, when an expandable mesh is in a convex configuration, the expandable mesh can comprise a distal portion (e.g. distal third or third or half), a proximal portion (e.g. proximal third or third or half), a flexible annular section (or band) around a (central) circumference between the distal portion and the proximal portion, and a wire attached to the distal portion, wherein the distal portion is collapsed, compressed, and/or inverted into a concavity of the proximal portion when the wire or cord is pulled by a device operator.

In an example, an expandable mesh can expand into a globular (e.g. spherical, ellipsoidal, or oblate spheroidal) shape in an aneurysm sac and then be collapsed, compressed, and/or inverted into a concave (e.g. bowl, cup, hemispherical, hemi-ellipsoidal) shape. In an example, an expandable mesh can have a first convex (e.g. globular, spherical, ellipsoidal, or oblate spheroidal) shape and a second concave (e.g. bowl, cup, hemispherical, or funnel) shape, wherein the mesh is collapsed, compressed, and/or inverted from the first shape to the second shape by pressure from accumulation of embolic members and/or material in an aneurysm sac. In an example, an expandable mesh can have a first convex (e.g. globular, spherical, ellipsoidal, or oblate spheroidal) shape and a second concave (e.g. bowl, cup, hemispherical, or funnel) shape, wherein the mesh is collapsed, compressed, and/or inverted from the first shape to the second shape by pulling a wire or cord attached to a distal portion of the mesh.

In an example, an expandable mesh can be made by braiding or weaving metal wires or tubes. In an example, an expandable mesh can be formed by braiding or weaving polymer strands. In an example, an expandable mesh can be formed by weaving or braiding metal wires (or tubes) and polymer strands together. In an example, an expandable mesh can be made by 3D printing. In an example, an expandable mesh can radially self-expand within an aneurysm sac. In an example, an expandable mesh can have a globular and/or spherical shape in a first configuration. In an example, an expandable mesh can have an ellipsoidal or oblate spheroidal shape in a first configuration. In an example, an expandable mesh can have a half-torus shape in a first configuration.

In an example, an expandable mesh can have a concave bowl or cup shape in a second configuration. In an example, an expandable mesh can have a funnel or half-hyperbolic shape in a second configuration. In an example, an expandable mesh can have a hemispherical or hemi-ellipsoidal concave shape in a second configuration. In an example, expandable mesh can be expanded in a radially-asymmetric manner by a device operator in order to steer it in a selected direction, at a selected angle, or with a selected orientation. In an example, a single-layer distal portion (e.g. distal third or half) of a convex expandable mesh can be compressed into a concavity of a proximal portion (e.g. proximal third or half) of the expandable mesh to form a two-layer convex expandable mesh within an aneurysm sac.

In an example, an expandable mesh can comprise a first set of wires, tubes, strands, or filaments with a first orientation (e.g. a circumferential orientation) and a second set of wires, tubes, strands, or filaments with a second orientation (e.g. a proximal-to-distal orientation), wherein wires, tubes, strands, or filaments in the first set are thinner or more sparsely configured in a flexible annular section (or band) of the expandable mesh than in the rest of the expandable mesh (e.g. in distal and proximal portions of the annular mesh). In an example, an expandable mesh can comprise a first set of wires, tubes, strands, or filaments with a first orientation (e.g. a circumferential orientation) and a second set of wires, tubes, strands, or filaments with a second orientation (e.g. a proximal-to-distal orientation), wherein wires, tubes, strands, or filaments in the second set are thinner or more sparsely configured in a flexible annular section (or band) of the expandable mesh than in the rest of the expandable mesh (e.g. in distal and proximal portions of the annular mesh).

In an example, a flexible annular section (or band) around a circumference of an expandable mesh can span a central circumference of the expandable mesh. In an example, a flexible section of an expandable mesh can be around a central perimeter of the expandable mesh. In an example, an expandable band or ring can be around a central circumference of an expandable mesh. In an example, a flexible annular section (or band) around a circumference of an expandable mesh can span a widest circumference of the expandable mesh.

In an example, an expandable mesh which is inserted into an aneurysm sac can comprise: a first portion (e.g. first third or half) which is a distal portion when the mesh is in an expanded convex configuration; a second portion (e.g. second third or half) which is a proximal portion when the mesh is in the expanded convex configuration; and a flexible section between the first portion and the second portion, wherein the flexible section spans a (central) perimeter of the expandable mesh.

In an example, a flexible annular section (or band) of an expandable mesh can have a circular cross-sectional shape. In an example, a flexible annular section (or band) of an expandable mesh can be circumferential. In an example, a cross-section of a flexible annular section (or band) of an expandable mesh can have a convex shape. In an example, a flexible annular section (or band) of an expandable mesh can have an elliptical cross-sectional shape. In an example, a cross-section of a flexible annular section (or band) of an expandable mesh can have an undulating (e.g. sinusoidal) circular shape. In an example, a cross-section of a flexible annular section (or band) of an expandable mesh can have an undulating (e.g. sinusoidal) convex shape. In an example, a flexible annular section (or band) of an expandable mesh can have an undulating (e.g. sinusoidal) cross-sectional shape.

In an example, a flexible annular section (or band) around a (central) perimeter of an expandable mesh can have a variable-width around its circumference. In an example, a flexible annular section (or band) of an expandable mesh can have a uniform proximal-to-distal width. In an example, a flexible annular section (or band) around a (central) perimeter of an expandable mesh can be radially asymmetric. In an example, a flexible annular section (or band) around a (central) circumference of an expandable mesh can have a proximal-to-distal width which is less than 2 mm. In an example, a flexible annular section (or band) around a (central) circumference of an expandable mesh can have a proximal-to-distal width between 1 mm and 3 mm. In an example, a flexible annular section (or band) around a (central) circumference of an expandable mesh can have a proximal-to-distal width between 2 mm and 5 mm.

In an example, a proximal-to-distal width of an annular section (or band) of an expandable mesh can be between 5% and 15% of the length of a proximal-to-distal axis of the mesh when the mesh is in a convex first configuration. In an example, a proximal-to-distal width of an annular section (or band) of an expandable mesh can be between 10% and 25% of the length of a proximal-to-distal axis of the mesh when the mesh is in a convex first configuration. In an example, an annular section (or band) around a (central) circumference of an expandable mesh can comprise between 2% and 10% of the (surface area of the) expandable mesh. In an example, an annular section (or band) around a (central) circumference of an expandable mesh can comprise between 5% and 20% of the (surface area of the) expandable mesh.

In an example, an elastic and/or flexible annular section (or band) around the central circumference of an expandable mesh can comprise between 5% and 20% of the surface area of the expandable mesh. In an example, an elastic and/or flexible annular section (or band) can comprise around the central circumference of an expandable mesh can comprise between 5% and 20% of the height of aneurysm sac (e.g. from the aneurysm neck to the aneurysm dome). In an example, an expandable mesh which is inserted into an aneurysm can comprise: a distal portion of an expandable mesh, a proximal portion of the expandable mesh, and a flexible annular portion of the expandable mesh, wherein the flexible annular portion is between the distal portion and the proximal portion, wherein the distal portion spans a first percentage (e.g. between 25% and 50%) of the proximal-to-distal axis of the expandable mesh when the mesh is in an expanded convex configuration, wherein the proximal portion spans a second percentage (e.g. between 25% and 50%) of the proximal-to-distal axis of the expandable mesh when the mesh is in the expanded convex configuration, and wherein the flexible annular portion spans the remaining percentage (e.g. 100% minus the first and second percentages) of the proximal-to-distal axis.

In an example, an expandable mesh which is inserted into an aneurysm can comprise: a distal portion of an expandable mesh, a proximal portion of the expandable mesh, and a flexible annular portion of the expandable mesh, wherein the flexible annular portion is between the distal portion and the proximal portion, wherein the distal portion spans a first percentage (e.g. between 30% and 45%) of the proximal-to-distal axis of the expandable mesh when the mesh is in an expanded convex configuration, wherein the proximal portion spans a second percentage (e.g. between 30% and 45%) of the proximal-to-distal axis of the expandable mesh when the mesh is in the expanded convex configuration, and wherein the flexible annular portion spans the remaining percentage (e.g. 100% minus the first and second percentages) of the proximal-to-distal axis.

In an example, an annular section (or band) around a central circumference of an expandable mesh can be more flexible than the rest of the mesh. In an example, a flexible annular section (or band) around a (central) circumference of an expandable mesh can be more elastic than distal or proximal portions of the expandable mesh. In an example, a flexible annular section (or band) around a (central) circumference of an expandable mesh can be more elastic than the rest of the expandable mesh. In an example, a flexible annular section (or band) around a (central) circumference of an expandable mesh can be more elastic and/or stretchable than the rest of the expandable mesh. In an example, a flexible annular section (or band) around a (central) circumference of an expandable mesh can be more elastic and/or stretchable than distal or proximal portions of the expandable mesh.

In an example, an annular section (or band) around a central circumference of an expandable mesh can be more elastic than the rest of the mesh. In an example, an annular section (or band) of an expandable mesh can be made from material which is more elastic and/or more flexible than material used to make the rest of the expandable mesh. In an example, wires, tubes, or fibers comprising an annular section (or band) of an expandable mesh can be made with one or more materials which are more elastic and/or flexible than the one or more materials used to make the wires, tubes, or fibers comprising the rest of the expandable mesh.

In an example, a flexible annular section (or band) around a (central) circumference of an expandable mesh can be made with lower durometer material than the rest of the expandable mesh. In an example, a flexible annular section (or band) around a (central) circumference of an expandable mesh can be made with lower durometer material than distal or proximal portions of the expandable mesh. In an example, an annular section (or band) around a central circumference of an expandable mesh can have a lower durometer and/or Shore value than the rest of the mesh. In an example, a flexible annular section (or band) around a (central) circumference of an expandable mesh can have a less dense mesh, weave, or braid than the rest of the expandable mesh. In an example, a flexible annular section (or band) around a (central) circumference of an expandable mesh can have a less dense mesh, weave, or braid than distal or proximal portions of the expandable mesh.

In an example, a flexible annular section (or band) around a (central) circumference of an expandable mesh can be thinner (and/or have thinner mesh components) than the rest of the expandable mesh. In an example, a flexible annular section (or band) around a (central) circumference of an expandable mesh can be thinner (and/or have thinner mesh components) than distal or proximal portions of the expandable mesh. In an example, wires, tubes, or fibers comprising an annular section (or band) of an expandable mesh can be thinner and/or smaller than wires, tubes, or fibers comprising the rest of the expandable mesh.

In an example, an expandable mesh can comprise a weave or braid of a first set of wires, tubes, filaments, or strands with a first orientation and a second set of wires, tube, filaments, or strands with a second orientation, wherein the first and second sets of wires, tubes, filaments, or strands intersect to form the weave or braid, and wherein a flexible annular section (or band) around a (central) circumference of the expandable mesh comprises only one of the sets of wires, tubes, filaments, or strands. In an example, an expandable mesh can comprise a weave or braid of a first set of wires, tubes, filaments, or strands with a first orientation and a second set of wires, tube, filaments, or strands with a second orientation, wherein the first and second sets of wires, tubes, filaments, or strands intersect to form the weave or braid, and wherein a flexible annular section (or band) around a (central) circumference of the expandable mesh has a different ratio, proportion, and/or blend of the first and second sets than the rest of the expandable mesh.

In an example, an expandable mesh can comprise a weave or braid of a first set of wires, tubes, filaments, or strands with a circumferential shape or orientation and a second set of wires, tube, filaments, or strands with a longitudinal shape or orientation, wherein the first and second sets of wires, tubes, filaments, or strands intersect to form the weave or braid, and wherein a flexible annular section (or band) around a (central) circumference of the expandable mesh has a different ratio, proportion, and/or blend of the first and second sets than the rest of the expandable mesh.

In an example, braid or weave of wires, tubes, or fibers comprising an annular section (or band) of an expandable mesh can be more sparse, less dense, more open, and/or more loose than a braid or weave of wires, tubes, or fibers comprising the rest of the expandable mesh. In an example, the braid or weave of wires, tubes, or fibers comprising an annular section (or band) of an expandable mesh can be more sparse, less dense, more open, and/or more loose than that of the rest of the mesh. In an example, wires, tubes, and/or strands used to form an annular section (or band) around a central circumference of an expandable mesh can be more flexible, more elastic, thinner, smaller, and/or woven less densely than those in rest of the mesh.

In an example, an expandable mesh can comprise an annular section (or band) around a (central) conference of the mesh, wherein the annular section (or band) is more flexible, more elastic, and/or thinner than the rest of the mesh so that a distal portion (e.g. distal third or half) of the collapses, compresses, and/or inverts into a proximal portion (e.g. proximal third or half) of the mesh when pressure is applied to the distal portion. In an example, an expandable mesh can comprise an annular section (or band) around a (central) conference of the mesh, wherein the annular section (or band) is more flexible, more elastic, and/or thinner than the rest of the mesh so that a distal portion (e.g. distal third or half) of the mesh collapses, compresses, and/or inverts into a proximal portion (e.g. proximal third or half) of the mesh when pressure is applied to the distal portion by the accumulation of embolic members and/or embolic material in an aneurysm sac.

In an example, an expandable mesh which is inserted into an aneurysm sac can comprise: a distal portion (e.g. distal third) of the expandable mesh; a proximal portion (e.g. proximal third) of the expandable mesh; and a central portion (e.g. central third) of the expandable mesh; wherein the central portion is annular (e.g. around the central circumference of the expandable mesh); wherein the central portion is more flexible, more elastic, less dense, and/or thinner than the distal and proximal portions; and wherein the distal portion is collapsed, compressed, and/or inverted into a concavity of the proximal portion by pressure from an accumulation of embolic members and/or material in the aneurysm sac. In an example, an expandable mesh which is inserted into an aneurysm sac can comprise: a distal portion (e.g. distal third) of the expandable mesh; a proximal portion (e.g. proximal third) of the expandable mesh; and a central portion (e.g. central third) of the expandable mesh; wherein the central portion is annular (e.g. around the central circumference of the expandable mesh); wherein the central portion is more flexible, more elastic, less dense, and/or thinner than the distal and proximal portions; and wherein the distal portion is collapsed, compressed, and/or inverted into a concavity of the proximal portion.

In an example, a flexible annular section (or band) around a (central) circumference of an expandable mesh can be weakened remotely by a device operator. In an example, a flexible annular section (or band) around a (central) circumference of an expandable mesh can be weakened remotely by a device operator by transmission of electrical energy to the annular section (or band). In an example, selected components of a flexible annular section (or band) around a (central) circumference of an expandable mesh can be weakened (e.g. melted and/or detached) remotely by transmission of electrical energy. In an example, selected components of a flexible annular section (or band) around a (central) circumference of an expandable mesh can be weakened (e.g. melted and/or detached) remotely by exposure to light energy. In an example, selected components of a flexible annular section (or band) around a (central) circumference of an expandable mesh can be weakened (e.g. melted and/or detached) remotely by sonic energy.

In an example, embolic components and/or material can be inserted into an aneurysm sac through one or more openings in an expandable mesh. In an example, there can be one or more openings (e.g. holes or valves) in an expandable mesh through which embolic members and/or material are inserted to the aneurysm sac. In an example, embolic members and/or material can be inserted through an opening in an expandable mesh into the dome of an aneurysm sac, wherein pressure from the accumulation of the embolic members and/or material collapses, compresses, and/or inverts a distal portion (e.g. distal third or half) of the mesh into a concavity of a proximal portion of the mesh. In an example, embolic members and/or material can be inserted through a funnel-shaped opening in an expandable mesh into the dome of an aneurysm sac, wherein pressure from the accumulation of the embolic members and/or material collapses, compresses, and/or inverts a distal portion (e.g. distal third or half) of the mesh into a concavity of a proximal portion of the mesh,

In an example, an opening valve and/or closure mechanism can be selected from the group consisting of: adhesive, ball valve, butterfly valve, cap, check valves, clamp, clip, diaphragm valve, drawstring, fusible member, gate value, globe valve, loop, magnet, pinch valve, plug, plug valve, pneumatic valve, pull cord, pull wire, seal, sliding valve, snap, solenoid, and threaded valve. In an example, embolic members and/or material can be inserted through an opening in an expandable mesh into the dome of an aneurysm sac, wherein pressure from the accumulation of the embolic members and/or material collapses, compresses, and/or inverts a distal portion (e.g. distal third or half) of the mesh into a concavity of a proximal portion of the mesh, wherein there is a one-way valve in the opening.

In an example, embolic members and/or material can be inserted through an opening in an expandable mesh into the dome of an aneurysm sac, wherein pressure from the accumulation of the embolic members and/or material collapses, compresses, and/or inverts a distal portion (e.g. distal third or half) of the mesh into a concavity of a proximal portion of the mesh, wherein there is a leaflet (e.g. tri-leaflet) valve in the opening. In an example, embolic members and/or material can be inserted through an opening in an expandable mesh into the dome of an aneurysm sac, wherein pressure from the accumulation of the embolic members and/or material collapses, compresses, and/or inverts a distal portion (e.g. distal third or half) of the mesh into a concavity of a proximal portion of the mesh, wherein there is a valve in the opening which the device operator can open or close remotely.

In an example, there can be an opening on a proximal side (or surface) of an expandable mesh through which embolic components and or material are inserted, wherein there is also a closure mechanism which a device operator can control remotely to selectively open or close the opening. In an example, embolic members and/or material can be inserted through a gap between an expandable mesh and a wall of an aneurysm sac. In an example, embolic members and/or material can be inserted past an expandable mesh into an aneurysm sac through a gap (e.g. space) between the expandable mesh and the wall of the aneurysm sac.

In an example, accumulation of embolic components and/or material in a portion of an aneurysm sac which is distal to an expandable mesh can pressure, push, compel, and/or collapse a distal portion (e.g. distal third or half) of the expandable mesh into a concavity of a proximal portion (e.g. proximal third or half) of the expandable mesh. In an example, accumulation of embolic components and/or material in a portion of an aneurysm sac which is distal to an expandable mesh can pressure, push, compel, and/or collapse a distal portion (e.g. distal third or half) of the expandable mesh from a single-layer convex shape into a two-layer concave shape.

In an example, embolic components and/or material (e.g. beads, microspheres, microsponges, hydrogels, congealing liquid or gel, or embolic coils) can be are inserted into portions of the aneurysm sac which are distal to an expandable mesh. In an example, embolic components and/or material can be congealing liquid or gel. In an example, embolic components and/or material can be liquid or gel which congeals (e.g. solidifies) after insertion into an aneurysm sac. In an example, embolic components and/or material can be microsponges or hydrogels. In an example, embolic components and/or material can comprise embolic beads, microspheres, or microsponges. In an example, embolic components and/or material can comprise metal coils.

In an example, embolic components and/or material can comprise polymer coils, strands, or ribbons. In an example, embolic components and/or material can comprise string-of-pearls strands (e.g. longitudinal series of embolic pieces connected by wires, sutures, strings, cords, springs, or coils). In an example, embolic components or material can be selected from the group consisting of: embolic beads, microspheres, microsponges, hydrogels, metal coils, polymer coils, ribbons, string-of-pearls strands, congealing liquid, and congealing gel.

In an example, embolic components and/or material can be conveyed through a catheter by a pusher wire and/or plunger. In an example, embolic components and/or material can be conveyed through a catheter by a liquid flow. In an example, embolic components and/or material can be conveyed through a catheter by a moving conveyor belt and/or wire loop. In an example, embolic components and/or material can be conveyed through a catheter by a rotating helix (e.g. Archimedes screw). Relevant variations discussed elsewhere in this disclosure or in priority-linked disclosures can also be applied to this example.

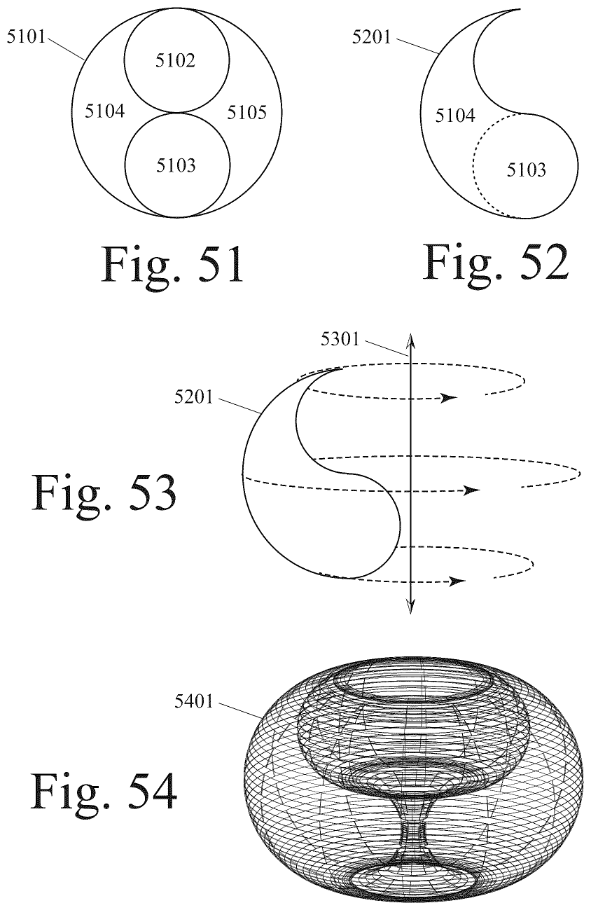

FIGS. 51 through 54 are a sequence of diagrams and figures which progressively show how an intrasaccular aneurysm occlusion device might be shaped like a three-dimensional revolution of a yin (or yang) symbol. This intrasaccular aneurysm occlusion device comprises an expandable mesh (e.g. mesh, lattice, braid, stent, and/or neck bridge) 5401 that is inserted and expanded within an aneurysm sac, wherein a shape of the expandable mesh is a shape formed by revolving the perimeter of a yin (or yang) symbol in three-dimensional space around an axis of revolution which coplanar with the yin (or yang) symbol and outside the perimeter of the yin (or yang) symbol. A yin (or yang) symbol is an arcuate half of the taijitu symbol.

The perimeter of a yin (or yang) symbol may be similar to the shape of a comma or single quote mark in some type fonts. However, there can be variation in this similarity due to variation in the geometries of different type fonts. The perimeter of a yin (or yang) symbol may not be universally understood and well-defined as a standard geometric shape. Accordingly, FIGS. 51 and 52 provide this geometric definition for the purposes of this disclosure. FIGS. 51 and 52 show how the perimeter of a yin (or yang) symbol can be formed by adding and subtracting areas of circles, which are standard geometric shapes. After the yin (or yang) symbol is formed, FIGS. 53 and 54 show how it can be revolved in three-dimensional space around an axis of revolution to get the shape of an expandable mesh for use as an intrasaccular aneurysm occlusion device.

FIG. 51 shows initial steps for creating a yin (or yang) symbol 5201 comprising: forming an outer circle 5101; forming a first inner circle 5102 and a second inner circle 5103 inside the outer circle, wherein diameters of the first and second inner circles are each half of the length of the diameter of the outer circle, and wherein diameters of the first and second inner circles are aligned along a diameter of the outer circle; identifying a first remaining area 5104 on a first side of the outer circle which is not spanned by either the first or second inner circles and a second remaining area 5105 on a second side of the outer circle which is not spanned by either the first or second inner circles. FIG. 52 shows the final step for creating the yin (or yang) symbol 5201. In this step, the area of the first inner circle 5102 and the second remaining area 5105 have been subtracted from the outer circle 5101. This leaves the area of the second inner circle 5103 and the first remaining area 5104. These two areas combine to form the perimeter of the yin (or yang) symbol 5201.

FIG. 53 shows diagrammatically how the perimeter of the yin (or yang) symbol formed in FIG. 52 can be revolved in three-dimensional space around an axis of revolution which is coplanar with the yin (or yang) symbol and outside the perimeter of the yin (or yang) symbol in order to create a three-dimensional surface. In an example, the axis of revolution can be parallel to the diameter of the outer circle with which the first and second inner circles were aligned. FIG. 54 shows a wire mesh representation of the three-dimensional surface created in FIG. 53. The expandable mesh in this example has this three-dimensional shape.

FIGS. 51 through 54 are useful for showing how the shape of this expandable mesh in this example can be geometrically specified and described, but do not necessarily show the method by which an actual expandable mesh is created. An expandable mesh with this three-dimensional shape need not be physically formed by revolving a yin (or yang) symbol. In an example, an expandable mesh with this three-dimensional shape can be formed by inverting, everting, and/or radially-constraining a tubular mesh multiple times. In an example, an expandable mesh with this three-dimensional shape can be made by 3D printing, weaving, braiding, or other methods. Relevant variations discussed elsewhere in this disclosure or in priority-linked disclosures can also be applied to this example.

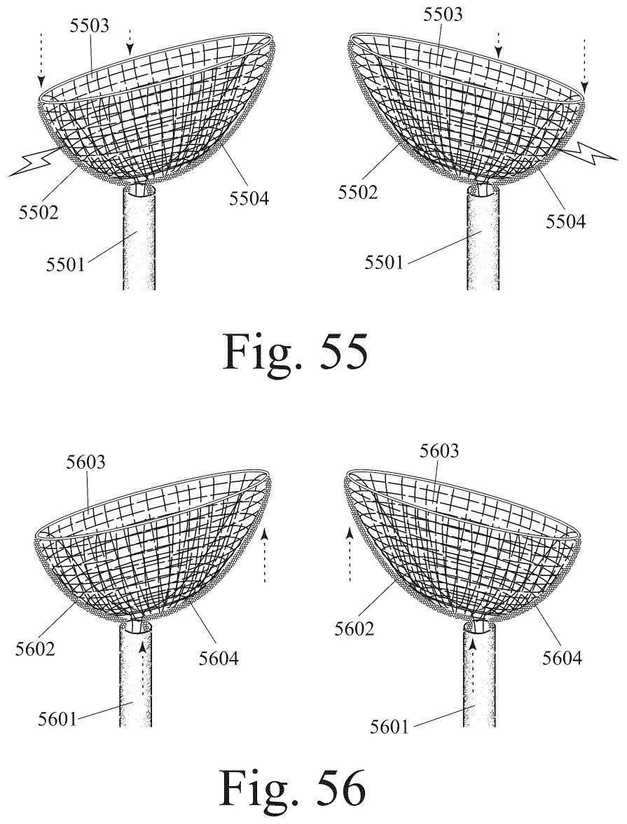

FIG. 55 shows two views, at two different times, of an intrasaccular aneurysm occlusion device comprising: an expandable mesh (e.g. mesh, lattice, braid, stent, and/or neck bridge) 5503 that is inserted and expanded within an aneurysm sac; a plurality of longitudinal piezoelectric members (e.g. cords or strands) 5502 and 5504 which are connected to different locations (e.g. to opposite sides) of the expandable mesh, wherein transmission of electricity to a selected subset of one or more of the piezoelectric members changes the lengths of the one or more piezoelectric members, causing the mesh to expand in an asymmetric manner and/or steering the mesh in a selected direction, at a selected angle, or with a selected orientation; and a catheter 5501 through which the mesh is delivered to the aneurysm sac.

The left portion of FIG. 55 shows this device at a first time, wherein electricity is transmitted to piezoelectric member (e.g. cord or strand) 5502. In this example, this transmission of electricity decreases the length of piezoelectric member 5502 and causes asymmetric expansion of the mesh. The right side of the mesh expands more than the left side of the mesh. The right portion of FIG. 55 shows this device at a second time, wherein electricity is transmitted to piezoelectric member (e.g. cord or strand) 5504. In this example, this transmission of electricity decreases the length of piezoelectric member 5504 and causes asymmetric expansion of the mesh. The left side of the mesh expands more than the right side of the mesh.

In an example, a device operator can control asymmetric expansion of the mesh by selectively transmitting electricity to a selected subset of one or more piezoelectric members (e.g. cords or strands) which are connected to different locations on the mesh. In an example, a device operator can steer the mesh in a selected direction, at a selected angle, or with a selected orientation by transmitting electricity to a selected subset of one or more piezoelectric members (e.g. cords or strands) which are connected to different locations on the mesh. In an example, there can be two piezoelectric members (e.g. cords or strands), one piezoelectric member connected to each side (e.g. opposite sides) of the expandable mesh. In an example, there can be three or more piezoelectric members (e.g. cords or strands) connected to different (e.g. equidistant) locations around the circumference of the expandable mesh. Relevant variations discussed elsewhere in this disclosure or in priority-linked disclosures can also be applied to this example.

FIG. 56 shows two views, at two different times, of an intrasaccular aneurysm occlusion device comprising: an expandable mesh (e.g. mesh, lattice, braid, stent, and/or neck bridge) 5603 that is inserted and expanded within an aneurysm sac; a plurality of wires 5602 and 5604 which are connected to different locations (e.g. to opposite sides) of the expandable mesh, wherein selective pulling or pushing one or more of the wires causes the mesh to expand in an asymmetric manner and/or steers the mesh in a selected direction, at a selected angle, or with a selected orientation; and a catheter 5601 through which the mesh is delivered to the aneurysm sac.

The left portion of FIG. 56 shows this device at a first time, wherein wire 5602 is pulled and/or wire 5604 is pushed, causing the right side of the mesh to expand more than the left side of the mesh. The right portion of FIG. 56 shows this device at a second time, wherein wire 5602 is pushed and/or wire 5604 is pulled, causing the left side of the mesh to expand more than the right side of the mesh.

In an example, a device operator can control asymmetric expansion of the mesh by selectively pulling or pushing one or more wires which are connected to different locations on the mesh. In an example, a device operator can steer the mesh in a selected direction, at a selected angle, or with a selected orientation by selectively pulling or pushing one or more wires which are connected to different locations on the mesh. In an example, there can be two wires, one wire connected to each side (e.g. opposite sides) of the expandable mesh. In an example, there can be three or more wires connected to different (e.g. equidistant) locations around the circumference of the expandable mesh. Relevant variations discussed elsewhere in this disclosure or in priority-linked disclosures can also be applied to this example.

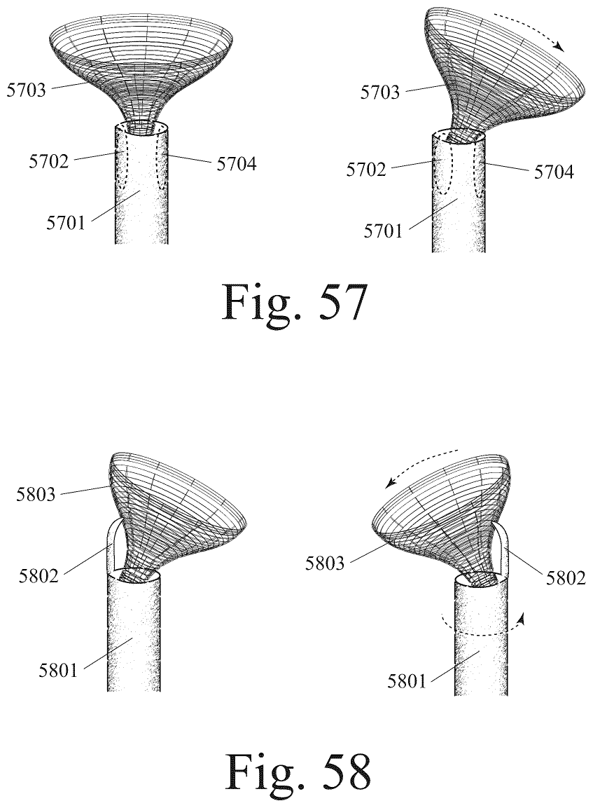

FIG. 57 shows two views, at two different times, of an intrasaccular aneurysm occlusion device comprising: an expandable mesh (e.g. mesh. lattice, braid, stent, and/or neck bridge) 5703 that is inserted and expanded within an aneurysm sac; a catheter 5701 through which the expandable mesh is delivered to the aneurysm sac; and a plurality of fluid-filled chambers 5702 and 5704 in the interior of the catheter, wherein selective filling of one or more of the fluid-filled chambers with a fluid causes the mesh to expand in an asymmetric manner and/or steers the mesh in a selected direction, at a selected angle, or with a selected orientation. The left portion of FIG. 57 shows this device at a first time, when neither of the fluid-filled chambers is filled and/or expanded. The right portion of FIG. 57 shows this device at a first second, when fluid-filled chamber 5702 has been s is filled and/or expanded. Relevant variations discussed elsewhere in this disclosure or in priority-linked disclosures can also be applied to this example.

FIG. 58 shows two views, at two different times, of an intrasaccular aneurysm occlusion device comprising: an expandable mesh (e.g. mesh, lattice, braid, stent, and/or neck bridge) 5803 that is inserted and expanded within an aneurysm sac; a catheter 5801 through which the expandable mesh is delivered to the aneurysm sac, wherein the distal end of the catheter further comprises a radially-inward protrusion 5802, and wherein rotation of the catheter causes the mesh to expand in an asymmetric manner and/or steers the mesh in a selected direction, at a selected angle, or with a selected orientation. The left portion of FIG. 58 shows this device at a first time when the catheter has a first rotational orientation. The right portion of FIG. 58 shows this device at a second time when the catheter has a second rotational orientation Relevant variations discussed elsewhere in this disclosure or in priority-linked disclosures can also be applied to this example.

I claim:

1. An intrasaccular aneurysm occlusion device comprising:
an expandable mesh that is configured to be inserted into an aneurysm sac;
embolic members or material that are inserted into the aneurysm sac; and
a catheter through which the expandable mesh and/or the embolic members or material are delivered to the aneurysm sac.

2. The device in claim 1 wherein the device further comprises a distal end portion of the catheter and a pivoting and/or rotating joint between the distal end portion and the rest of the catheter, and wherein the expandable mesh is steered in a selected direction and/or expanded in a radially-asymmetric manner by pivoting and/or rotating the joint.

3. The device in claim 1 wherein the device further comprises a first partially-circumferential section of the catheter and a second partially-circumferential section of the catheter, wherein the expandable mesh is steered in a selected direction and/or expanded in a radially-asymmetric manner by sliding the first partially-circumferential section relative to the second partially-circumferential section.

4. The device in claim 1 wherein the device further comprises a plurality of wires or cords which are attached to different locations on the expandable mesh, wherein the expandable mesh is steered in a selected direction and/or expanded in a radially-asymmetric manner by selectively pulling, pushing, or rotating a subset of one or more wires or cords in the plurality of wires or cords.

5. The device in claim 1 wherein the device further comprises a plurality of detachable constraints on the expandable mesh which constrain radial expansion of the expandable mesh, and wherein the expandable mesh is steered in a selected direction and/or expanded in a radially-asymmetric manner by selective detachment of a subset of one or more of the detachable constraints.

6. The device in claim 1 wherein the expandable mesh has two or more layers, wherein the expandable has gaps or chambers between mesh layers, and wherein the expandable mesh is steered in a selected direction and/or expanded in a radially-asymmetric manner by selectively filling a subset of one or more of the gaps or chambers with embolic members or material.

7. The device in claim 1 wherein there are a plurality of openings with valves in the expandable mesh, wherein selected valves can be opened or closed by a device operator, and wherein embolic members or material are inserted into the aneurysm sac through a subset of one or more selected openings whose valves have been opened by the device operator.

8. The device in claim 7 wherein the plurality of openings includes a plurality of non-central openings.

9. The device in claim 1 wherein the expandable mesh is a composite mesh which further comprises a proximal-to-distal series of connected expandable meshes.

10. The device in claim 1 wherein the expandable mesh further comprises a proximal-to-distal series of lobes or undulations created by radially-constraining a continuous tubular mesh at multiple longitudinal locations.

11. An intrasaccular aneurysm occlusion device comprising:
a flexible net or mesh that is inserted into an aneurysm sac;
an expandable band or ring that is inserted into the aneurysm sac, wherein the expandable band or ring spans a circumference of the flexible net or mesh;
embolic members or material which are inserted into the flexible net or mesh; and
a catheter through which the flexible net or mesh, the expandable band or ring, and/or the embolic members or material are delivered to the aneurysm sac.

12. The device in claim 11 wherein the expandable band or ring is outside the flexible net or mesh.

13. The device in claim 11 wherein the expandable band or ring is inside the flexible net or mesh.

14. The device in claim 11 wherein the expandable band or ring has a proximal-to-distal width which is between 5% and 20% of a proximal-to-distal distance from a neck of the aneurysm to the peak of the dome of the aneurysm.

15. The device in claim 11 wherein there is an opening in the flexible net or mesh through which embolic members or material are inserted into the flexible net or mesh.

16. An intrasaccular aneurysm occlusion device comprising:
an expandable mesh that is inserted into an aneurysm sac; wherein the expandable mesh is radially expanded within the aneurysm sac at a first time into a first configuration having a convex shape; wherein the expandable mesh is collapsed, compressed, and/or inverted within the aneurysm sac at a second time into a second configuration having a concave shape; wherein the expandable mesh further comprises a flexible annular section or band which spans a circumference of the expandable mesh; wherein the flexible annular section of band is more flexible and/or elastic than the rest of the expandable mesh; wherein a first portion of the expandable mesh is distal relative to the flexible annular section or band in the first configuration; wherein a second portion of the expandable mesh is proximal relative to the flexible annular section or band in the first configuration; wherein the first portion of the expandable mesh is collapsed, compressed, and/or inverted into the second portion of the expandable mesh when the expandable mesh is changed from the first configuration to the second configuration; wherein both the first portion of the expandable mesh and the second portion of the expandable mesh are proximal relative to the flexible annular section or band in the second configuration;
embolic members or material that are inserted into portions of the aneurysm sac which are distal relative to the expandable mesh; wherein pressure from accumulation of the embolic members or material collapses, compresses, and/or inverts the first portion of the expandable mesh into the second portion of the expandable mesh; and
one or more catheters which deliver the expandable mesh and/or the embolic members or material to the aneurysm sac.

17. The device in claim 16 wherein the flexible annular section or band comprises between 2% and 10% of the expandable mesh.

18. The device in claim 16 wherein the flexible annular section or band comprises between 5% and 20% of the expandable mesh.

19. The device in claim 16 wherein mesh components in the flexible annular section are more flexible, more elastic, thinner, smaller, and/or more-sparsely woven or braided than mesh components in the rest of the expandable mesh.

20. The device in claim 19 wherein the mesh components are woven or braided wires, tubes, or filaments.
